(19) United States
(12) Patent Application Publication
(10) Pub. No.: US 2026/0337177 A1
(43) Pub. Date: Oct. 29, 2026

(54) TEAD CORE INHIBITORS FOR CANCER THERAPEUTICS

(71) Applicants: University of Florida Research Foundation, Incorporated, Gainesville, FL (US); The Administrators of The Tulane Educational Fund, New Orleans, LA (US)

(72) Inventors: Chenglong Li, Gainesville, FL (US); Liya Pi, New Orleans, LA (US); Chunbao Sun, New Orleans, LA (US); Chen Zhou, Gainesville, FL (US)

(73) Assignees: University of Florida Research Foundation, Incorporated, Gainesville, FL (US); The Administrators of The Tulane Educational Fund, New Orleans, LA (US)

(21) Appl. No.: 19/476,258

(22) PCT Filed: Apr. 19, 2024
(86) PCT No.: PCT/US2024/025477

§ 371 Date: Oct. 17, 2025

Related U.S. Application Data
(60) Provisional application No. 63/460,846, filed on Apr. 20, 2023.

(51) Int. Cl. C07D 401/12; A61K 31/4045; A61K 31/44; A61K 31/4709; A61P 35/00; C07D 209/12; C07D 209/14; C07D 209/20

(52) U.S. Cl. CPC C07D 401/12; A61K 31/4045; A61K 31/44; A61K 31/4709; A61P 35/00; C07D 209/12; C07D 209/14; C07D 209/20

(57) ABSTRACT

The present disclosure provides compounds of the formulae herein (e.g., Formulae I and II), and salts, solvates, hydrates, polymorphs, co-crystals, tautomers, stereoisomers, isotopically labeled compounds, and prodrugs thereof, which are inhibitors of the YAP-TEAD transcriptional complex. The present disclosure also provides pharmaceutical compositions and kits comprising the compounds, and salts, solvates, hydrates, polymorphs, co-crystals, tautomers, stereoisomers, isotopically labeled compounds, and prodrugs thereof, and methods of treating or preventing diseases by administering to a subject in need thereof the compounds, and salts, solvates, hydrates, polymorphs, co-crystals, tautomers, stereoisomers, isotopically labeled compounds, and prodrugs thereof, or pharmaceutical compositions thereof.

### CROSS-REFERENCE TO RELATED APPLICATIONS

This application claims the benefit of and priority under 35 U.S.C. § 119(e) to U.S. Provisional Application No. 63/460,846, filed Apr. 20, 2023, titled TEAD CORE INHIBITORS FOR CANCER THERAPEUTICS, the contents of which are incorporated herewith by reference in their entirety.

### GOVERNMENT SUPPORT

The invention was made with government support under grant number R01 AA028035 awarded by the National Institutes of Health and grant number HT92425-23-1-0737 awarded by U.S. Army Medical Research Acquisition Activity. The government has certain rights in the invention.

### BACKGROUND

Abnormal activation of the YAP transcriptional signaling pathway and/or deactivation of Hippo signaling have been shown to drive the proliferation of various cancers, including hepatocellular carcinoma (HCC) and hepatoblastoma (HB) cases. Resistance and toxicity are serious drawbacks that have been observed upon use of the current first- and second-line treatment options for cancer, therefore warranting the investigation of alternative therapeutic approaches.

### SUMMARY OF THE INVENTION

In some aspects, provided herein are compounds of Formula I and II:

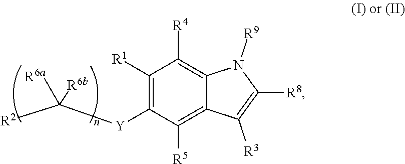

and salts, solvates, hydrates, polymorphs, co-crystals, tautomers, stereoisomers, isotopically labeled compounds, and prodrugs thereof, wherein R¹, R², R³, R⁴, R⁵, R^{6a}, R^{6b}, R⁸, R⁹, n, and Y are as provided herein.

In another aspect, provided herein is a compound of formula 7:

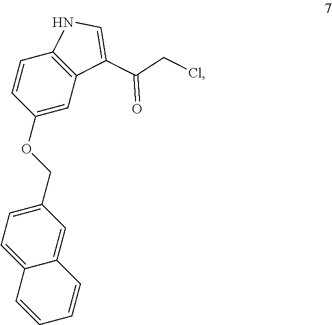 [SMILES: O=C(CCl)c1c[nH]c2ccc(OCc3ccc4ccccc4c3)cc12]

or a salt, solvate, hydrate, polymorph, co-crystal, tautomer, stereoisomer, isotopically labeled compound, or prodrug thereof.

In another aspect, provided herein is a compound of formula 12:

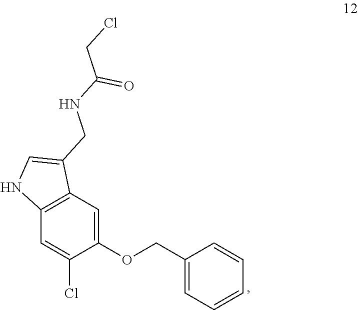 [SMILES: O=C(CCl)NCc1c[nH]c2cc(Cl)c(OCc3ccccc3)cc12]

or a salt, solvate, hydrate, polymorph, co-crystal, tautomer, stereoisomer, isotopically labeled compound, or prodrug thereof.

In another aspect, provided herein is a compound of formula 22:

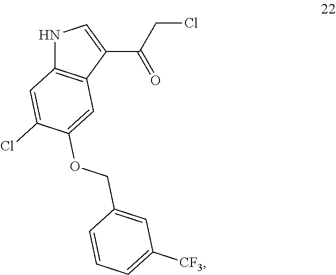 [SMILES: O=C(CCl)c1c[nH]c2cc(Cl)c(OCc3cccc(C(F)(F)F)c3)cc12]

or a salt, solvate, hydrate, polymorph, co-crystal, tautomer, stereoisomer, isotopically labeled compound, or prodrug thereof.

In another aspect, provided herein is a pharmaceutical composition comprising a compound provided herein, or a salt, solvate, hydrate, polymorph, co-crystal, tautomer, stereoisomer, isotopically labeled compound, or prodrug thereof; and optionally a pharmaceutically acceptable excipient.

In another aspect, provided herein is a kit comprising: a compound provided herein, or a salt, solvate, hydrate, polymorph, co-crystal, tautomer, stereoisomer, isotopically labeled compound, or prodrug thereof, or a pharmaceutical composition provided herein; and instructions for using the compound, or salt, solvate, hydrate, polymorph, co-crystal, tautomer, stereoisomer, isotopically labeled compound, or prodrug thereof, or pharmaceutical composition.

In another aspect, provided herein is a method of treating a disease in a subject in need thereof, the method comprising administering to the subject an effective amount of: a compound provided herein, or salt, solvate, hydrate, polymorph, co-crystal, tautomer, stereoisomer, isotopically labeled compound, or prodrug thereof; or a pharmaceutical composition provided herein.

In another aspect, provided herein is a method of preventing a disease in a subject in need thereof, the method comprising administering to the subject an effective amount of: a compound provided herein, or salt, solvate, hydrate, polymorph, co-crystal, tautomer, stereoisomer, isotopically labeled compound, or prodrug thereof; or a pharmaceutical composition provided herein.

In another aspect, provided herein is a method of inhibiting the activity of TEAD in a subject in need thereof, the method comprising administering to the subject an effective amount of: a compound provided herein, or salt, solvate, hydrate, polymorph, co-crystal, tautomer, stereoisomer, isotopically labeled compound, or prodrug thereof; or a pharmaceutical composition provided herein.

In another aspect, provided herein is a method of inhibiting the activity of TEAD in a cell, tissue, or biological sample the method comprising contacting the cell, tissue, or biological sample with an effective amount of: a compound provided herein, or salt, solvate, hydrate, polymorph, co-crystal, tautomer, stereoisomer, isotopically labeled compound, or prodrug thereof; or a pharmaceutical composition provided herein.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description, provide non-limiting examples of the invention.

FIGS. 1A-1D depict discovery of C-3 as moderate TEAD transcription inhibitor. (FIG. 1A) The flowchart of the docking-based virtual screening campaign. (FIG. 1B) Predicted binding mode of C-3 in TEAD2 lipid binding pocket. (FIG. 1C)C-3 dose dependently inhibited TEADs transcription activity in TEADs specific luciferase reporter assay. (FIG. 1D) Fluorescence polarization assay shows inhibition by C-3.

FIG. 2 shows the design strategy of first series of covalent TEAD inhibitors.

FIGS. 3A-3F show cellular activities of some first series of compounds. (FIGS. 3A-3B) The effect of compounds 1, 2, 3 and 5 on 8×GTIIC-luciferase reporter systems in Huh7 cells (FIGS. 3A and 3B). (FIG. 3C) compound 2 showed TEADs transcription inhibtion with IC₅₀890 nM. (FIG. 3D) Quantitative RT-PCR analysis demonstrated the downregulation of CTGF upon treatment with compound 2. (FIGS. 3E-3F) CTGF expression was significantly downregulated by compound 2 in a dose dependent manner in the Huh7 cell line.

FIGS. 4A-4G show cellular activity of compounds 22 and 12. (FIGS. 4A-4D) The effect of compounds 12 and 22 on TEAD-dependent reporter system. (FIG. 4E) CTGF gene expression was significantly downregulated by compound 22 in a dose dependent manner in both Huh7 and HepG2 cell lines. (FIGS. 4F-4G) CTGF expression was significantly downregulated by Compounds 12 and 22 in a dose dependent manner in the Huh7 cell line.

FIG. 5 shows proliferation inhibition of different cell lines by compound 22 in vitro. Error bars represent SD for three replicates.

FIGS. 6A-6D show in vivo antitumor activity of compound 22 in Huh7 cell-derived xenograft. Tumor volume (FIG. 6A), tumor weight (FIG. 6B), and body weight (FIG. 6C) of the mice after treatment of compound 22, n=2 mice per group. (FIG. 6D) The expression level of CTGF were detected by western blotting. GAPDH was used as a loading control. *p<0.05.

FIG. 7 shows compound 22 inhibits hepatoblastoma HepG2 survival in MTT assay.

FIG. 8 shows compound 22 promotes HepG2 cell apoptosis detected by calcein AM and ethidium homodimer-1 staining.

FIG. 9 shows compound 22 inhibits hepatocellular carcinoma Huh7 viability in MTT assay.

FIG. 10 shows compound 22 promotes Huh7 cell apoptosis detected by calcein AM and ethidium homodimer-1 staining.

FIG. 11 shows bioluminescence imaging of Huh7 xenograft mouse model treated with 22.

FIG. 12 shows images from a hepatoblastoma mouse model treated with either vehicle or 22.

FIG. 13 shows NCI-H226 cell (NF2-deficient Mesothelioma) viability after treatment with 23 or 24.

FIG. 14 shows NCI-H226 cell (NF2-deficient Mesothelioma) viability after treatment with NCV-1.

FIG. 15 shows structures of compounds from literature targeting the YAP/TEAD complex.

FIG. 16 shows crystal structure of YAP-TEAD1 complex with interface 1, 2 and 3 (PDB code 3KYS). Several pivotal TEAD inhibitors are categorized based on their interactions with specific binding sites.

FIGS. 17A-17B show covalent interaction between TEAD2-YBD and compound 22. (FIG. 17A) Mass spectrometry analysis revealed the covalent binding of compound 22 to the TEAD2-YBD protein. (FIG. 17B) Lack of mass change upon incubation of compound 22 with the TEAD2-YBD C380S mutant variant.

FIG. 18 shows compound 22 disrupts TEAD interaction with YAP in cells. Co-IP of endogenous YAP and TEAD in NCI-H226 cells after treatment with compound 22 at indicated doses. Cells were treated for 24 hr.

FIGS. 19A-19D shows inhibitory effects of compound 22 on colony formation of Huh7 and HepG2 cells. (FIG. 19A) Huh7 cells were treated with 5 uM compound 22. Digital image was taken, and all visible colonies were counted. (FIG. 19B) Data were normalized to untreated control. The error bars represent standard deviations from triplicates. The statistical significance was obtained with one-way analysis of variance (ANOVA) (**,P<0.01). (FIG. 19C) HepG2 cells were treated with 5 uM compound 22. Digital image was taken, and all visible colonies were counted. (FIG. 19D) Data were normalized to untreated control. The error bars represent standard deviations from triplicates. The statistical significance was obtained with one-way analysis of variance (ANOVA) (**,P<0.01).

FIG. 20 shows compound 22 induces G1-phase arrest in Huh7 and HepG2 cells. Huh7 and HepG2 cells were treated with DMSO and 5, 10 μM of compound 22 for 48 h. The cells were harvested, stained with PI, and then analyzed by flow cytometry

FIG. 21 shows compound 22 induced apoptosis of Huh7 and HepG2 cells. Huh7 and HepG2 cells were incubated with varying concentrations of compound 22 (0, 5, 10 μM). After 48 h of incubation, cells were collected and stained with Annexcin V/PI, followed by flow cytometric analysis.

FIGS. 22A-22D shows in vivo antitumor vivo antitumor efficacy of compound 22, Sorafenib, and a combination of 22 with Sorafenib in a Huh7 cell-derived xenograft. Tumor volume (FIG. 22A), tumor weight (FIG. 22B), and body weight (FIG. 22C) of the mice after treatment of compound 22, n=5 mice per group. (FIG. 22D) The expression levels of CTGF and CYR61 were detected by western blotting. GAPDH was used as a loading control. ****p<0.0001.

FIGS. 23A-23D show compound 22 inhibits HB tumor growth. (FIG. 23A) Decreased development of HB tumors after 10-week compound 22 (25 mg/kg body weight) treatment (n=15) compared to Vehicle controls (n=15). Values represent mean±SD from 30 mice. **p<0.01. (FIG. 23B) shows Ki67 staining for Proliferating cells in Vehicle or compound 22-treated livers. Scale bar: 150 uM. Quantification was performed from 15 mice per group. **p<0.01. (FIG. 23C) Real time q-PCR analysis detects lower levels of Ccnd1, Ctgf, Cyr61, Afp, and Gpc3 in compound 22-treated murine livers compared to Vehicle controls. ***p<0.001; Student t test. (FIG. 23D) The expression levels of Ctgf and Cyr61 proteins were detected by western blotting. Gapdh was used as a loading control.

FIG. 24A-24B show structures of compounds targeting the TEAD2 hydrophobic pocket (FIG. 24A) and their TEAD transcription inhibition activities (FIG. 24B).

FIGS. 25A-25C show proliferation inhibition of indicated cell lines HepG2 and Huh7 (FIG. 25A), Hs578T (FIG. 25B), and NCI-H226 and NCI-H2452 (FIG. 25C) by compound 22 in vitro.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modem Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl

group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”).In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

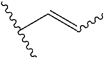

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has Ito 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and lor 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 xT electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 xT electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{bb})₂, —P(═O)(OR^{cc})₂, —OP(═O)(R^{bb})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{aa})₃⁺X⁻, —P(OR⁾₃⁺X⁻, —P(R^{cc})₄, —P(OR^{cc})₄, —OP(R^{cc})₂, —OP(R^{aa})₃⁺X⁻, —OP(OR^{cc})₂, —OP(OR⁾₃⁺X⁻, —OP(R^{cc})₄, —OP(OR^{cc})₄, —B(R^{bb})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{aa})₂, —C(═O)SR^{cc}, —C(═S)SR^{cc}, —P(═O)(R^{bb})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀ alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R)₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁0 alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form ═O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, SR^{aa}—N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{a}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{aa})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{aa}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di- aliphaticamino, mono- or di- heteroaliphaticamino, mono- or di- alkylamino, mono- or di- heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{aa})₂—CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{aa})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{aa})₂, —P(═O)(R^{bb})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{aa})₂, —P(R^{aa})₃⁺X⁻, —P(OR^{cc})₂, —P(OR⁾₃⁺X⁻, —P(═O)(R^{aa})₂, —P(═O)(OR⁾₂, and —P(═O)(N(R^{bb})₂)₂, wherein X⁻, R^{aa} R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{bb})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. Additional terms may be defined in other sections of this disclosure.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference.

Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting TEAD. In certain embodiments, a therapeutically effective amount is an amount sufficient for treating cancer. In certain embodiments, a therapeutically effective amount is an amount sufficient for inhibiting TEAD and treating cancer.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting TEAD. In certain embodiments, a prophylactically effective amount is an amount sufficient for preventing cancer. In certain embodiments, a prophylactically effective amount is an amount sufficient for inhibiting TEAD and preventing cancer.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein the term “inhibit” or “inhibition” in the context of enzymes, for example, in the context of TEAD, refers to a reduction in activity. In some embodiments, the term refers to a reduction of the level of activity, e.g., TEAD activity, to a level that is statistically significantly lower than an initial level, which may, for example, be a baseline level of activity. In some embodiments, the term refers to a reduction of the level of activity, e.g., TEAD activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development.

Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites.

Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

### Compounds

In one aspect, provided herein is a compound of Formula II:

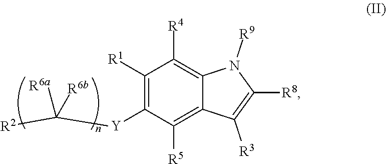

or a salt, solvate, hydrate, polymorph, co-crystal, tautomer, stereoisomer, isotopically labeled compound, or prodrug thereof, wherein:
Y is —NR^{A}—, —O—, —S—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is

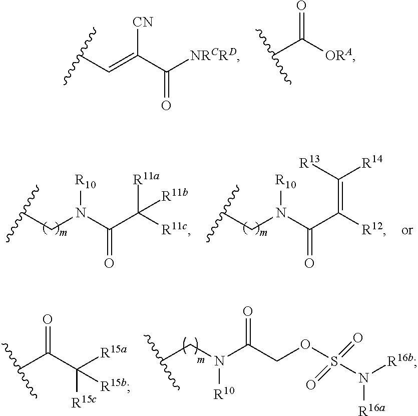

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{1a}, R^{1b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each of R^{16a} and R^{16b} is independently hydrogen, optionally substituted C₁₋₆ alkyl, or optionally substituted aryl;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

In another aspect, provided herein is a compound of Formula I:

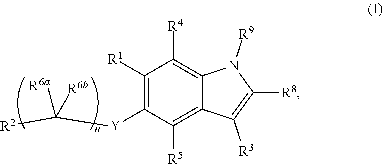

or a salt, solvate, hydrate, polymorph, co-crystal, tautomer, stereoisomer, isotopically labeled compound, or prodrug thereof, wherein:
Y is —NR^{A}—, —O—, —S—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is

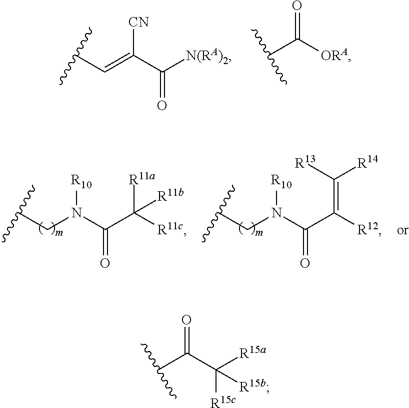

R⁴ is hydrogen or halogen;
R⁵ is hydrogen or halogen;
each of R^{6a} and R^{6b} is independently hydrogen, halogen, optionally substituted alkyl;
each of R^{7a} and R^{7b} is independently hydrogen, halogen, optionally substituted alkyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

In some embodiments, the compound is of Formula I-A:

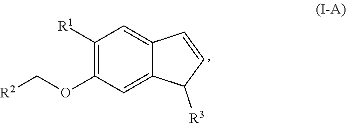

or a salt, solvate, hydrate, polymorph, co-crystal, tautomer, stereoisomer, isotopically labeled compound, or prodrug thereof, wherein:
R¹ is —Cl or —OMe;
R² is

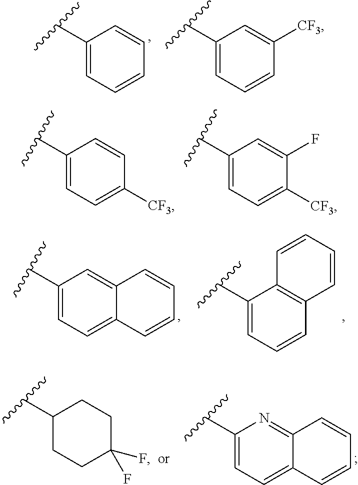

and
R³ is

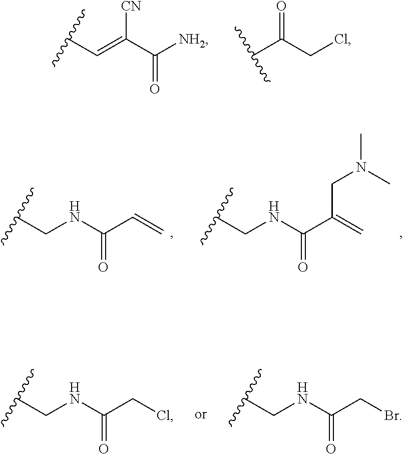

In some embodiments, the compound is of Formula II-B:

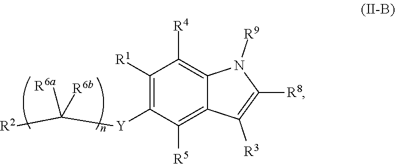

or a salt, solvate, hydrate, polymorph, co-crystal, tautomer, stereoisomer, isotopically labeled compound, or prodrug thereof, wherein:
Y is —NR^{A}-, —O—, or —CR^{7a}R^{7b}—;
R¹ is —F, —Br, —I, optionally substituted C₁₋₆ alkyl, optionally substituted carbocyclyl, —CN, —OR^{B}, —C(═O)OR^{A}, —Si(R^{A})₃, or —S(═O)₂R^{A}.
R² is optionally substituted C₁₋₆ alkyl, —OR^{A}, optionally substituted carbocyclyl, optionally substituted heterocyclyl, optionally substituted aryl, or optionally substituted heteroaryl;
R³ is

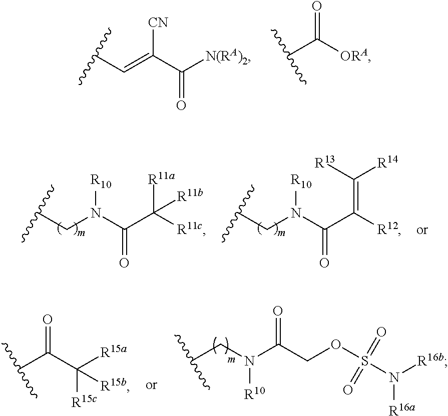

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each of R^{16a} and R^{16b} is independently hydrogen, optionally substituted C₁₋₆ alkyl, or optionally substituted aryl;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{B} is independently hydrogen, optionally substituted C₂-C₁₀ alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

In some embodiments, the compound is of Formula I-B:

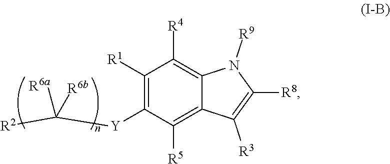

or a salt, solvate, hydrate, polymorph, co-crystal, tautomer, stereoisomer, isotopically labeled compound, or prodrug thereof,
wherein:
Y is —NR^{A}—, —O—, or —CR^{7a}R^{7b}—;
R¹ is —F, —Br, —I, optionally substituted C₁₋₆ alkyl, optionally substituted carbocyclyl, —CN, —OR^{B}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is

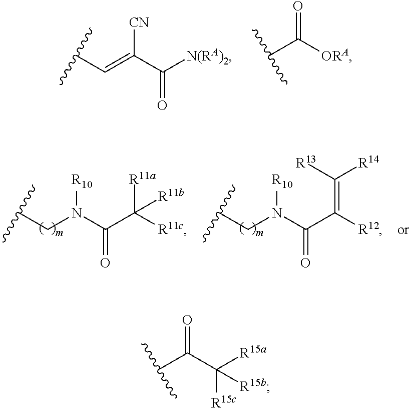

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{B} is independently hydrogen, optionally substituted C₂-C₁₀ alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

In some embodiments, the compound is of Formula II-C:

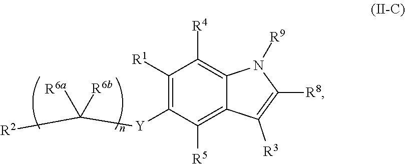

or a salt, solvate, hydrate, polymorph, co-crystal, tautomer, stereoisomer, isotopically labeled compound, or prodrug thereof,
wherein:
Y is —NR^{A}—, —O—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is

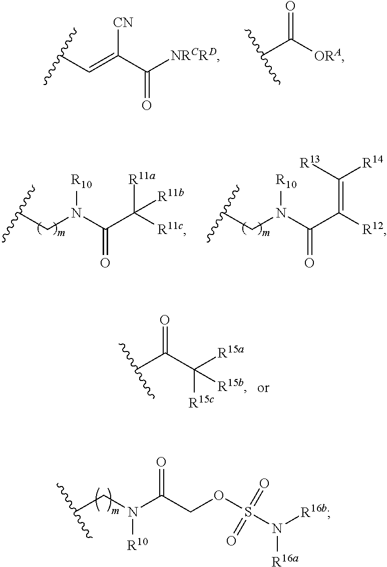

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen, —F, or —I;
R¹² is independently hydrogen, halogen, or —CN;
each of R¹³ and R¹⁴ is independently hydrogen, halogen, optionally substituted C₁₋₆ alkyl, optionally substituted C₁₋₆ heteroalkyl, or —CN;
provided that at least one of R¹², R¹³, and R¹⁴ is halogen, optionally substituted C₁₋₆ alkyl, optionally substituted C₁₋₆ heteroalkyl, or —CN;
each of R^{15a}, R^{15b}, and R^{15c}, is independently hydrogen, —F, —Br, or —I, provided that at least one of R^{15a}, R^{15b}, and R^{15c} is —F, —Br, or —I;
each of R^{16a} and R^{16b} is independently hydrogen, optionally substituted C₁₋₆ alkyl, or optionally substituted aryl;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{C} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring;
R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or R^{C} and R^{D} are joined together with the intervening atom to form an optionally substituted, monocyclic, heterocyclic or heteroaryl ring;
n is an integer from 0 to 1; and
m is an integer from 0 to 1.

In some embodiments, the compound is of Formula I-C:

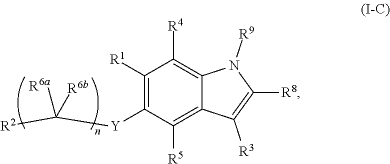

or a salt, solvate, hydrate, polymorph, co-crystal, tautomer, stereoisomer, isotopically labeled compound, or prodrug thereof,
wherein:
Y is —NR^{A}—, —O—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is

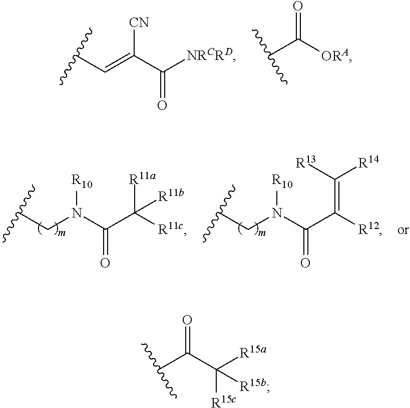

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen, —F, or —I;
R¹² is independently hydrogen, halogen, or —CN;
each of R¹³ and R¹⁴ is independently hydrogen, halogen, optionally substituted C₁₋₆ alkyl, optionally substituted C₁₋₆ heteroalkyl, or —CN;
provided that at least one of R¹², R¹³, and R¹⁴ is halogen, optionally substituted C₁₋₆ alkyl, optionally substituted C₁₋₆ heteroalkyl, or —CN;
each of R^{15a}, R^{15b}, and R^{15c}, is independently hydrogen, —F, —Br, or —I, provided that at least one of R^{15a}, R^{15b}, and R^{15c} is —F, —Br, or —I;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{C} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring;
R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or R^{C} and R^{D} are joined together with the intervening atom to form an optionally substituted, monocyclic, heterocyclic or heteroaryl ring;
n is an integer from 0 to 1; and
m is an integer from 0 to 1.

As generally defined herein, Y is —NR^{A}—, —O—, —S—, or —CR^{7a}R^{7b}—. In some embodiments, Y is —NH—, —NMe—, —O—, —S—, or —CH₂—. In some embodiments, Y is —NH—, —O—, —S—, or —CH₂—. In some embodiments, Y is —NR^{A}—, —O—, or —CR^{7a}R^{7b}—. In some embodiments, Y is —NR^{A}—, —O—, or —S—. In some embodiments, Y is —NR^{A}—, —S—, or —CR^{7a}R^{7b}—. In some embodiments, Y is —NMe—, —NH—, —S—, or —CH₂—. In some embodiments, Y is —O—. In some embodiments, Y is —S—. In some embodiments, Y is —NR^{A}—. In some embodiments, Y is —NH—. In some embodiments, Y is —NMe—. In some embodiments, Y is —CH₂—. In some embodiments, Y is —CHMe—. In some embodiments, Y is —CMe₂—.

As generally defined herein, R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A}. In some embodiments, R¹ is halogen, —CN, C₁₋₄ alkyl optionally substituted with one or more fluorine atoms, cyclopropyl, —C(═O)O(C₁₋₃ alkyl), —O(C₁₋₃ alkyl optionally substituted with one or more fluorine atoms), —Si(C₁₋₃ alkyl)₃, or —S(═O)₂(C₁₋₃ alkyl). In some embodiments, R¹ is —F, —Br, —I, optionally substituted C₁₋₆ alkyl, optionally substituted carbocyclyl, —CN, —OR^{B}, —C(═O)OR^{A}, —Si(R^{A})₃, or —S(═O)₂R^{A}. In some embodiments, R¹ is halogen or —O(C₁₋₃ alkyl optionally substituted with one or more fluorine atoms). In some embodiments, R¹ is —Cl or —OMe. In some embodiments, R¹ is —Cl. In some embodiments, R¹ is —OMe. In some embodiments, R¹ is —F, —Br, or —I. In some embodiments, R¹ is —O(C₂₋₃ alkyl optionally substituted with one or more fluorine atoms). In some embodiments, R¹ is —F, —Br, —I, —CN, C₁₋₄ alkyl optionally substituted with one or more fluorine atoms, cyclopropyl, —C(═O)O(C₁₋₃ alkyl), —O(C₂₋₃ alkyl optionally substituted with one or more fluorine atoms), —Si(C₁₋₃ alkyl)₃, or —S(═O)₂(C₁₋₃ alkyl). In some embodiments, R¹ is —CN, C₁₋₄ alkyl optionally substituted with one or more fluorine atoms, cyclopropyl, —C(═O)O(C₁₋₃ alkyl), —Si(C₁₋₃ alkyl)₃, or —S(═O)₂(C₁₋₃ alkyl).

As generally defined herein, R² is optionally substituted C₁₋₆ alkyl, —OR^{A}, optionally substituted carbocyclyl, optionally substituted heterocyclyl, optionally substituted aryl, or optionally substituted heteroaryl. In some embodiments, R² is C₁₋₆ alkyl optionally substituted with one or more fluorine atoms, —O(C₁₋₅ alkyl optionally substituted with one or more fluorine atoms), optionally substituted C₆₋₁₀ aryl, optionally substituted 5-10-membered heteroaryl, optionally substituted C₄₋₈ cycloalkyl, or optionally substituted 3-7-membered heterocyclyl. In some embodiments, R² is phenyl, wherein the phenyl is optionally substituted by one or more instances of halogen; C₁₋₄ alkyl optionally substituted with one or more fluorine atoms; C₃₋₆ cycloalkyl; —O(C₁₋₄ alkyl optionally substituted with one or more fluorine atoms); pentafluorosulfanyl; —CN; —Si(C₁₋₃ alkyl); —S(═O)₂(C₁₋₃ alkyl); phenyl optionally substituted by one or more —CF₃ groups; —CO₂(C₁₋₄ alkyl); or —S(C₁₋₄alkyl). In some embodiments, R² is 3-7-membered heterocyclyl optionally substituted by halogen, C₁₋₄ fluoroalkyl, or phenyl optionally substituted by one or more fluorine atoms or —CF₃ groups. In some embodiments, R² is optionally substituted phenyl or optionally substituted naphthyl. In some embodiments, R² is phenyl optionally substituted either with halogen or with C₁₋₄ alkyl optionally substituted with one or more halogen atoms. In some embodiments, R² is phenyl optionally substituted with —F, —CF₃, —CHF₂, —CH₂F, or —Me. In some embodiments, R² is naphthyl optionally substituted either with halogen or with C₁₋₄ alkyl optionally substituted with one or more halogen atoms. In some embodiments, R² is naphthyl optionally substituted with —F, —CF₃, —CHF₂, —CH₂F, or —Me. In some embodiments, R² is

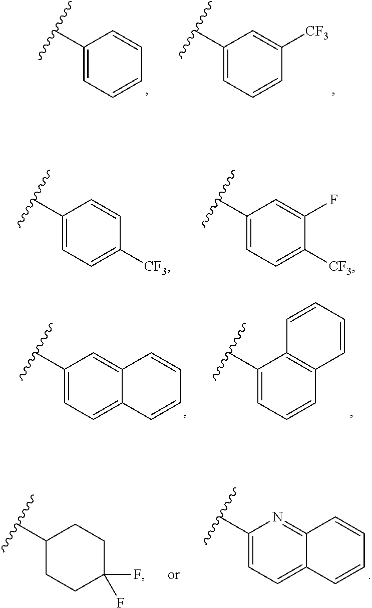

In some embodiments, R² is C₄₋₈ cycloalkyl optionally substituted by halogen or C₁₋₄ alkyl optionally substituted with one or more fluorine atoms. In some embodiments, R² is

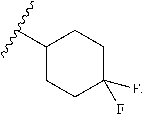 [SMILES: *C1CCC(F)(F)CC1]

In some embodiments, R² is

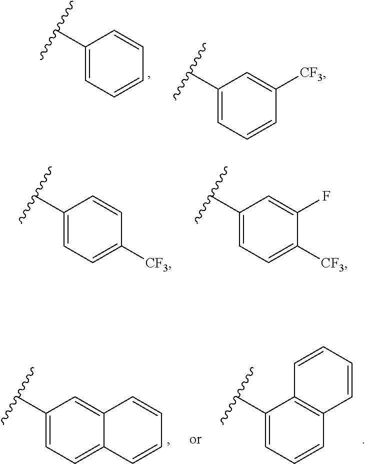 [SMILES: *c1ccccc1 ; *c1cccc(C(F)(F)F)c1 ; *c1ccc(C(F)(F)F)cc1 ; *c1ccc(C(F)(F)F)c(F)c1 ; *c1ccc2ccccc2c1 ; *c1cccc2ccccc12]

In some embodiments, R² is

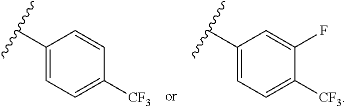 [SMILES: *c1ccc(C(F)(F)F)cc1 ; "or" ; *c1ccc(C(F)(F)F)c(F)c1]

As generally defined herein, R³ is

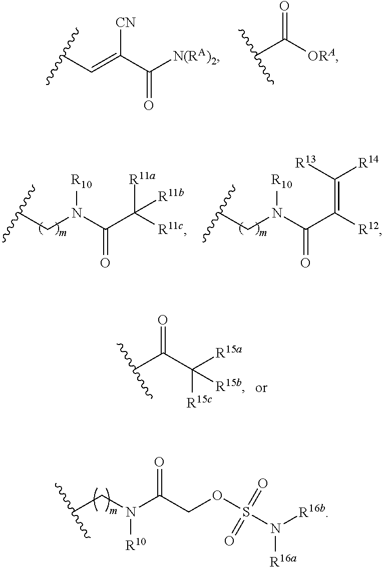

In some embodiments, R³ is

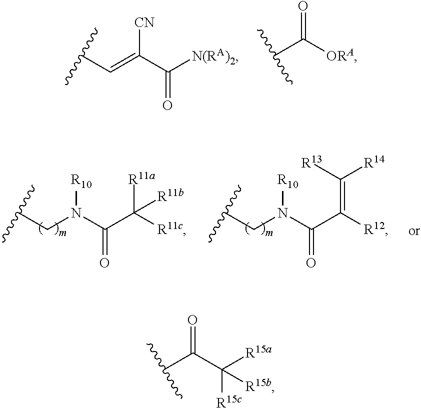

In some embodiments, R³ is

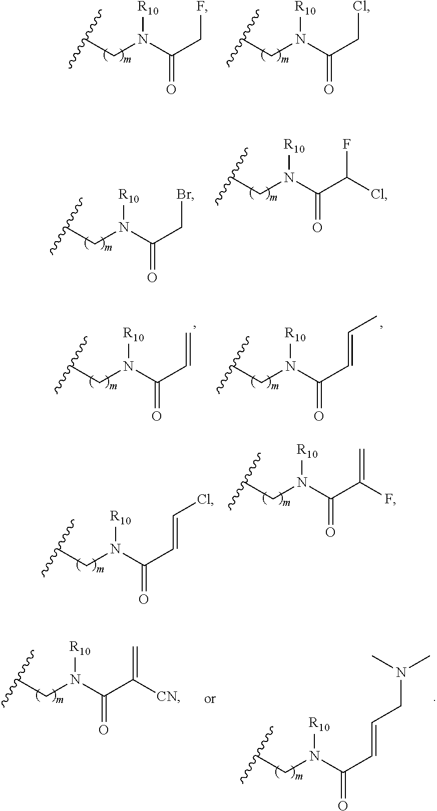

In some embodiments, R³ is

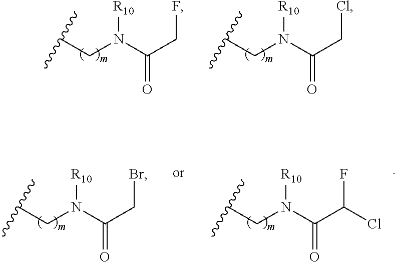

In some embodiments, R³ is

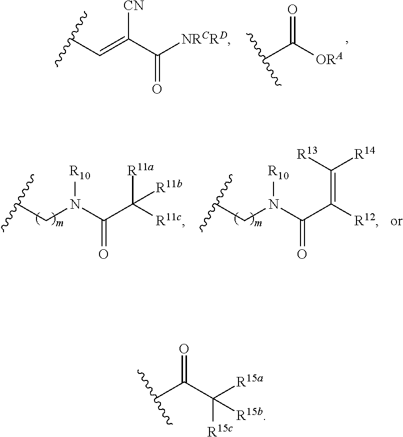

In some embodiments, R³ is

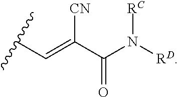

In some embodiments, R³ is

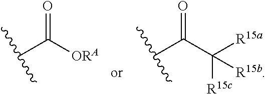

In some embodiments, R³ is

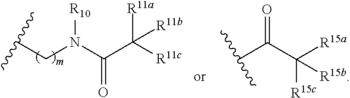

In some embodiments, R³ is

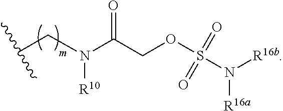

In some embodiments, R³ is

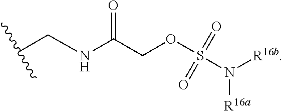

In some embodiments, R³ is

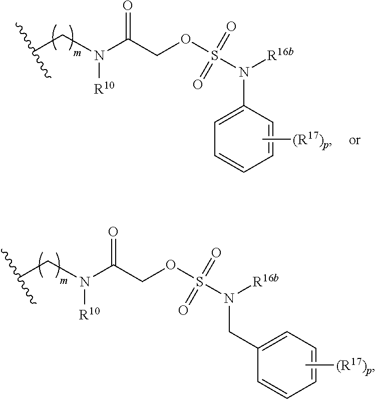

wherein p is an integer from 0 to 5, and each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, R³ is

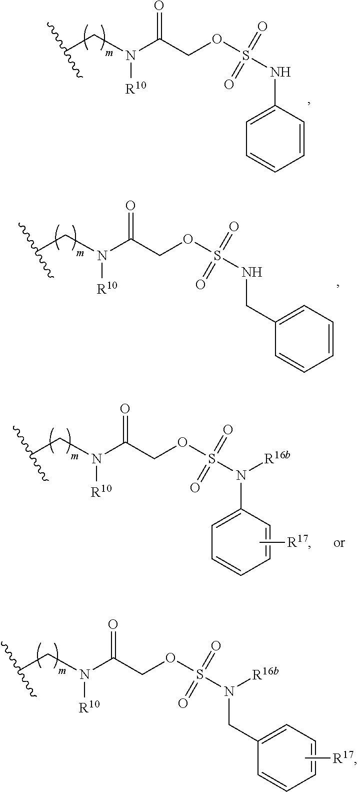

wherein R¹⁷ is independently halogen, —CN, optionally substituted C₁₋₂ alkyl, —OH, —O(optionally substituted C₁₋₂ alkyl), —C(═O)O(optionally substituted C₁₋₂ alkyl), or —C(═O)NH₂. In some embodiments, R³ is

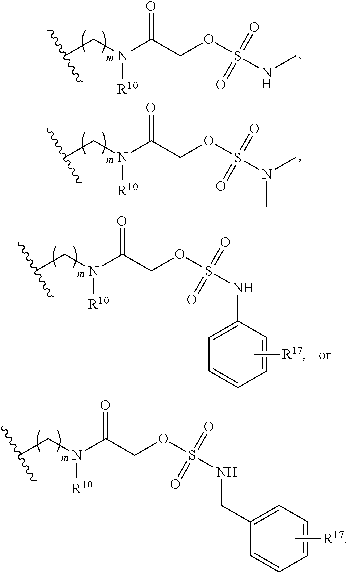

In some embodiments, R³ is

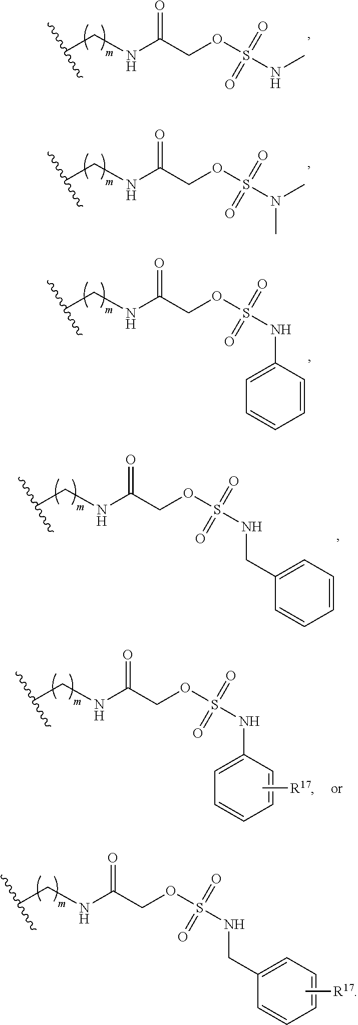

In some embodiments R³ is

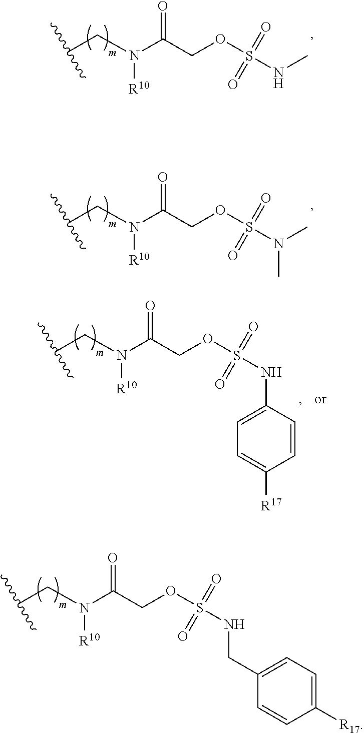

In some embodiments, R³ is

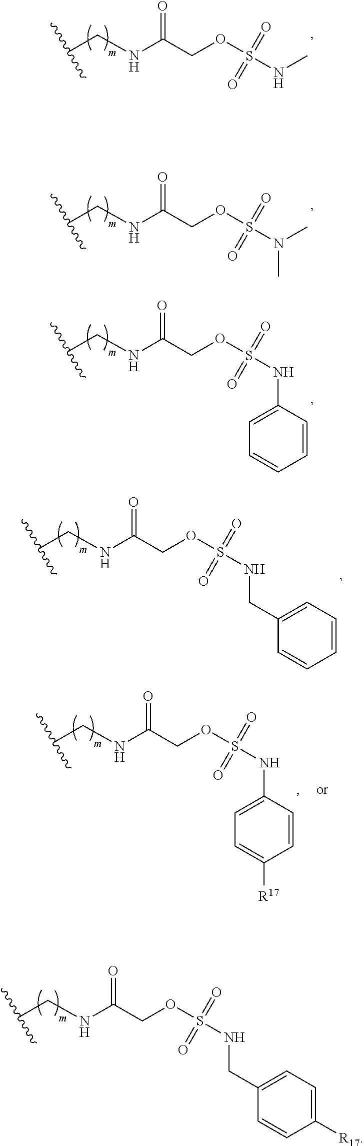

In some embodiments R³ is

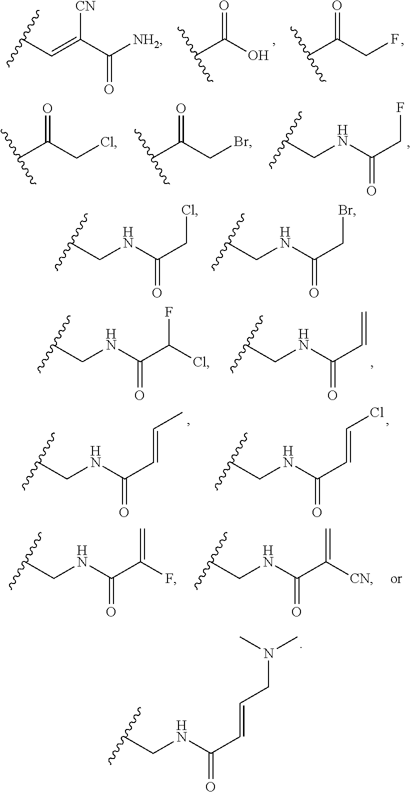

In some embodiments, R³ is

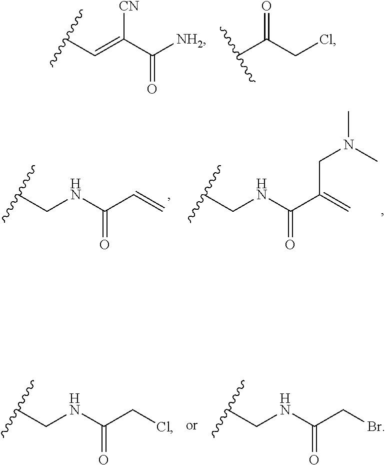

In some embodiments, R³ is

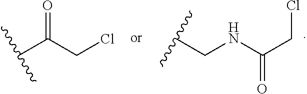 [SMILES: *C(=O)CCl ; "or" ; *CNC(=O)CCl]

In some embodiments, R³ is

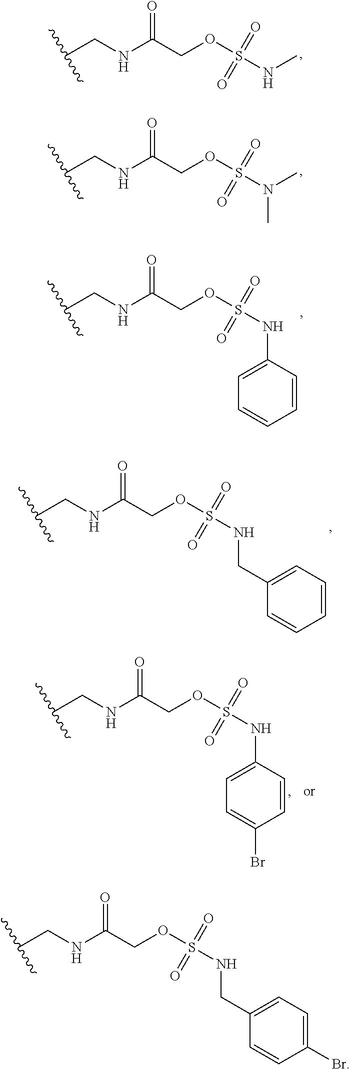 [SMILES: *CNC(=O)COS(=O)(=O)NC ; *CNC(=O)COS(=O)(=O)N(C)C ; *CNC(=O)COS(=O)(=O)Nc1ccccc1 ; *CNC(=O)COS(=O)(=O)NCc1ccccc1 ; *CNC(=O)COS(=O)(=O)Nc1ccc(Br)cc1 ; *CNC(=O)COS(=O)(=O)NCc1ccc(Br)cc1]

As generally defined herein, R⁴ is hydrogen or halogen. In some embodiments, R⁴ is hydrogen. In some embodiments, R⁴ is halogen. In some embodiments, R⁴ is hydrogen or —F.

In some embodiments, R⁴ is —F, —Cl, or —Br. In some embodiments, R⁴ is —F. In some embodiments, R⁴ is —Cl. In some embodiments, R⁴ is —Br. In some embodiments, R⁴ is —I.

As generally defined herein, R⁵ is hydrogen or halogen. In some embodiments, R⁵ is hydrogen. In some embodiments, R⁵ is halogen. In some embodiments, R⁵ is hydrogen or —F. In some embodiments, R⁵ is —F, —Cl, or —Br. In some embodiments, R⁵ is —F. In some embodiments, R⁵ is —Cl. In some embodiments, R⁵ is —Br. In some embodiments, R⁵ is —I.

As generally defined herein, each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{6a} and R^{6b} is independently hydrogen, halogen, or optionally substituted alkyl. In some embodiments, each of R^{6a} and R^{6b} is independently hydrogen or halogen. In some embodiments, each of R^{6a} and R^{6b} is independently hydrogen or —F. In some embodiments, each of R^{6a} and R^{6b} is independently hydrogen or optionally substituted C₁₋₃ alkyl. In some embodiments, R^{6a} and R^{6b} are each independently hydrogen, halogen, or C₁₋₃ alkyl. In some embodiments, R^{6a} and R^{6b} are each independently hydrogen, —F, —CF₃, —CF₂H, —CFH₂, —Me, -Et, -nPr, or -iPr. In some embodiments, at least one of R^{6a} and R^{6b} is hydrogen. In some embodiments, at least one of R^{6a} and R^{6b} is halogen. In some embodiments, at least one of R^{6a} and R^{6b} is —F. In some embodiments, at least one of R^{6a} and R^{6b} is C₁₋₃ alkyl. In some embodiments, one of R^{6a} and R^{6b} is hydrogen and the other is halogen or optionally substituted alkyl. In some embodiments, one of R^{6a} and R^{6b} is hydrogen and the other is halogen or optionally substituted C₁₋₃ alkyl. In some embodiments, one of R^{6a} and R^{6b} is hydrogen and the other is —F or C₁₋₃ alkyl. In some embodiments, R^{6a} and R^{6b} are each hydrogen. In some embodiments, R^{6a} and R^{6b} are each —F. In some embodiments, R^{6a} and R^{6b} are each —Me.

As generally defined herein, each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{7a} and R^{7b} is independently hydrogen, halogen, or optionally substituted alkyl. In some embodiments, each of R^{7a} and R^{7b} is independently hydrogen or halogen. In some embodiments, each of R^{7a} and R^{7b} is independently hydrogen or —F. In some embodiments, each of R^{7a} and R^{7b} is independently hydrogen or optionally substituted C₁₋₃ alkyl. In some embodiments, R^{7a} and R^{7b} are each independently hydrogen, halogen, or C₁₋₃ alkyl. In some embodiments, R^{7a} and R^{7b} are each independently hydrogen, —F, —CF₃, —CF₂H, —CFH₂, —Me, -Et, -nPr, or -iPr. In some embodiments, at least one of R^{7a} and R^{7b} is hydrogen. In some embodiments, at least one of R^{7a} and R^{7b} is halogen. In some embodiments, at least one of R^{7a} and R^{7b} is —F. In some embodiments, at least one of R^{7a} and R^{7b} is C₁₋₃ alkyl. In some embodiments, one of R^{7a} and R^{7b} is hydrogen and the other is halogen or optionally substituted alkyl. In some embodiments, one of R^{7a} and R^{7b} is hydrogen and the other is halogen or optionally substituted C₁₋₃ alkyl. In some embodiments, one of R^{7a} and R^{7b} is hydrogen and the other is —F or C₁₋₃ alkyl. In some embodiments, R^{7a} and R^{7b} are each hydrogen. In some embodiments, R^{7a} and R^{7b} are each —F. In some embodiments, R^{7a} and R^{7b} are each —Me.

As generally defined herein, R⁸ is hydrogen, optionally substituted C₁₋₆ alkyl, —CN, —OR^{A}, —C(═O)OR^{A}, or —C(═O)N(R^{A})₂. In some embodiments, R⁸ is hydrogen, C₁₋₃ alkyl, —CN, —OH, —O(C₁₋₃ alkyl), —C(═O)O(C₁₋₂ alkyl), or —CONH₂. In some embodiments, R⁸ is hydrogen or C₁₋₃ alkyl. In some embodiments, R⁸ is —OH or —O(C₁₋₃ alkyl). In some embodiments, R⁸ is —C(═O)OH, —C(═O)O(C₁₋₂ alkyl), —CON(C₁₋₂ alkyl)₂, —CONH(C₁₋₂ alkyl), or —CONH₂. In some embodiments, R⁸ is hydrogen. In some embodiments, R⁸ is optionally substituted C₁₋₆ alkyl. In some embodiments, R⁸ is optionally substituted C₁₋₃ alkyl. In some embodiments, R⁸ is C₁₋₃ alkyl. In some embodiments, R⁸ is —Me. In some embodiments, R⁸ is —CN. In some embodiments, R⁸ is —OR^{A}. In some embodiments, R⁸ is —O(C₁₋₃ alkyl). In some embodiments, R⁸ is —OH, —OMe, or —OEt. In some embodiments, R⁸ is —OH. In some embodiments, R⁸ is —CN. In some embodiments, R⁸ is —C(═O)OR^{A}. In some embodiments, R⁸ is —C(═O)O(C₁₋₂ alkyl) or —C(═O)OH. In some embodiments, R⁸ is —C(═O)N(R^{A})₂. In some embodiments, R⁸ is —CON(C₁₋₂ alkyl)₂, —CONH(C₁₋₂ alkyl), or —CONH₂.

As generally defined herein, R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group. In some embodiments, R⁹ is optionally substituted C₁₋₆ alkyl, optionally substituted C₂₋₆ alkenyl, or optionally substituted C₂₋₆ alkynyl. In some embodiments, R⁹ is optionally substituted C₁₋₆ heteroalkyl, optionally substituted C₂₋₆ heteroalkenyl, or optionally substituted C₂₋₆ heteroalkynyl. In some embodiments, R⁹ is optionally substituted C₃₋₇ carbocyclyl or optionally substituted 3- to 7-membered heterocyclyl. In some embodiments, R⁹ is optionally substituted C₃₋₇ carbocyclyl or optionally substituted C₆₋₁₀ aryl. In some embodiments, R⁹ is optionally substituted 3- to 7-membered heterocyclyl or optionally substituted 5- to 10-membered heteroaryl. In some embodiments, R⁹ is optionally substituted C₆₋₁₀ aryl or optionally substituted 5- to 10-membered heteroaryl. In some embodiments, R⁹ is hydrogen, optionally substituted alkyl, or a nitrogen protecting group. In some embodiments, R⁹ is hydrogen, optionally substituted C₁₋₆ alkyl, or a nitrogen protecting group. In some embodiments, R⁹ is a nitrogen protecting group. In some embodiments, R⁹ is hydrogen.

As generally defined herein, R¹⁰ is hydrogen or optionally substituted C₁₋₆ alkyl. In some embodiments, R¹⁰ is hydrogen or optionally substituted C₁₋₃ alkyl. In some embodiments, R¹⁰ is hydrogen, —Me, -Et, -nPr, or -iPr. In some embodiments, R¹⁰ is hydrogen. In some embodiments, R¹⁰ is optionally substituted C₁₋₆ alkyl. In some embodiments, R¹⁰ is optionally substituted C₁₋₃ alkyl. In some embodiments, R¹⁰ is —Me, -Et, -nPr, or -iPr.

As generally defined herein, each of R^{11a}, R^{11b}, and R^{11c} is independently hydrogen or halogen. In some embodiments, each of R^{11a}, R^{11b}, and R^{11c} is independently hydrogen, —F, or —I. In some embodiments, each of R^{11a}, R^{11b}, and R^{11c} is independently hydrogen or —F. In some embodiments, each of R^{11a}, R^{11b}, and R^{11c} is independently hydrogen or —Cl. In some embodiments, each of R^{11a}, R^{11b}, and R^{11c} is independently hydrogen or —Br. In some embodiments, each of R^{11a}, R^{11b}, and R^{11c} is independently hydrogen or —I. In some embodiments, at least one of R^{11a}, R^{11b}, and R^{11c} is hydrogen. In some embodiments, at least one of R^{11a}, R^{11b}, and R^{11c} is halogen. In some embodiments, at least one of R^{11a}, R^{11b}, and R^{11c} is —F, —Cl, or —Br. In some embodiments, at least two of R^{11a}, R^{11b}, and Rile are hydrogen. In some embodiments, at least two of R^{11a}, R^{11b}, and R^{11c} are halogen. In some embodiments, at least two of R^{11a}, R^{11b}, and Rile are —F, —Cl, or —Br.

As generally defined herein, R¹² is hydrogen, halogen, optionally substituted C₁₋₆ alkyl, optionally substituted C₁₋₆ heteroalkyl, or —CN. In some embodiments, R¹² is hydrogen, halogen, or —CN. In some embodiments, R¹² is hydrogen, optionally substituted C₁₋₆ alkyl, or optionally substituted C₁₋₆ heteroalkyl. In some embodiments, R¹² is hydrogen, C₁₋₃ alkyl, or C₁₋₃ heteroalkyl. In some embodiments, R¹² is hydrogen or C₁₋₃ alkyl. In some embodiments, R¹² is hydrogen. In some embodiments, R¹² is optionally substituted C₁₋₆ alkyl. In some embodiments, R¹² is optionally substituted C₁₋₃ alkyl. In some embodiments, R¹² is optionally substituted C₁₋₆ heteroalkyl. In some embodiments, R¹² is optionally substituted C₁₋₃ heteroalkyl. In some embodiments, R¹² is optionally substituted C₁₋₃ heteroalkyl comprising at least one nitrogen atom. In some embodiments, R¹² is —CH₂NMe₂.

As generally defined herein, R¹³ is hydrogen, halogen, optionally substituted C₁₋₆ alkyl, optionally substituted C₁₋₆ heteroalkyl, or —CN. In some embodiments, R¹³ is hydrogen, halogen, or —CN. In some embodiments, R¹³ is hydrogen, optionally substituted C₁₋₆ alkyl, or optionally substituted C₁₋₆ heteroalkyl. In some embodiments, R¹³ is hydrogen, C₁₋₃ alkyl, or C₁₋₃ heteroalkyl. In some embodiments, R¹³ is hydrogen or C₁₋₃ alkyl. In some embodiments, R¹³ is hydrogen. In some embodiments, R¹³ is optionally substituted C₁₋₆ alkyl. In some embodiments, R¹³ is optionally substituted C₁₋₃ alkyl. In some embodiments, R¹³ is optionally substituted C₁₋₆ heteroalkyl. In some embodiments, R¹³ is optionally substituted C₁₋₃ heteroalkyl.

As generally defined herein, R¹⁴ is hydrogen, halogen, optionally substituted C₁₋₆ alkyl, optionally substituted C₁₋₆ heteroalkyl, or —CN. In some embodiments, R¹⁴ is hydrogen, halogen, or —CN. In some embodiments, R¹⁴ is hydrogen, optionally substituted C₁₋₆ alkyl, or optionally substituted C₁₋₆ heteroalkyl. In some embodiments, R¹⁴ is hydrogen, C₁₋₃ alkyl, or C₁₋₃ heteroalkyl. In some embodiments, R¹⁴ is hydrogen or C₁₋₃ alkyl. In some embodiments, R¹⁴ is hydrogen. In some embodiments, R¹⁴ is optionally substituted C₁₋₆ alkyl. In some embodiments, R¹⁴ is optionally substituted C₁₋₃ alkyl. In some embodiments, R¹⁴ is optionally substituted C₁₋₆ heteroalkyl. In some embodiments, R¹⁴ is optionally substituted C₁₋₃ heteroalkyl.

In some embodiments, at least one of R¹², R¹³, and R¹⁴ is hydrogen. In some embodiments, at least one of R¹², R¹³, and R¹⁴ is halogen, optionally substituted C₁₋₆ alkyl, optionally substituted C₁₋₆ heteroalkyl, or —CN. In some embodiments, at least one of R¹² and R¹³ is hydrogen. In some embodiments, at least one of R¹² and R¹⁴ is hydrogen. In some embodiments, at least one of R¹³ and R¹⁴ is hydrogen. In some embodiments, R¹² and R¹³ are hydrogen. In some embodiments, R¹² and R¹⁴ are hydrogen. In some embodiments, R¹³ and R¹⁴ are hydrogen. In some embodiments, R¹², R¹³, and R¹⁴ are hydrogen.

As generally defined herein, each of R^{5a}, R^{15b}, and R^{15c}, is independently hydrogen or halogen, provided that at least one of R^{15a}, R^{15b}, and R¹⁵, is halogen. In some embodiments, each of R^{15a}, R^{15b}, and R^{15c}, is independently hydrogen, —F, —Br, or —I, provided that at least one of R^{15a}, R^{15b}, and R^{15c} is —F, —Br, or —I. In some embodiments, one of R^{15a}, R^{15b}, and R^{15c} is halogen. In some embodiments, one of R^{15a}, R^{15b}, and R^{15c} is hydrogen. In some embodiments, at least one of R^{15a}, R^{15b}, and R^{15c}, is —F. In some embodiments, at least one of R^{15a}, R^{15b}, and R¹⁵, is —Cl. In some embodiments, at least one of R^{15a}, R^{15b}, and R^{15c} is —Br. In some embodiments, two of R^{15a}, R^{15b}, and R^{15c} are hydrogen.

As generally defined herein, each of R^{16a} and R^{16b} is independently hydrogen, optionally substituted C₁₋₆ alkyl, or optionally substituted aryl. In some embodiments, each of R^{16a} and R^{16b} is independently hydrogen, optionally substituted C₁₋₆ alkyl, or optionally substituted phenyl. In some embodiments, each of R^{16a} and R^{16b} is independently hydrogen, optionally substituted C₁₋₃ alkyl, or optionally substituted phenyl. In some embodiments, each of R^{16a} and R^{16b} is independently hydrogen, optionally substituted C₁₋₃ alkyl, or

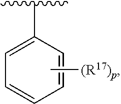

wherein p is an integer from 0 to 5, and each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, each of R^{16a} and R^{16b} is independently hydrogen, methyl, ethyl, n-propyl, i-propyl,

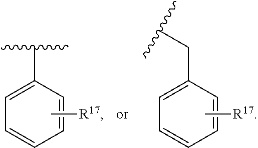

In some embodiments, at least one of R^{16a} and R^{16b} is hydrogen or methyl. In some embodiments, at least one of R^{16a} and R^{16b} is hydrogen. In some embodiments, at least one of R^{16a} and R^{16b} is methyl.

In some embodiments, R^{16a} is hydrogen, optionally substituted C₁₋₆ alkyl, or optionally substituted aryl. In some embodiments, R^{16a} is hydrogen, optionally substituted C₁₋₃ alkyl, or optionally substituted phenyl. In some embodiments, R^{16a} is hydrogen. In some embodiments, R^{16a} is optionally substituted C₁₋₆ alkyl. In some embodiments, R^{16a} is optionally substituted C₁₋₃ alkyl. In some embodiments, R^{16a} is C₁₋₃ alkyl optionally substituted with

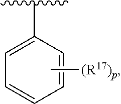

wherein p is an integer from 0 to 5, and each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂.

In some embodiments, R^{16a} is C₁₋₃ alkyl optionally substituted with

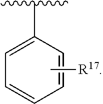

In some embodiments, R^{16a} is C₁₋₃ alkyl optionally substituted with

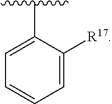

In some embodiments, R^{16a} is C₁₋₃ alkyl optionally substituted with

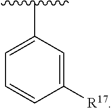

In some embodiments, R^{16a} is C₁₋₃ alkyl optionally substituted with

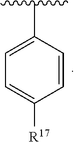

In some embodiments, R^{16a} is C₁₋₃ alkyl optionally substituted with

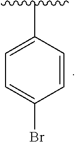 [SMILES: *c1ccc(Br)cc1]

In some embodiments, R^{16a} is optionally substituted aryl. In some embodiments, R^{16a} is optionally substituted phenyl. In some embodiments, R^{16a} is

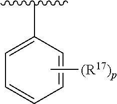

wherein p is an integer from 0 to 5, and each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, R^{16a} is

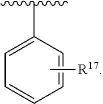

In some embodiments, R^{16a} is

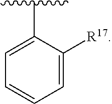

In some embodiments, R^{16a} is

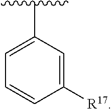

In some embodiments, R^{16a} is

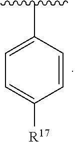

In some embodiments, R^{16a} is

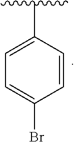 [SMILES: *c1ccc(Br)cc1]

In some embodiments, R^{16b} is hydrogen, optionally substituted C₁₋₆ alkyl, or optionally substituted aryl. In some embodiments, R^{16b} is hydrogen, optionally substituted C₁₋₃ alkyl, or optionally substituted phenyl. In some embodiments, R^{16b} is hydrogen. In some embodiments, R^{16b} is optionally substituted C₁₋₆ alkyl. In some embodiments, R^{16b} is optionally substituted C₁₋₃ alkyl. In some embodiments, R^{16b} is C₁₋₃ alkyl optionally substituted with

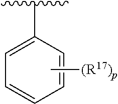

wherein p is an integer from 0 to 5, and each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, R^{16b} is C₁₋₃ alkyl optionally substituted with

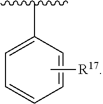

In some embodiments, R^{16b} is C₁₋₃ alkyl optionally substituted with

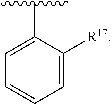

In some embodiments, R^{16b} is C₁₋₃ alkyl optionally substituted with

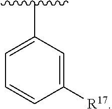

In some embodiments, R^{16b} is C₁₋₃ alkyl optionally substituted with

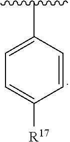

In some embodiments, R^{16b} is C₁₋₃ alkyl optionally substituted with

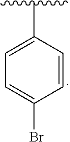 [SMILES: *c1ccc(Br)cc1]

In some embodiments, R^{16b} is optionally substituted aryl. In some embodiments, R^{16b} is optionally substituted phenyl. In some embodiments, R^{16b} is

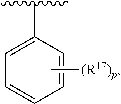

wherein p is an integer from 0 to 5, and each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, R^{16b} is

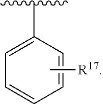

In some embodiments, R^{16b} is

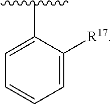

In some embodiments, R^{16b} is

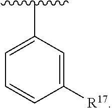

In some embodiments, R^{16b} is

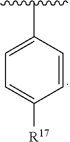

In some embodiments, R^{16b} is

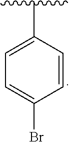 [SMILES: *c1ccc(Br)cc1]

As generally defined herein, each instance of R¹⁷ is independently halogen, —CN, optionally substituted C₁₋₂ alkyl, —OH, —O(optionally substituted C₁₋₂ alkyl), —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, each instance of R¹⁷ is independently halogen, —CN, optionally substituted C₁₋₂ alkyl, —OH, —O(optionally substituted C₁₋₂ alkyl), —C(═O)O(optionally substituted C₁₋₂ alkyl), or —C(═O)NH₂. In some embodiments, R¹⁷ is halogen. In some embodiments, R¹⁷ is —F, —Cl, or —Br. In some embodiments, R¹⁷ is —F. In some embodiments, R¹⁷ is —Cl. In some embodiments, R¹⁷ is —Br. In some embodiments, R¹⁷ is —CN. In some embodiments, R¹⁷ is —OH or —O(optionally substituted C₁₋₂ alkyl). In some embodiments, R¹⁷ is —OH, —OMe, or —OEt. In some embodiments, R¹⁷ is —OH, —OMe, or —OEt. In some embodiments, R¹⁷ is —C(═O)OH, —C(═O)O(optionally substituted C₁₋₂ alkyl), —C(═O)NH₂, —C(═O)NH(optionally substituted C₁₋₂ alkyl), or —C(═O)N(optionally substituted C₁₋₂ alkyl)₂. In some embodiments, R¹⁷ is —C(═O)OH or —C(═O)O(optionally substituted C₁₋₂ alkyl). In some embodiments, R¹⁷ is —C(═O)OH, —C(═O)OMe, or —C(═O)OEt. In some embodiments, R¹⁷ is —C(═O)NH₂, —C(═O)NH(optionally substituted C₁₋₂ alkyl), or —C(═O)N(optionally substituted C₁₋₂ alkyl)₂. In some embodiments, R¹⁷ is —C(═O)NH₂, —C(═O)NHMe, —C(═O)NHEt, —C(═O)N(Me)₂, —C(═O)N(Et)₂, or —C(═O)N(Me)Et.

As generally defined herein, each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring. In some embodiments, R^{A} is hydrogen. In some embodiments, R^{A} is optionally substituted C₁₋₆ alkyl, optionally substituted C₂₋₆ alkenyl, optionally substituted C₂₋₆ alkynyl. In some embodiments, R^{A} is optionally substituted C₁₋₆ heteroalkyl, optionally substituted C₂₋₆ heteroalkenyl, optionally substituted C₂₋₆ heteroalkynyl. In some embodiments, R^{A} is optionally substituted C₃₇ carbocyclyl or optionally substituted 3- to 7-membered heterocyclyl. In some embodiments, R^{A} is optionally substituted C₆₋₁₀ aryl or optionally substituted 5- to 10-membered heteroaryl. In some embodiments, R^{A} is optionally substituted phenyl. In some embodiments, R^{A} is optionally substituted 5- or 6-membered heteroaryl. In some embodiments, R^{A} is a nitrogen protecting group. In some embodiments, R^{A} is an oxygen protecting group. In some embodiments, R^{A} is a sulfur protecting group. In some embodiments, two instances of R^{A} attached to the same intervening atom are joined together with the intervening atom to form an optionally substituted, monocyclic 3- to 7-membered heterocyclic ring. In some embodiments, two instances of R^{A} attached to the same intervening atom are joined together with the intervening atom to form an optionally substituted, monocyclic, 5- or 6-membered heteroaryl ring.

As generally defined herein, R^{B} is independently hydrogen, optionally substituted C₂-C₁₀ alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring. In some embodiments, R^{B} is hydrogen. In some embodiments, R^{B} is optionally substituted C₂₋₆ alkyl, optionally substituted C₂₋₆ alkenyl, optionally substituted C₂₋₆ alkynyl. In some embodiments, R^{B} is optionally substituted C₁₋₆ heteroalkyl, optionally substituted C₂₋₆ heteroalkenyl, optionally substituted C₂₋₆ heteroalkynyl. In some embodiments, R^{B} is optionally substituted C3a carbocyclyl or optionally substituted 3- to 7-membered heterocyclyl. In some embodiments, R^{B} is optionally substituted C₆₋₁₀ aryl or optionally substituted 5- to 10-membered heteroaryl. In some embodiments, R^{B} is optionally substituted phenyl. In some embodiments, R^{B} is optionally substituted 5- or 6-membered heteroaryl. In some embodiments, R^{B} is a nitrogen protecting group. In some embodiments, R^{B} is an oxygen protecting group. In some embodiments, R^{B} is a sulfur protecting group. In some embodiments, two instances of R^{B} attached to the same intervening atom are joined together with the intervening atom to form an optionally substituted, monocyclic 3- to 7-membered heterocyclic ring. In some embodiments, two instances of R^{B} attached to the same intervening atom are joined together with the intervening atom to form an optionally substituted, monocyclic, 5- or 6-membered heteroaryl ring.

As generally defined herein, R^{C} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring. In some embodiments, R^{C} is optionally substituted C₁₋₆ alkyl, optionally substituted C₂₋₆ alkenyl, optionally substituted C₂₋₆ alkynyl. In some embodiments, R^{C} is optionally substituted C₁₋₆ heteroalkyl, optionally substituted C₂₋₆ heteroalkenyl, optionally substituted C₂₋₆ heteroalkynyl. In some embodiments, R^{C} is optionally substituted C₃₋₇ carbocyclyl or optionally substituted 3- to 7-membered heterocyclyl. In some embodiments, R^{C} is optionally substituted C₆₋₁₀ aryl or optionally substituted 5- to 10-membered heteroaryl. In some embodiments, R^{C} is optionally substituted phenyl. In some embodiments, R^{C} is optionally substituted 5- or 6-membered heteroaryl. In some embodiments, R^{C} is a nitrogen protecting group.

As generally defined herein, R^{D} is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring. In some embodiments, R^{D} is hydrogen. In some embodiments, R^{D} is optionally substituted C₁₋₆ alkyl, optionally substituted C₂₋₆ alkenyl, optionally substituted C₂₋₆ alkynyl. In some embodiments, R^{D} is optionally substituted C₁₋₆ heteroalkyl, optionally substituted C₂₋₆ heteroalkenyl, optionally substituted C₂₋₆ heteroalkynyl. In some embodiments, R^{D} is optionally substituted C₃₋₇ carbocyclyl or optionally substituted 3- to 7-membered heterocyclyl. In some embodiments, R^{D} is optionally substituted C₆₋₁₀ aryl or optionally substituted 5- to 10-membered heteroaryl. In some embodiments, R^{D} is optionally substituted phenyl. In some embodiments, R^{D} is optionally substituted 5- or 6-membered heteroaryl. In some embodiments, R^{D} is a nitrogen protecting group.

In some embodiments, R^{C} and R^{D} are joined together with the intervening atom to form an optionally substituted, monocyclic 3- to 7-membered heterocyclic ring. In some embodiments, R^{C} and R^{D} are joined together with the intervening atom to form an optionally substituted, monocyclic, 5- or 6-membered heteroaryl ring.

As generally defined herein, n is an integer from 0 to 1. In some embodiments, n is 0. In some embodiments, n is 1.

As generally defined herein, m is an integer from 0 to 1. In some embodiments, m is 0. In some embodiments, m is 1.

As generally defined herein, p is an integer from 0 to 5. In some embodiments, p is an integer from 0 to 4. In some embodiments, p is an integer from 1 to 5. In some embodiments, p is an integer from 0 to 3. In some embodiments, p is an integer from 1 to 4. In some embodiments, p is an integer from 2 to 5. In some embodiments, p is an integer from 0 to 2. In some embodiments, p is an integer from 1 to 3. In some embodiments, p is an integer from 2 to 4. In some embodiments, p is an integer from 3 to 5. In some embodiments, p is an integer from 0 to 1. In some embodiments, p is an integer from 1 to 2. In some embodiments, p is an integer from 2 to 3. In some embodiments, p is an integer from 3 to 4. In some embodiments, p is an integer from 4 to 5. In some embodiments, p is 0. In some embodiments, p is 1. In some embodiments, p is 2. In some embodiments, p is 3. In some embodiments, p is 4. In some embodiments, p is 5.

In some embodiments, the compound of Formula I, or a salt, solvate, hydrate, polymorph, co-crystal, tautomer, stereoisomer, isotopically labeled compound, or prodrug thereof, wherein the compound is any one of the formulae shown in Table 1:

TABLE 1

| | |
|---|---|
| 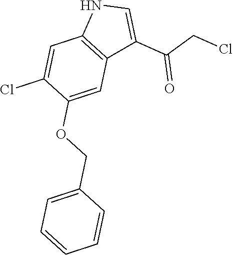 [SMILES: O=C(CCl)c1c[nH]c2cc(Cl)c(OCc3ccccc3)cc12] | 1 |
| 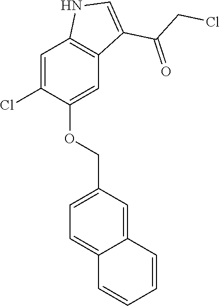 [SMILES: O=C(CCl)c1c[nH]c2cc(Cl)c(OCc3ccc4ccccc4c3)cc12] | 2 |
| 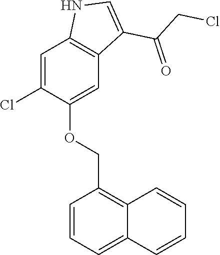 [SMILES: O=C(CCl)c1c[nH]c2cc(Cl)c(OCc3cccc4ccccc34)cc12] | 3 |
| 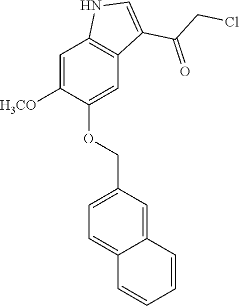 [SMILES: COc1cc2[nH]cc(C(=O)CCl)c2cc1OCc1ccc2ccccc2c1] | 5 |
| 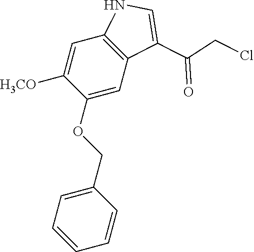 [SMILES: COc1cc2[nH]cc(C(=O)CCl)c2cc1OCc1ccccc1] | 4 |
| 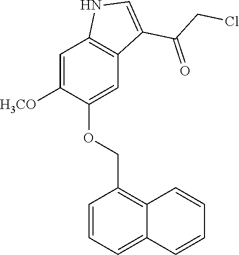 [SMILES: COc1cc2[nH]cc(C(=O)CCl)c2cc1OCc1cccc2ccccc12] | 6 |
| 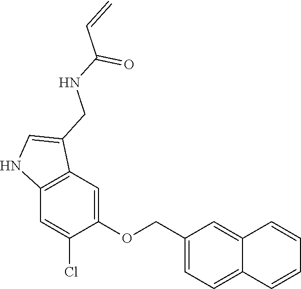 [SMILES: C=CC(=O)NCc1c[nH]c2cc(Cl)c(OCc3ccc4ccccc4c3)cc12] | 9 |
| 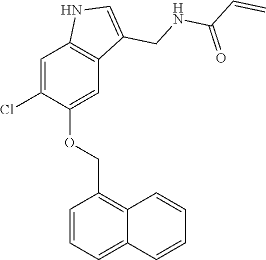 [SMILES: C=CC(=O)NCc1c[nH]c2cc(Cl)c(OCc3cccc4ccccc34)cc12] | 10 |
| 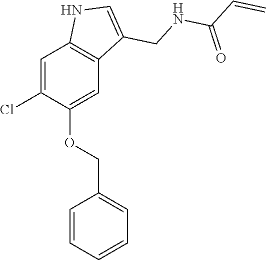 [SMILES: C=CC(=O)NCc1c[nH]c2cc(Cl)c(OCc3ccccc3)cc12] | 11 |
| 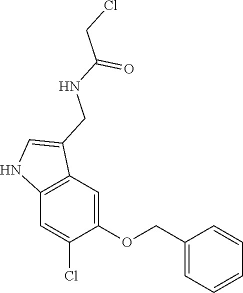 [SMILES: O=C(CCl)NCc1c[nH]c2cc(Cl)c(OCc3ccccc3)cc12] | 12 |
| 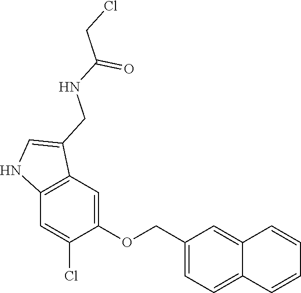 [SMILES: O=C(CCl)NCc1c[nH]c2cc(Cl)c(OCc3ccc4ccccc4c3)cc12] | 13 |
| 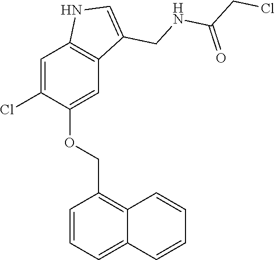 [SMILES: O=C(CCl)NCc1c[nH]c2cc(Cl)c(OCc3cccc4ccccc34)cc12] | 14 |
| 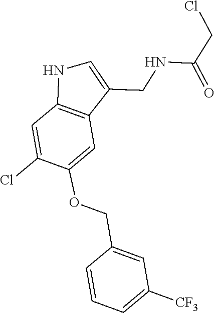 [SMILES: O=C(CCl)NCc1c[nH]c2cc(Cl)c(OCc3cccc(C(F)(F)F)c3)cc12] | 15 |
| 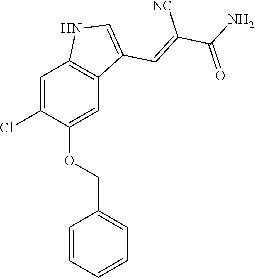 [SMILES: N#C/C(=C\c1c[nH]c2cc(Cl)c(OCc3ccccc3)cc12)C(N)=O] | 16 |
| 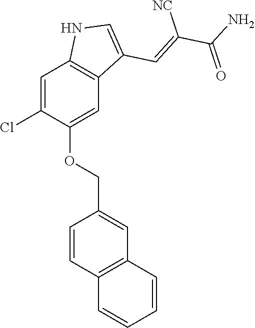 [SMILES: N#C/C(=C\c1c[nH]c2cc(Cl)c(OCc3ccc4ccccc4c3)cc12)C(N)=O] | 17 |
| 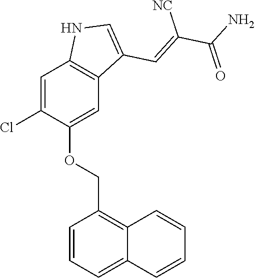 [SMILES: N#C/C(=C\c1c[nH]c2cc(Cl)c(OCc3cccc4ccccc34)cc12)C(N)=O] | 18 |
| 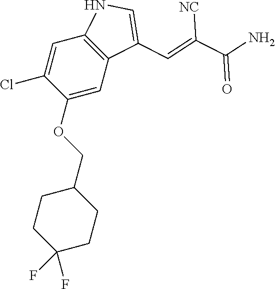 [SMILES: N#C/C(=C\c1c[nH]c2cc(Cl)c(OCC3CCC(F)(F)CC3)cc12)C(N)=O] | 19 |
| 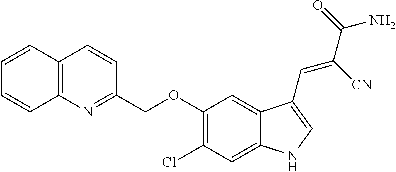 [SMILES: N#C/C(=C\c1c[nH]c2cc(Cl)c(OCc3ccc4ccccc4n3)cc12)C(N)=O] | 20 |
| 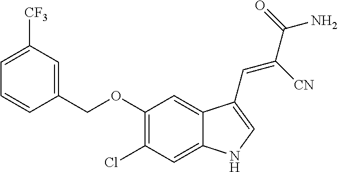 [SMILES: N#C/C(=C\c1c[nH]c2cc(Cl)c(OCc3cccc(C(F)(F)F)c3)cc12)C(N)=O] | 21 |
| 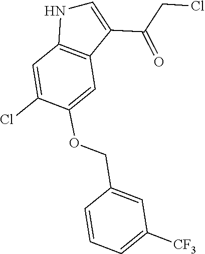 [SMILES: O=C(CCl)c1c[nH]c2cc(Cl)c(OCc3cccc(C(F)(F)F)c3)cc12] | 22 |
| 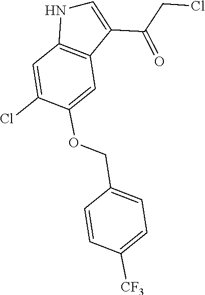 [SMILES: O=C(CCl)c1c[nH]c2cc(Cl)c(OCc3ccc(C(F)(F)F)cc3)cc12] | 23 |
| 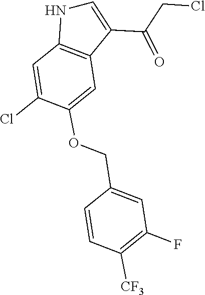 [SMILES: O=C(CCl)c1c[nH]c2cc(Cl)c(OCc3ccc(C(F)(F)F)c(F)c3)cc12] | 24 |
| 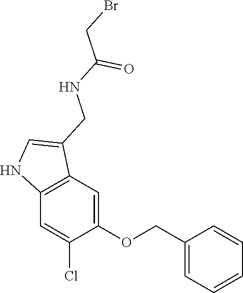 [SMILES: O=C(CBr)NCc1c[nH]c2cc(Cl)c(OCc3ccccc3)cc12] | CV-4-21 |
| 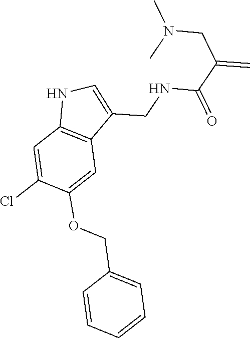 [SMILES: C=C(CN(C)C)C(=O)NCc1c[nH]c2cc(Cl)c(OCc3ccccc3)cc12] | 25 |

or a salt, solvate, hydrate, polymorph, co-crystal, tautomer, stereoisomer, isotopically labeled compound, or prodrug thereof.

In some embodiments, the compound of Formula I, or a salt, solvate, hydrate, polymorph, co-crystal, tautomer, stereoisomer, isotopically labeled compound, or prodrug thereof, wherein the compound is not any one of the formulae shown in Table 1. In some embodiments, the compound of Formula I, or a salt, solvate, hydrate, polymorph, co-crystal, tautomer, stereoisomer, isotopically labeled compound, or prodrug thereof, wherein the compound is not of Formula I-A.

In another aspect, provided herein is a compound of formula 7:

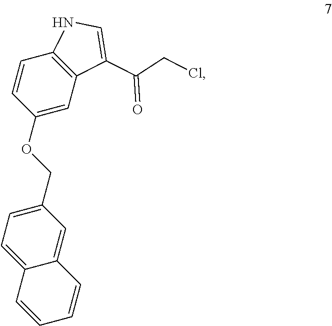 [SMILES: O=C(CCl)c1c[nH]c2ccc(OCc3ccc4ccccc4c3)cc12]

or a salt, solvate, hydrate, polymorph, co-crystal, tautomer, stereoisomer, isotopically labeled compound, or prodrug thereof.

In another aspect, provided herein is a compound of formula 12:

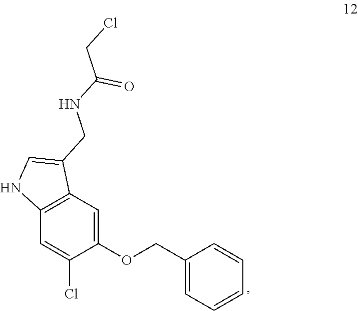 [SMILES: O=C(CCl)NCc1c[nH]c2cc(Cl)c(OCc3ccccc3)cc12]

or a salt, solvate, hydrate, polymorph, co-crystal, tautomer, stereoisomer, isotopically labeled compound, or prodrug thereof.

In another aspect, provided herein is a compound of formula 22:

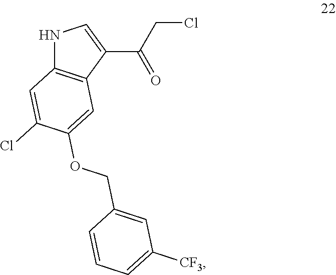 [SMILES: O=C(CCl)c1c[nH]c2cc(Cl)c(OCc3cccc(C(F)(F)F)c3)cc12]

or a salt, solvate, hydrate, polymorph, co-crystal, tautomer, stereoisomer, isotopically labeled compound, or prodrug thereof.

“A compound of the present disclosure” or “a compound provided herein” refers to a compound of Formulae II, I, I-A, I-B, I-C, or shown in Table 1, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula II, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula I, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula II, or a pharmaceutically acceptable salt or tautomer thereof. In some embodiments, a compound of the present disclosure is a compound of Formula II, or a pharmaceutically acceptable salt or stereoisomer thereof. In some embodiments, a compound of the present disclosure is a compound of Formula II, or a pharmaceutically acceptable salt thereof. In some embodiments, a compound of the present disclosure is a compound of Formula I, or a pharmaceutically acceptable salt or tautomer thereof. In some embodiments, a compound of the present disclosure is a compound of Formula I, or a pharmaceutically acceptable salt or stereoisomer thereof. In some embodiments, a compound of the present disclosure is a compound of Formula I, or a pharmaceutically acceptable salt thereof. In some embodiments, a compound of the present disclosure is a compound shown in Table 1, or a pharmaceutically acceptable salt, solvate, hydrate, polymorph, co-crystal, tautomer, stereoisomer, isotopically labeled compound, or prodrug thereof. In some embodiments, a compound of the present disclosure is a compound shown in Table 1, or a pharmaceutically acceptable salt or tautomer thereof. In some embodiments, a compound of the present disclosure is a compound shown in Table 1, or a pharmaceutically acceptable salt or stereoisomer thereof. In some embodiments, a compound of the present disclosure is a compound shown in Table 1, or a pharmaceutically acceptable salt thereof.

### Pharmaceutical Compositions and Kits

In another aspect, provided herein is a pharmaceutical composition comprising a compound provided herein, or a salt, solvate, hydrate, polymorph, co-crystal, tautomer, stereoisomer, isotopically labeled compound, or prodrug thereof; and optionally a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease in a subject in need thereof. In certain embodiments, the effective amount is an amount effective for inhibiting the activity (e.g., aberrant activity, such as increased activity) of TEAD in a subject or cell.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal.

In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

In certain embodiments, the effective amount is an amount effective for inhibiting the activity of TEAD by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of TEAD by at least 10%, at least 20%, at least 30%, at least 40%, at least 50%, at least 60%, at least 70%, at least 80%, at least 90%, at least 95%, or at least 98%.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents or fillers, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents or fillers include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, starches (such as dry starch, cornstarch), sugars (such as powdered sugar), calcium trisulfate, carboxymethylcellulose calcium, dextrate, dextrin, dextrose, fructose, lactitol, lactose, magnesium carbonate, magnesium, maltitol, maltodextrin, maltose, sucrose, glucose, mannitol, silicic acid, xylitol, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary disintegrating agents or disintegrants include agar, algin, alginic acid, sodium alginate, silicates, sodium carbonate, calcium carbonate, carboxymethylcellulose, cellulose, clay, colloidal silicon dioxide, croscarmellose sodium, crospovidone, rubber, magnesium silicate, methylcellulose, potassium krillin, hydroxypropylcellulose (e.g., low substituted Hydroxypropylcellulose), crosslinked polyvinylpyrrolidone, hydroxypropylcellulose, and starch (e.g., sodium glycolate starch, potato or tapioca starch).

Exemplary binding agents include starch (e.g., glycolate starch, cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip©, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include agar, ethyl oleate, ethyl laurate, glycerin, blyceryl palmitostearate, magnesium oxide, magnesium stearate, mannitol, poloxamer, glycol, sodium stearyl, sorbitol, zinc stearate, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

In some embodiments, injectable preparations of the compositions disclosed herein are in the form of a ready-to-use (“RTU”) preparation that can be directly administered to a subject. In some embodiments, the RTU preparation is a suspension. In some embodiments, the RTU preparation is a solution. In some embodiments, the RTU preparation is an emulsion. In some embodiments, injectable preparations of the compositions disclosed herein are in the form of a solid that is reconstituted prior to administration. In some embodiments, the solid is a lyophilized solid. In some embodiments, injectable preparations of the compositions disclosed herein are in the form of a liquid or suspension that is diluted prior to administration.

In some embodiments, the pharmaceutical compositions disclosed herein comprise a bulking agent. Bulking agents can be used, e.g., to improve the appearance of a solid composition, to provide visible “bulk” to demonstrate product quality or to facilitate preparation, e.g., of a solid composition prepared for reconstitution prior to administration.

Bulking agents can be used for low dose (high potency) drugs that do not have the necessary bulk to support their own structure or provide a visible composition in a unit dosage form.

Bulking agents are used in lyophilized formulations. Bulking agents provide a desirable structure for a lyophilized cake comprising pores that provide the means for vapor to escape from the product during lyophilization cycles, and facilitate dissolution on reconstitution. In some embodiments, the bulking agent is mannitol, lactose, sucrose, dextran, trehalose, povidone, dextran, glycine, isoleucine, methionine, or a cyclodextrin (e.g., (2-hydroxypropyl)-β-cyclodextrin).

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmaceutics. They may optionally comprise opacifying agents and can be of a composition that they release the active ingredient(s) only, or preferentially, in a certain part of the intestinal tract, optionally, in a delayed manner.

Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner.

Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions.

Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension.

Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject. In some embodiments, the compound or pharmaceutical composition provided herein is formulated for oral administration, intraperitoneal injection, or subcutaneous injection. In some embodiments, the compound or pharmaceutical composition provided herein is formulated for oral administration. In some embodiments, the compound or pharmaceutical composition provided herein is formulated for intraperitoneal injection. In some embodiments, the compound or pharmaceutical composition provided herein is formulated for subcutaneous injection.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of TEAD in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In some embodiments, the pharmaceutical composition further comprises an additional pharmaceutical agent. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents.

Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease. Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of TEAD. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In some embodiments, the additional pharmaceutical agent is a kinase inhibitor. In some embodiments, the additional pharmaceutical agent is atezolizumab-bevacizumab combination, sorafenib, or lenvatinib. In some embodiments the additional pharmaceutical agent is sorafenib.

Also encompassed by the disclosure are kits (e.g., pharmaceutical packs). In another aspect, provided herein is a kit comprising: a compound provided herein, or a salt, solvate, hydrate, polymorph, co-crystal, tautomer, stereoisomer, isotopically labeled compound, or prodrug thereof, or a pharmaceutical composition provided herein; and instructions for using the compound, or salt, solvate, hydrate, polymorph, co-crystal, tautomer, stereoisomer, isotopically labeled compound, or prodrug thereof, or pharmaceutical composition.

In some embodiments, the kits provided herein comprise a pharmaceutical composition or compound described herein and a container (e.g., a vial, ampule, bottle, syringe, and/or dispenser package, or other suitable container). In some embodiments, provided kits optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In some embodiments, the kit includes a first container comprising a compound or pharmaceutical composition described herein. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof. In certain embodiments, the kits are useful for inhibiting the activity (e.g., aberrant activity, such as increased activity) of TEAD in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. In certain embodiments, the kits and instructions provide for inhibiting the activity (e.g., aberrant activity, such as increased activity) of TEAD in a subject or cell. In some embodiments, a kit described herein includes one or more additional pharmaceutical agents described herein as a separate composition.

### Methods and Uses

In another aspect, the invention provides a method of treating a subject suffering from or susceptible to a disorder or disease identified herein, wherein the subject has been identified as in need of treatment for the disorder or disease, comprising administering to said subject in need thereof, an effective amount of a compound or pharmaceutical composition herein, such that said subject is treated for said disorder or disease.

In another aspect, provided herein is a method of treating a disease in a subject in need thereof, the method comprising administering to the subject an effective amount of: a compound provided herein, or salt, solvate, hydrate, polymorph, co-crystal, tautomer, stereoisomer, isotopically labeled compound, or prodrug thereof; or a pharmaceutical composition provided herein.

In another aspect, provided herein is a method of preventing a disease in a subject in need thereof, the method comprising administering to the subject an effective amount of: a compound provided herein, or salt, solvate, hydrate, polymorph, co-crystal, tautomer, stereoisomer, isotopically labeled compound, or prodrug thereof; or a pharmaceutical composition provided herein.

Methods delineated herein include those wherein the subject is identified as in need of a particular stated treatment. Identifying a subject in need of such treatment can be in the judgment of a subject or a health care professional and can be subjective (e.g., opinion) or objective (e.g., measurable by a test or diagnostic method, including those delineated herein).

Another object of the present invention is the use of a compound as described herein (e.g., of any formulae herein) in the manufacture of a medicament for use in the treatment of a disorder or disease delineated herein. Another object of the present invention is the use of a compound as described herein (e.g., of any formulae herein) for use in the treatment of a disorder or disease delineated herein. Another object of the present invention is the use of a compound as described herein (e.g., of any formulae herein) in the manufacture of composition for use in the treatment or prevention of a disorder or disease delineated herein.

In some embodiments, the disease is a proliferative disease. In some embodiments, the disease is cancer. In some embodiments, the cancer is breast, pancreatic, liver, colorectal, lung, ovarian, or prostate cancer. In some embodiments, the cancer is breast cancer. In some embodiments, the cancer is pancreatic cancer. In some embodiments, the cancer is liver cancer. In some embodiments, the cancer is colorectal cancer. In some embodiments, the cancer is lung cancer. In some embodiments, the cancer is ovarian cancer. In some embodiments, the cancer is prostate cancer. In some embodiments, the cancer is hepatocellular carcinoma, triple negative breast cancer, mesothelioma, or hepatoblastoma. In some embodiments, the cancer is hepatocellular carcinoma. In some embodiments, the cancer is triple negative breast cancer. In some embodiments, the cancer is mesothelioma. In some embodiments, the cancer is hepatoblastoma.

In another aspect, provided herein is a method of inhibiting the activity of TEAD in a subject in need thereof, the method comprising administering to the subject an effective amount of: a compound provided herein, or salt, solvate, hydrate, polymorph, co-crystal, tautomer, stereoisomer, isotopically labeled compound, or prodrug thereof; or a pharmaceutical composition provided herein.

In another aspect, provided herein is a method of inhibiting the activity of TEAD in a cell, tissue, or biological sample the method comprising contacting the cell, tissue, or biological sample with an effective amount of: a compound provided herein, or salt, solvate, hydrate, polymorph, co-crystal, tautomer, stereoisomer, isotopically labeled compound, or prodrug thereof; or a pharmaceutical composition provided herein.

In some embodiments, the effective amount is effective in inhibiting TEAD. In some embodiments, the effective amount is effective in inhibiting TEAD transcription activity. In some embodiments, the effective amount is about 1 mg/kg to about 200 mg/kg. In some embodiments, the effective amount is about 1 mg/kg to about 150 mg/kg. In some embodiments, the effective amount is about 1 mg/kg to about 100 mg/kg. In some embodiments, the effective amount is about 1 mg/kg to about 75 mg/kg. In some embodiments, the effective amount is about 1 mg/kg to about 70 mg/kg. In some embodiments, the effective amount is about 1 mg/kg to about 60 mg/kg. In some embodiments, the effective amount is about 1 mg/kg to about 55 mg/kg. In some embodiments, the effective amount is about 10 mg/kg to about 200 mg/kg. In some embodiments, the effective amount is about 10 mg/kg to about 150 mg/kg. In some embodiments, the effective amount is about 10 mg/kg to about 100 mg/kg. In some embodiments, the effective amount is about 10 mg/kg to about 75 mg/kg. In some embodiments, the effective amount is about 10 mg/kg to about 70 mg/kg. In some embodiments, the effective amount is about 10 mg/kg to about 60 mg/kg. In some embodiments, the effective amount is about 10 mg/kg to about 55 mg/kg. In some embodiments, the effective amount is about 20 mg/kg to about 200 mg/kg. In some embodiments, the effective amount is about 20 mg/kg to about 150 mg/kg. In some embodiments, the effective amount is about 20 mg/kg to about 100 mg/kg. In some embodiments, the effective amount is about 20 mg/kg to about 75 mg/kg. In some embodiments, the effective amount is about 20 mg/kg to about 70 mg/kg. In some embodiments, the effective amount is about 20 mg/kg to about 60 mg/kg. In some embodiments, the effective amount is about 20 mg/kg to about 55 mg/kg. In some embodiments, the effective amount is about 10 mg/kg, about 15 mg/kg, about 20 mg/kg about 25 mg/kg, about 30 mg/kg, about 35 mg/kg, about 40 mg/kg, about 45 mg/kg, about 50 mg/kg, or about 55 mg/kg. In some embodiments, the effective amount is about 25 mg/kg. In some embodiments, the effective amount is about 50 mg/kg.

In some embodiments, the method further comprises co-administering an additional pharmaceutical agent. In some embodiments, the additional pharmaceutical agent is co-administered on the same schedule as the compound provided herein, or salt, solvate, hydrate, polymorph, co-crystal, tautomer, stereoisomer, isotopically labeled compound, or prodrug thereof; or as the pharmaceutical composition provided herein. In some embodiments, the co-administration increases sensitivity to the additional pharmaceutical agent. In some embodiments, the co-administration increases sensitivity to the compound provided herein, or salt, solvate, hydrate, polymorph, co-crystal, tautomer, stereoisomer, isotopically labeled compound, or prodrug thereof; or to the pharmaceutical composition provided herein. In some embodiments, the additional pharmaceutical agent is sorafenib.

In some embodiments, the additional pharmaceutical agent is administered in the amount of about 1 mg/kg to about 200 mg/kg. In some embodiments, the additional pharmaceutical agent is administered in the amount of about 10 mg/kg to about 200 mg/kg. In some embodiments, the additional pharmaceutical agent is administered in the amount of about 20 mg/kg to about 200 mg/kg. In some embodiments, the additional pharmaceutical agent is administered in the amount of about 50 mg/kg to about 200 mg/kg. In some embodiments, the additional pharmaceutical agent is administered in the amount of about 1 mg/kg to about 150 mg/kg. In some embodiments, the additional pharmaceutical agent is administered in the amount of about 10 mg/kg to about 150 mg/kg. In some embodiments, the additional pharmaceutical agent is administered in the amount of about 20 mg/kg to about 150 mg/kg. In some embodiments, the additional pharmaceutical agent is administered in the amount of about 50 mg/kg to about 150 mg/kg. In some embodiments, the additional pharmaceutical agent is administered in the amount of about 80 mg/kg. In some embodiments, the additional pharmaceutical agent is administered in the amount of about 90 mg/kg. In some embodiments, the additional pharmaceutical agent is administered in the amount of about 100 mg/kg. In some embodiments, the additional pharmaceutical agent is administered in the amount of about 110 mg/kg. In some embodiments, the additional pharmaceutical agent is administered in the amount of about 120 mg/kg.

In certain embodiments, the subject is an animal. The animal may be of either sex and may be at any stage of development. In certain embodiments, the subject described herein is a human. In some embodiments, the subject is a human aged 18 and older. In some embodiments, the subject is a human under age 18. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell, tissue, or biological sample is in vitro. In certain embodiments, the cell, tissue, or biological sample is in vivo.

Another aspect of the invention is a method of making a compound of any of the formulae herein comprising one or more of the chemical reaction transformations described herein. In some embodiments, the method is performed according to an example provided herein.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof. In some embodiments, a salt of a compound provided herein is a pharmaceutically acceptable salt.

### EMBODIMENTS LISTING

1. A compound of Formula II:

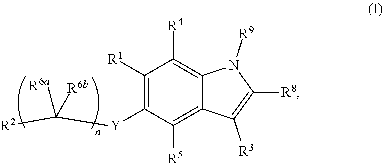

or a salt, solvate, hydrate, polymorph, co-crystal, tautomer, stereoisomer, isotopically labeled compound, or prodrug thereof, wherein:
Y is —NR^{A}—, —O—, —S—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A}.
R² is optionally substituted C₁₋₆ alkyl, —OR^{A}, optionally substituted carbocyclyl, optionally substituted heterocyclyl, optionally substituted aryl, or optionally substituted heteroaryl;
R³ is

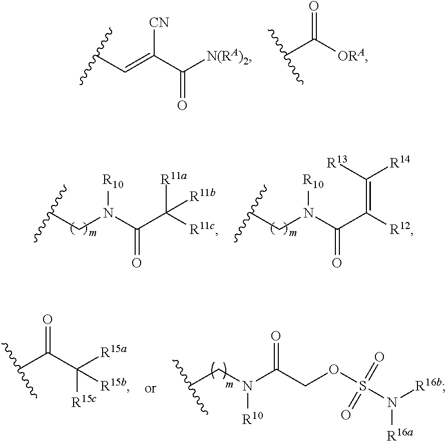

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each of R^{16a} and R^{16b} is independently hydrogen, optionally substituted C₁₋₆ alkyl, or optionally substituted aryl;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

2. The compound of embodiment 1, or a salt, solvate, hydrate, polymorph, co-crystal, tautomer, stereoisomer, isotopically labeled compound, or prodrug thereof, wherein the compound is of Formula I:

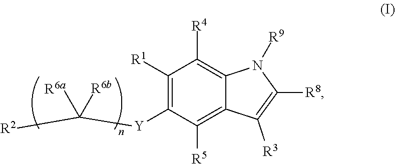

wherein:
Y is —NR^{A}—, —O—, —S—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A}.
R² is optionally substituted C₁₋₆ alkyl, —OR^{A}, optionally substituted carbocyclyl, optionally substituted heterocyclyl, optionally substituted aryl, or optionally substituted heteroaryl;
R³ is

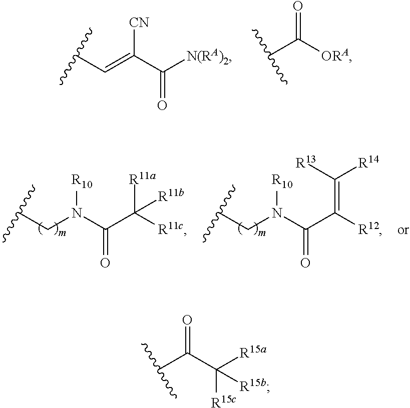

R⁴ is hydrogen or halogen;
R⁵ is hydrogen or halogen;
each of R^{6a} and R^{6b} is independently hydrogen, halogen, optionally substituted alkyl;
each of R^{7a} and R^{7b} is independently hydrogen, halogen, optionally substituted alkyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

3. The compound of any preceding embodiment, or a salt, solvate, hydrate, polymorph, co-crystal, tautomer, stereoisomer, isotopically labeled compound, or prodrug thereof, wherein the compound is of Formula I-A:

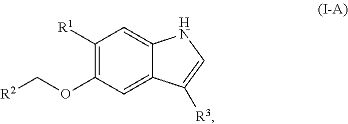

wherein:
R¹ is —Cl or —OMe;
R² is

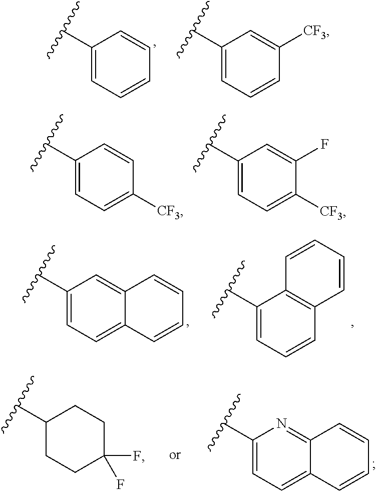

and
R³ is

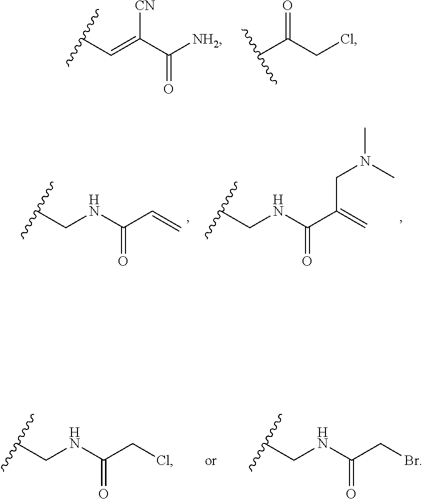

4. The compound of any preceding embodiment, or a salt, solvate, hydrate, polymorph, co-crystal, tautomer, stereoisomer, isotopically labeled compound, or prodrug thereof, wherein the compound is of Formula I-B:

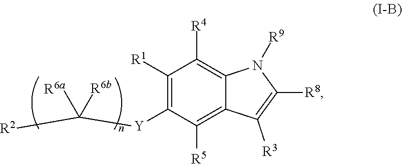

wherein:
Y is —NR^{A}—, —O—, or —CR^{7a}R^{7b}—;
R¹ is —F, —Br, —I, optionally substituted C₁₋₆ alkyl, optionally substituted carbocyclyl, —CN, —OR^{B}, —C(═O)OR^{A}, —Si(R^{A})₃, or —S(═O)₂R^{A}.
R² is optionally substituted C₁₋₆ alkyl, —OR^{A}, optionally substituted carbocyclyl, optionally substituted heterocyclyl, optionally substituted aryl, or optionally substituted heteroaryl; R³ is

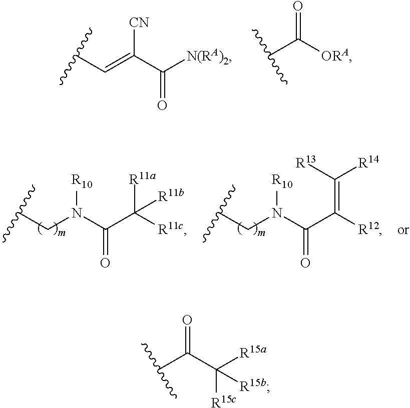

R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{B} is independently hydrogen, optionally substituted C₂-C₁₀ alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

5. The compound of any preceding embodiment, or a salt, solvate, hydrate, polymorph, co-crystal, tautomer, stereoisomer, isotopically labeled compound, or prodrug thereof, wherein the compound is of Formula I-C:

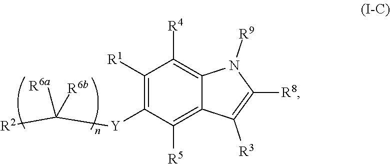

wherein:
Y is —NR^{A}—, —O—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A}.
R² is optionally substituted C₁₋₆ alkyl, —OR^{A}, optionally substituted carbocyclyl, optionally substituted heterocyclyl, optionally substituted aryl, or optionally substituted heteroaryl;
R³ is

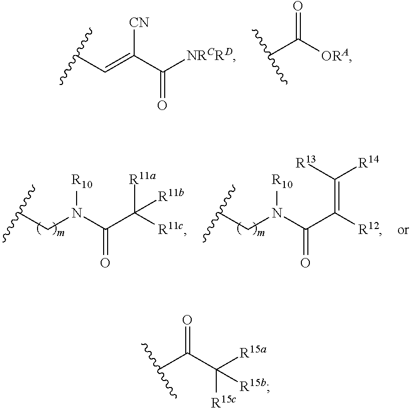

R⁴ is hydrogen or halogen;
R⁴ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen, —F, or —I;
R¹² is independently hydrogen, halogen, or —CN;
each of R¹³ and R¹⁴ is independently hydrogen, halogen, optionally substituted C₁₋₆ alkyl, optionally substituted C₁₋₆ heteroalkyl, or —CN;
provided that at least one of R¹², R¹³, and R¹⁴ is halogen, optionally substituted C₁₋₆ alkyl, optionally substituted C₁₋₆ heteroalkyl, or —CN;
each of R^{15a}, R^{15b}, and R^{15c}, is independently hydrogen, —F, —Br, or —I, provided that at least one of R^{15a}, R^{15b}, and R^{15c} is —F, —Br, or —I;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{C} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring;
R^{D} is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring;
n is an integer from 0 to 1; and
m is an integer from 0 to 1.

6. The compound of any preceding embodiment, or a salt, solvate, hydrate, polymorph, co-crystal, tautomer, stereoisomer, isotopically labeled compound, or prodrug thereof, wherein Y is —NH—, —O—, —S—, or —CH₂-.

7. The compound of any preceding embodiment, or a salt, solvate, hydrate, polymorph, co-crystal, tautomer, stereoisomer, isotopically labeled compound, or prodrug thereof, wherein Y is —O—.

8. The compound of any preceding embodiment, or a salt, solvate, hydrate, polymorph, co-crystal, tautomer, stereoisomer, isotopically labeled compound, or prodrug thereof, wherein Y is —NH—, —NMe—, —S—, or —CH₂-.

9. The compound of any preceding embodiment, or a salt, solvate, hydrate, polymorph, co-crystal, tautomer, stereoisomer, isotopically labeled compound, or prodrug thereof, wherein R¹ is halogen, —CN, C₁₋₄ alkyl optionally substituted with one or more fluorine atoms, cyclopropyl, —C(═O)O(C₁₋₃ alkyl), —O(C₁₋₃ alkyl optionally substituted with one or more fluorine atoms), —Si(C₁₋₃ alkyl)₃, or —S(═O)₂(C₁₋₃ alkyl).

10. The compound of any preceding embodiment, or a salt, solvate, hydrate, polymorph, co-crystal, tautomer, stereoisomer, isotopically labeled compound, or prodrug thereof, wherein R¹ is halogen or —O(C₁₋₃ alkyl optionally substituted with one or more fluorine atoms).

11. The compound of any preceding embodiment, or a salt, solvate, hydrate, polymorph, co-crystal, tautomer, stereoisomer, isotopically labeled compound, or prodrug thereof, wherein R¹ is —Cl.

12. The compound of any preceding embodiment, or a salt, solvate, hydrate, polymorph, co-crystal, tautomer, stereoisomer, isotopically labeled compound, or prodrug thereof, wherein R¹ is halogen or —OMe.

13. The compound of any preceding embodiment, or a salt, solvate, hydrate, polymorph, co-crystal, tautomer, stereoisomer, isotopically labeled compound, or prodrug thereof, wherein R¹ is —F, —Br, or —I.

14. The compound of any preceding embodiment, or a salt, solvate, hydrate, polymorph, co-crystal, tautomer, stereoisomer, isotopically labeled compound, or prodrug thereof, wherein R¹ is —O(C₂₋₃ alkyl optionally substituted with one or more fluorine atoms).

15. The compound of any preceding embodiment, or a salt, solvate, hydrate, polymorph, co-crystal, tautomer, stereoisomer, isotopically labeled compound, or prodrug thereof, wherein R¹ is —F, —Br, —I, —CN, C₁₋₄ alkyl optionally substituted with one or more fluorine atoms, cyclopropyl, —C(═O)O(C₁₋₃ alkyl), —O(C₂₋₃ alkyl optionally substituted with one or more fluorine atoms), —Si(C₁₋₃ alkyl)₃, or —S(═O)₂(C₁₋₃ alkyl).

16. The compound of any preceding embodiment, or a salt, solvate, hydrate, polymorph, co-crystal, tautomer, stereoisomer, isotopically labeled compound, or prodrug thereof, wherein R¹ is —CN, C₁₋₄ alkyl optionally substituted with one or more fluorine atoms, cyclopropyl, —C(═O)O(C₁₋₃ alkyl), —Si(C₁₋₃ alkyl)₃, or —S(═O)₂(C₁₋₃ alkyl).

17. The compound of any preceding embodiment, or a salt, solvate, hydrate, polymorph, co-crystal, tautomer, stereoisomer, isotopically labeled compound, or prodrug thereof, wherein R² is C₁₋₆ alkyl optionally substituted with one or more fluorine atoms, —O(C₁₋₅ alkyl optionally substituted with one or more fluorine atoms), optionally substituted C₆₋₁₀ aryl, optionally substituted 5-10-membered heteroaryl, optionally substituted C₄₋₈ cycloalkyl, or optionally substituted 3-7-membered heterocyclyl.

18. The compound of any preceding embodiment, or a salt, solvate, hydrate, polymorph, co-crystal, tautomer, stereoisomer, isotopically labeled compound, or prodrug thereof, wherein R² is phenyl, wherein the phenyl is optionally substituted by one or more instances of halogen; C₁₋₄ alkyl optionally substituted with one or more fluorine atoms; C₃₋₆ cycloalkyl; —O(C₁₋₄ alkyl optionally substituted with one or more fluorine atoms); pentafluorosulfanyl; —CN; —Si(C₁₋₃ alkyl); —S(═O)₂(C₁₋₃ alkyl); phenyl optionally substituted by one or more —CF₃ groups; —CO₂(C₁₋₄ alkyl); or —S(C₁₋₄alkyl).

19. The compound of any preceding embodiment, or a salt, solvate, hydrate, polymorph, co-crystal, tautomer, stereoisomer, isotopically labeled compound, or prodrug thereof, wherein R² is C₄₋₈ cycloalkyl optionally substituted by halogen or C₁₋₄ alkyl optionally substituted with one or more fluorine atoms.

20. The compound of any preceding embodiment, or a salt, solvate, hydrate, polymorph, co-crystal, tautomer, stereoisomer, isotopically labeled compound, or prodrug thereof, wherein R² is

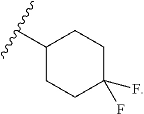 [SMILES: *C1CCC(F)(F)CC1]

21. The compound of any preceding embodiment, or a salt, solvate, hydrate, polymorph, co-crystal, tautomer, stereoisomer, isotopically labeled compound, or prodrug thereof, wherein R² is 3-7-membered heterocyclyl optionally substituted by halogen, C₁₋₄ fluoroalkyl, or phenyl optionally substituted by one or more fluorine atoms or —CF₃ groups.

22. The compound of any preceding embodiment, or a salt, solvate, hydrate, polymorph, co-crystal, tautomer, stereoisomer, isotopically labeled compound, or prodrug thereof, wherein R² is optionally substituted phenyl or optionally substituted naphthyl.

23. The compound of any preceding embodiment, or a salt, solvate, hydrate, polymorph, co-crystal, tautomer, stereoisomer, isotopically labeled compound, or prodrug thereof, wherein R² is phenyl optionally substituted either with halogen or with C₁₋₄ alkyl optionally substituted with one or more halogen atoms.

24. The compound of any preceding embodiment, or a salt, solvate, hydrate, polymorph, co-crystal, tautomer, stereoisomer, isotopically labeled compound, or prodrug thereof, wherein R² is phenyl optionally substituted with —F, —CF₃, —CHF₂, —CH₂F, or —Me.

25. The compound of any preceding embodiment, or a salt, solvate, hydrate, polymorph, co-crystal, tautomer, stereoisomer, isotopically labeled compound, or prodrug thereof, wherein R² is naphthyl optionally substituted either with halogen or with C₁₋₄ alkyl optionally substituted with one or more halogen atoms.

26. The compound of any preceding embodiment, or a salt, solvate, hydrate, polymorph, co-crystal, tautomer, stereoisomer, isotopically labeled compound, or prodrug thereof, wherein R² is naphthyl optionally substituted with —F, —CF₃, —CHF₂, —CH₂F, or —Me.

27. The compound of any preceding embodiment, or a salt, solvate, hydrate, polymorph, co-crystal, tautomer, stereoisomer, isotopically labeled compound, or prodrug thereof, wherein R² is

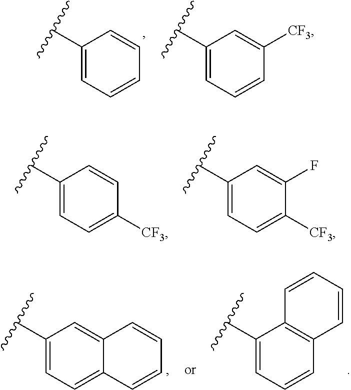

28. The compound of any preceding embodiment, or a salt, solvate, hydrate, polymorph, co-crystal, tautomer, stereoisomer, isotopically labeled compound, or prodrug thereof, wherein R² is

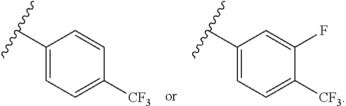 [SMILES: *c1ccc(C(F)(F)F)cc1 ; "or" ; *c1ccc(C(F)(F)F)c(F)c1]

29. The compound of any preceding embodiment, or a salt, solvate, hydrate, polymorph, co-crystal, tautomer, stereoisomer, isotopically labeled compound, or prodrug thereof, wherein R³ is

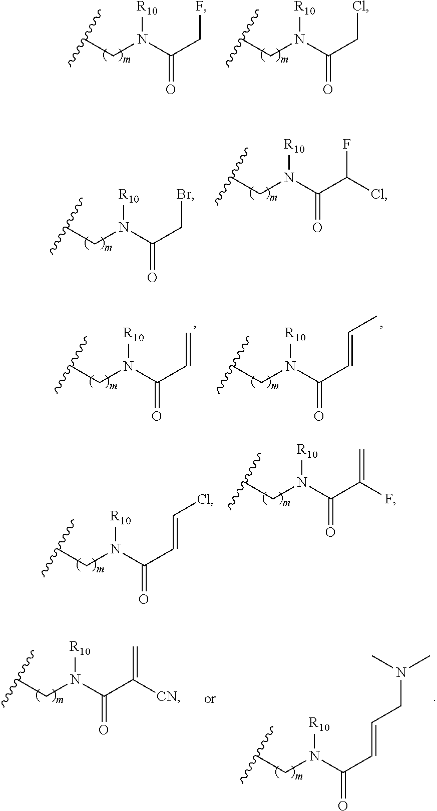

30. The compound of any preceding embodiment, or a salt, solvate, hydrate, polymorph, co-crystal, tautomer, stereoisomer, isotopically labeled compound, or prodrug thereof, wherein R³ is

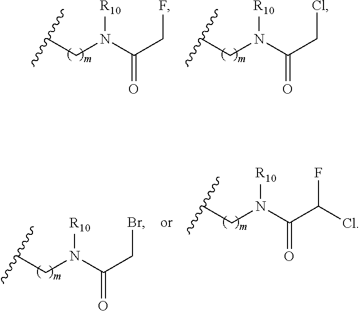

31. The compound of any preceding embodiment, or a salt, solvate, hydrate, polymorph, co-crystal, tautomer, stereoisomer, isotopically labeled compound, or prodrug thereof, wherein R³ is

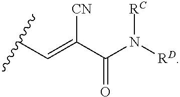

32. The compound of any preceding embodiment, or a salt, solvate, hydrate, polymorph, co-crystal, tautomer, stereoisomer, isotopicaly labeled compound, or prodrug thereof, wherein R³ is

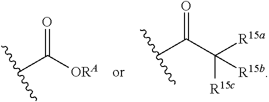

33. The compound of any preceding embodiment, or a salt, solvate, hydrate, polymorph, co-crystal, tautomer, stereoisomer, isotopically labeled compound, or prodrug thereof, wherein R³ is

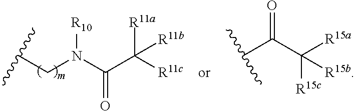

34. The compound of any preceding embodiment, wherein R³ is

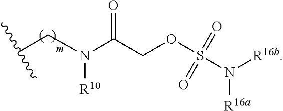

35. The compound of any preceding embodiment, wherein R³ is

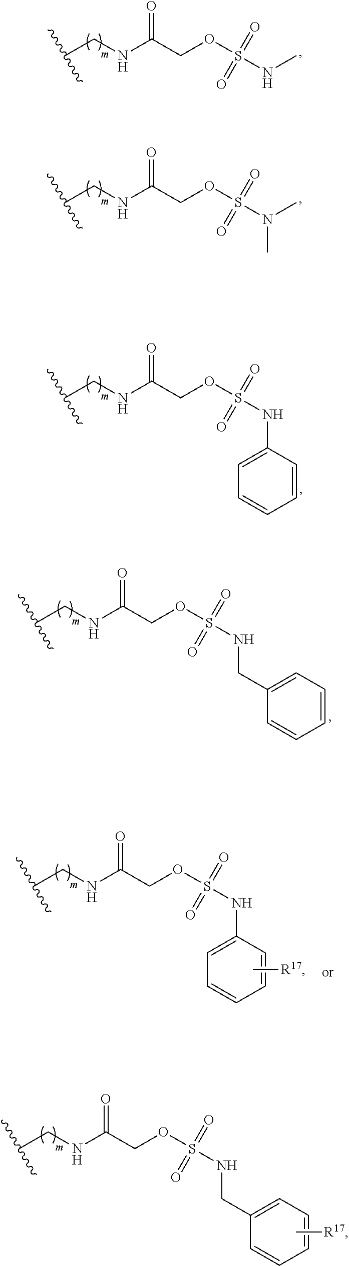

wherein R¹⁷ is halogen, —CN, optionally substituted C₁₋₂ alkyl, —OH, —O(optionally substituted C₁₋₂ alkyl), —C(═O)O(optionally substituted C₁₋₂ alkyl), or —C(═O)NH₂.

36. The compound of any preceding embodiment, or a salt, solvate, hydrate, polymorph, co-crystal, tautomer, stereoisomer, isotopically labeled compound, or prodrug thereof, wherein R³ is

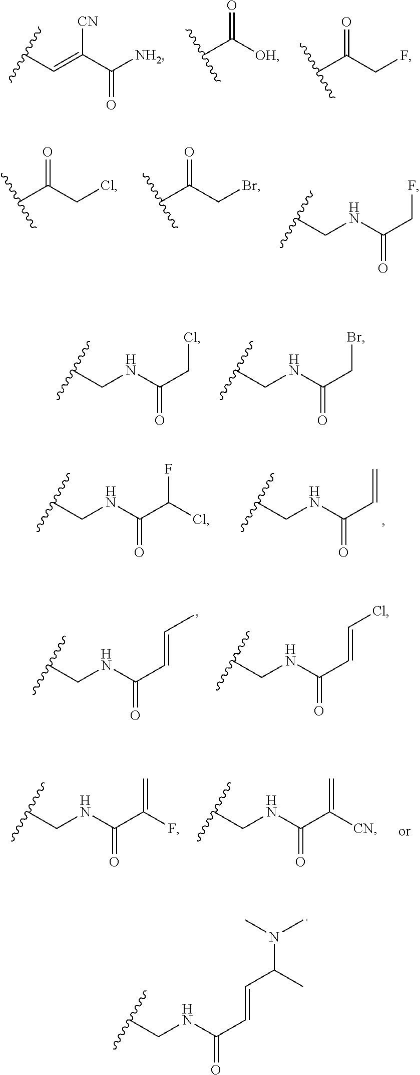

37. The compound of any preceding embodiment, or a salt, solvate, hydrate, polymorph, co-crystal, tautomer, stereoisomer, isotopically labeled compound, or prodrug thereof, wherein R³ is

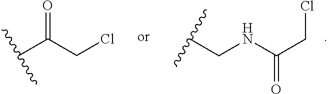 [SMILES: *C(=O)CCl ; *CNC(=O)CCl]

38. The compound of any preceding embodiment, or a salt, solvate, hydrate, polymorph, co-crystal, tautomer, stereoisomer, isotopically labeled compound, or prodrug thereof, wherein R⁴ is hydrogen.

39. The compound of any preceding embodiment, or a salt, solvate, hydrate, polymorph, co-crystal, tautomer, stereoisomer, isotopically labeled compound, or prodrug thereof, wherein R⁴ is fluorine.

40. The compound of any preceding embodiment, or a salt, solvate, hydrate, polymorph, co-crystal, tautomer, stereoisomer, isotopically labeled compound, or prodrug thereof, wherein R⁵ is hydrogen.

41. The compound of any preceding embodiment, or a salt, solvate, hydrate, polymorph, co-crystal, tautomer, stereoisomer, isotopically labeled compound, or prodrug thereof, wherein R⁵ is fluorine.

42. The compound of any preceding embodiment, or a salt, solvate, hydrate, polymorph, co-crystal, tautomer, stereoisomer, isotopically labeled compound, or prodrug thereof, wherein R^{6a} and R^{6b} are each independently hydrogen, halogen, or C₁₋₃ alkyl.

43. The compound of any preceding embodiment, or a salt, solvate, hydrate, polymorph, co-crystal, tautomer, stereoisomer, isotopically labeled compound, or prodrug thereof, wherein R^{6a} and R^{6b} are each hydrogen.

44. The compound of any preceding embodiment, or a salt, solvate, hydrate, polymorph, co-crystal, tautomer, stereoisomer, isotopically labeled compound, or prodrug thereof, wherein R^{7a} and R^{7b} are each independently hydrogen, halogen, or C₁₋₃ alkyl.

45. The compound of any preceding embodiment, or a salt, solvate, hydrate, polymorph, co-crystal, tautomer, stereoisomer, isotopically labeled compound, or prodrug thereof, wherein R^{7a} and R^{7b} are each hydrogen.

46. The compound of any preceding embodiment, or a salt, solvate, hydrate, polymorph, co-crystal, tautomer, stereoisomer, isotopically labeled compound, or prodrug thereof, wherein R⁸ is hydrogen, C₁₋₃ alkyl, —CN, —OH, —O(C₁₋₃ alkyl), —C(═O)O(C₁₋₂ alkyl), or —CONH₂.

47. The compound of any preceding embodiment, or a salt, solvate, hydrate, polymorph, co-crystal, tautomer, stereoisomer, isotopically labeled compound, or prodrug thereof, wherein R⁸ is hydrogen.

48. The compound of any preceding embodiment, or a salt, solvate, hydrate, polymorph, co-crystal, tautomer, stereoisomer, isotopically labeled compound, or prodrug thereof, wherein R⁹ is hydrogen, optionally substituted alkyl, or a nitrogen protecting group.

49. The compound of any preceding embodiment, or a salt, solvate, hydrate, polymorph, co-crystal, tautomer, stereoisomer, isotopically labeled compound, or prodrug thereof, wherein R⁹ is hydrogen.

50. The compound of any preceding embodiment, or a salt, solvate, hydrate, polymorph, co-crystal, tautomer, stereoisomer, isotopically labeled compound, or prodrug thereof, wherein R¹⁰ is —Me.

51. The compound of any preceding embodiment, or a salt, solvate, hydrate, polymorph, co-crystal, tautomer, stereoisomer, isotopically labeled compound, or prodrug thereof, wherein R¹⁰ is hydrogen.

52. The compound of any preceding embodiment, or a salt, solvate, hydrate, polymorph, co-crystal, tautomer, stereoisomer, isotopically labeled compound, or prodrug thereof, wherein at least one instance of R^{11a}, R^{11b}, and R^{11c} is halogen.

53. The compound of any preceding embodiment, or a salt, solvate, hydrate, polymorph, co-crystal, tautomer, stereoisomer, isotopically labeled compound, or prodrug thereof, wherein R¹² is hydrogen, halogen, or —CN.

54. The compound of any preceding embodiment, or a salt, solvate, hydrate, polymorph, co-crystal, tautomer, stereoisomer, isotopically labeled compound, or prodrug thereof, wherein R¹² is hydrogen.

55. The compound of any preceding embodiment, or a salt, solvate, hydrate, polymorph, co-crystal, tautomer, stereoisomer, isotopically labeled compound, or prodrug thereof, wherein R¹² is optionally substituted C₁₋₆ heteroalkyl.

56. The compound of any preceding embodiment, or a salt, solvate, hydrate, polymorph, co-crystal, tautomer, stereoisomer, isotopically labeled compound, or prodrug thereof, wherein R¹² is —CH₂NMe₂.

57. The compound of any preceding embodiment, or a salt, solvate, hydrate, polymorph, co-crystal, tautomer, stereoisomer, isotopically labeled compound, or prodrug thereof, wherein R¹³ is hydrogen.

58. The compound of any preceding embodiment, or a salt, solvate, hydrate, polymorph, co-crystal, tautomer, stereoisomer, isotopically labeled compound, or prodrug thereof, wherein R¹⁴ is hydrogen, halogen, optionally substituted C₁₋₆ alkyl, or optionally substituted C₁₋₆ heteroalkyl.

59. The compound of any preceding embodiment, or a salt, solvate, hydrate, polymorph, co-crystal, tautomer, stereoisomer, isotopically labeled compound, or prodrug thereof, wherein R¹⁴ is hydrogen.

60. The compound of any preceding embodiment, or a salt, solvate, hydrate, polymorph, co-crystal, tautomer, stereoisomer, isotopically labeled compound, or prodrug thereof, wherein R¹³ and R¹⁴ are hydrogen.

61. The compound of any preceding embodiment, or a salt, solvate, hydrate, polymorph, co-crystal, tautomer, stereoisomer, isotopically labeled compound, or prodrug thereof, wherein one of R^{15a}, R^{15b}, and R^{15c} is halogen.

62. The compound of any preceding embodiment, or a salt, solvate, hydrate, polymorph, co-crystal, tautomer, stereoisomer, isotopically labeled compound, or prodrug thereof, wherein n is 0.

63. The compound of any preceding embodiment, or a salt, solvate, hydrate, polymorph, co-crystal, tautomer, stereoisomer, isotopically labeled compound, or prodrug thereof, wherein n is 1.

64. The compound of any preceding embodiment, or a salt, solvate, hydrate, polymorph, co-crystal, tautomer, stereoisomer, isotopically labeled compound, or prodrug thereof, wherein m is 0.

65. The compound of any preceding embodiment, or a salt, solvate, hydrate, polymorph, co-crystal, tautomer, stereoisomer, isotopically labeled compound, or prodrug thereof, wherein m is 1.

66. The compound of any preceding embodiment, or a salt, solvate, hydrate, polymorph, co-crystal, tautomer, stereoisomer, isotopically labeled compound, or prodrug thereof, wherein the compound is any one of the formulae shown in Table 1:

TABLE 1

| | |
|---|---|
| 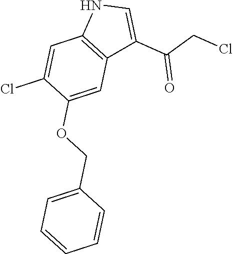 [SMILES: O=C(CCl)c1c[nH]c2cc(Cl)c(OCc3ccccc3)cc12] | 1 |
| 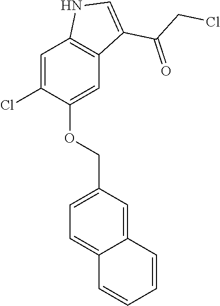 [SMILES: O=C(CCl)c1c[nH]c2cc(Cl)c(OCc3ccc4ccccc4c3)cc12] | 2 |
| 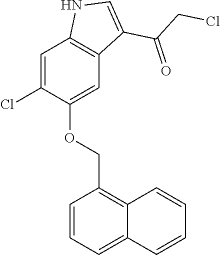 [SMILES: O=C(CCl)c1c[nH]c2cc(Cl)c(OCc3cccc4ccccc34)cc12] | 3 |
| 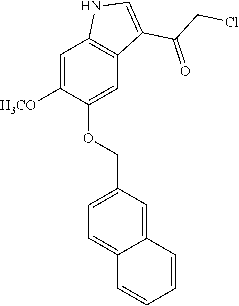 [SMILES: COc1cc2[nH]cc(C(=O)CCl)c2cc1OCc1ccc2ccccc2c1] | 5 |
| 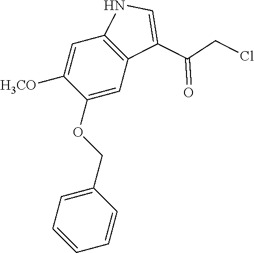 [SMILES: COc1cc2[nH]cc(C(=O)CCl)c2cc1OCc1ccccc1] | 4 |
| 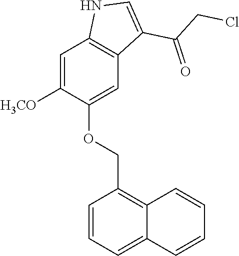 [SMILES: COc1cc2[nH]cc(C(=O)CCl)c2cc1OCc1cccc2ccccc12] | 6 |
| 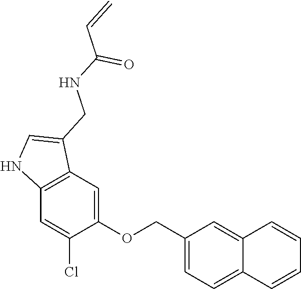 [SMILES: C=CC(=O)NCc1c[nH]c2cc(Cl)c(OCc3ccc4ccccc4c3)cc12] | 9 |
| 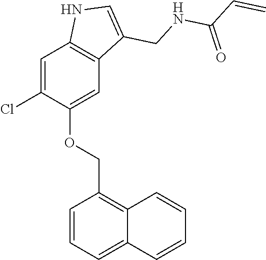 [SMILES: C=CC(=O)NCc1c[nH]c2cc(Cl)c(OCc3cccc4ccccc34)cc12] | 10 |
| 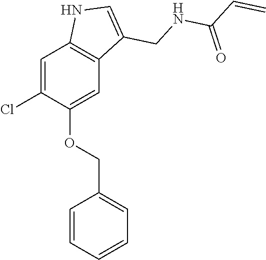 [SMILES: C=CC(=O)NCc1c[nH]c2cc(Cl)c(OCc3ccccc3)cc12] | 11 |
| 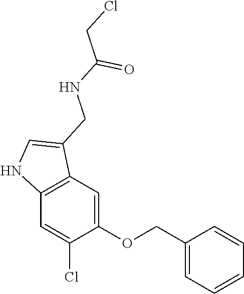 [SMILES: O=C(CCl)NCc1c[nH]c2cc(Cl)c(OCc3ccccc3)cc12] | 12 |
| 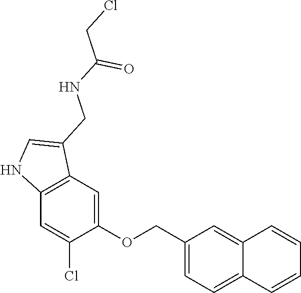 [SMILES: O=C(CCl)NCc1c[nH]c2cc(Cl)c(OCc3ccc4ccccc4c3)cc12] | 13 |
| 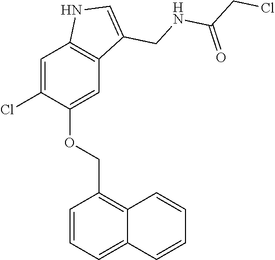 [SMILES: O=C(CCl)NCc1c[nH]c2cc(Cl)c(OCc3cccc4ccccc34)cc12] | 14 |
| 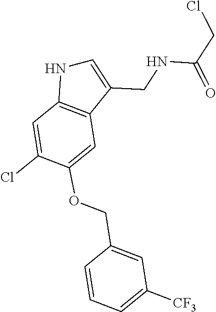 [SMILES: O=C(CCl)NCc1c[nH]c2cc(Cl)c(OCc3cccc(C(F)(F)F)c3)cc12] | 15 |
| 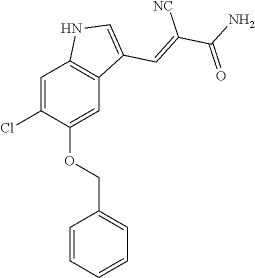 [SMILES: N#C/C(=C\c1c[nH]c2cc(Cl)c(OCc3ccccc3)cc12)C(N)=O] | 16 |
| 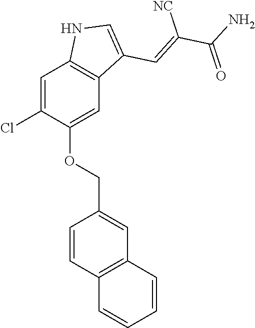 [SMILES: N#C/C(=C\c1c[nH]c2cc(Cl)c(OCc3ccc4ccccc4c3)cc12)C(N)=O] | 17 |
| 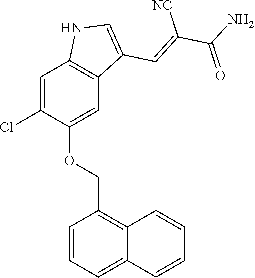 [SMILES: N#C/C(=C\c1c[nH]c2cc(Cl)c(OCc3cccc4ccccc34)cc12)C(N)=O] | 18 |
| 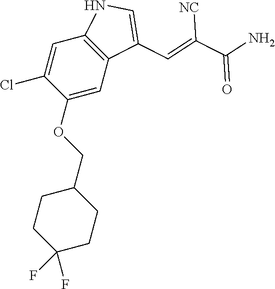 [SMILES: N#C/C(=C\c1c[nH]c2cc(Cl)c(OCC3CCC(F)(F)CC3)cc12)C(N)=O] | 19 |
| 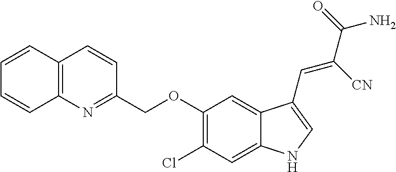 [SMILES: N#C/C(=C\c1c[nH]c2cc(Cl)c(OCc3ccc4ccccc4n3)cc12)C(N)=O] | 20 |
| 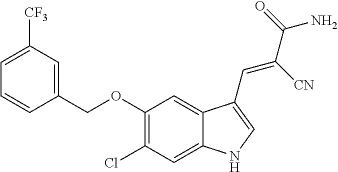 [SMILES: N#C/C(=C\c1c[nH]c2cc(Cl)c(OCc3cccc(C(F)(F)F)c3)cc12)C(N)=O] | 21 |
| 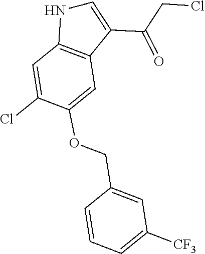 [SMILES: O=C(CCl)c1c[nH]c2cc(Cl)c(OCc3cccc(C(F)(F)F)c3)cc12] | 22 |
| 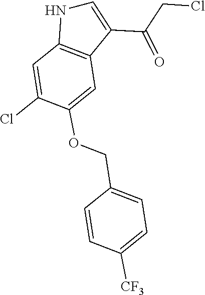 [SMILES: O=C(CCl)c1c[nH]c2cc(Cl)c(OCc3ccc(C(F)(F)F)cc3)cc12] | 23 |
| 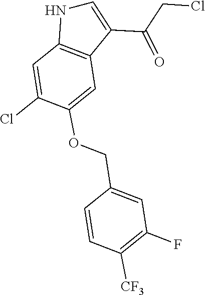 [SMILES: O=C(CCl)c1c[nH]c2cc(Cl)c(OCc3ccc(C(F)(F)F)c(F)c3)cc12] | 24 |
| 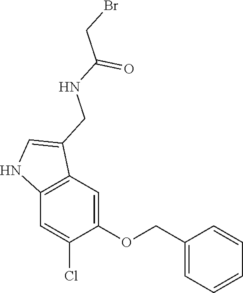 [SMILES: O=C(CBr)NCc1c[nH]c2cc(Cl)c(OCc3ccccc3)cc12] | CV-4-21 |
| 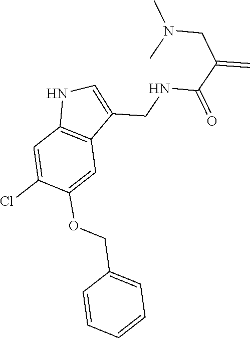 [SMILES: C=C(CN(C)C)C(=O)NCc1c[nH]c2cc(Cl)c(OCc3ccccc3)cc12] | 25 |

67. The compound of any preceding embodiment, or a salt, solvate, hydrate, polymorph, co-crystal, tautomer, stereoisomer, isotopically labeled compound, or prodrug thereof, wherein the compound is not any one of the formulae shown in Table 1.

68. The compound of any preceding embodiment, or a salt, solvate, hydrate, polymorph, co-crystal, tautomer, stereoisomer, isotopically labeled compound, or prodrug thereof, wherein the compound is not of Formula I-A.

69. A compound of formula 7:

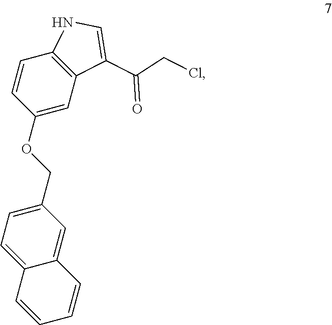 [SMILES: O=C(CCl)c1c[nH]c2ccc(OCc3ccc4ccccc4c3)cc12]

or a salt, solvate, hydrate, polymorph, co-crystal, tautomer, stereoisomer, isotopically labeled compound, or prodrug thereof.

70. A compound of formula 12:

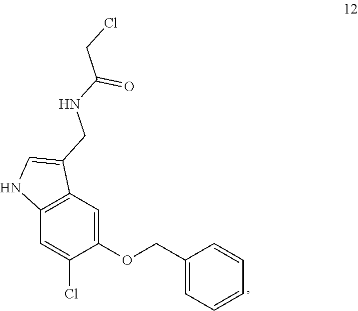 [SMILES: O=C(CCl)NCc1c[nH]c2cc(Cl)c(OCc3ccccc3)cc12]

or a salt, solvate, hydrate, polymorph, co-crystal, tautomer, stereoisomer, isotopically labeled compound, or prodrug thereof.

71. A compound of formula 22:

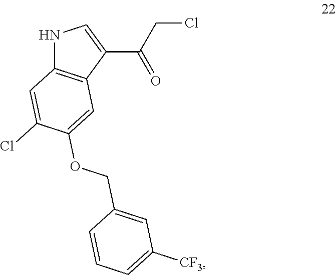 [SMILES: O=C(CCl)c1c[nH]c2cc(Cl)c(OCc3cccc(C(F)(F)F)c3)cc12]

or a salt, solvate, hydrate, polymorph, co-crystal, tautomer, stereoisomer, isotopically labeled compound, or prodrug thereof.

72. A pharmaceutical composition comprising:
the compound of any of embodiments 1-71, or a salt, solvate, hydrate, polymorph, co-crystal, tautomer, stereoisomer, isotopically labeled compound, or prodrug thereof; and
optionally a pharmaceutically acceptable excipient.

73. The pharmaceutical composition of embodiment 72, further comprising an additional pharmaceutical agent.

74. The pharmaceutical composition of embodiment 73, wherein the additional pharmaceutical agent is Sorafenib.

75. The pharmaceutical composition of any of embodiments 72-74, wherein the pharmaceutical composition is formulated for oral administration, intraperitoneal injection, or subcutaneous injection.

76. A kit comprising:
the compound of any of embodiments 1-71, or a salt, solvate, hydrate, polymorph, co-crystal, tautomer, stereoisomer, isotopically labeled compound, or prodrug thereof, or the pharmaceutical composition of any of embodiments 72-75; and
instructions for using the compound, or salt, solvate, hydrate, polymorph, co-crystal, tautomer, stereoisomer, isotopically labeled compound, or prodrug thereof, or pharmaceutical composition.

77. A method of treating a disease in a subject in need thereof, the method comprising administering to the subject an effective amount of:
the compound of any of embodiments 1-71, or salt, solvate, hydrate, polymorph, co-crystal, tautomer, stereoisomer, isotopically labeled compound, or prodrug thereof; or
the pharmaceutical composition of any one of embodiments 72-75.

78. A method of preventing a disease in a subject in need thereof, the method comprising administering to the subject an effective amount of:
the compound of any one of embodiments 1-71, or salt, solvate, hydrate, polymorph, co-crystal, tautomer, stereoisomer, isotopically labeled compound, or prodrug thereof; or
the pharmaceutical composition of any one of embodiments 72-75.

79. The method of embodiment 77 or 78, wherein the effective amount is effective in inhibiting TEAD transcription activity.

80. The method of any of embodiments 77-79, wherein the effective amount is about 1 mg/kg to about 200 mg/kg.

81. The method of any of embodiments 77-80, wherein the effective amount is about 10 mg/kg to about 100 mg/kg.

82. The method of any of embodiments 77-81, wherein the effective amount is about 20 mg/kg to about 55 mg/kg.

83. The method of any of embodiments 77-82, wherein the effective amount is about 25 mg/kg.

84. The method of any of embodiments 77-83, wherein the effective amount is about 50 mg/kg.

85. The method of any of embodiments 77-84, wherein the disease is cancer.

86. The method of any of embodiments 77-85, wherein the cancer is breast, pancreatic, liver, colorectal, lung, ovarian, or prostate cancer.

87. The method of any of embodiments 77-86, wherein the cancer is hepatocellular carcinoma, triple negative breast cancer, mesothelioma, or hepatoblastoma.

88. The method of any of embodiments 77-87, wherein the method further comprises co-administering an additional pharmaceutical agent.

89. The method of embodiment 88, wherein the additional pharmaceutical agent is co-administered on the same schedule as the compound, or salt, solvate, hydrate, polymorph, co-crystal, tautomer, stereoisomer, isotopically labeled compound, or prodrug thereof; or to the pharmaceutical composition.

90. The method of embodiment 88 or 89, wherein the co-administration increases sensitivity to the additional pharmaceutical agent.

91. The method of any of embodiments 88-90, wherein the co-administration increases sensitivity to the compound, or salt, solvate, hydrate, polymorph, co-crystal, tautomer, stereoisomer, isotopically labeled compound, or prodrug thereof; or to the pharmaceutical composition.

92. The method of any of embodiments 88-91, wherein the additional pharmaceutical agent is Sorafenib.

93. The method of any of embodiments 88-92, wherein the additional pharmaceutical agent is administered in the amount of about 1 mg/kg to about 200 mg/kg.

94. The method of any of embodiments 88-93, wherein the additional pharmaceutical agent is administered in the amount of about 50 mg/kg to about 150 mg/kg.

95. The method of any of embodiments 88-94, wherein the additional pharmaceutical agent is administered in the amount of about 100 mg/kg.

96. A method of inhibiting the activity of TEAD in a subject in need thereof, the method comprising administering to the subject an effective amount of:
the compound of any of embodiments 1-71, or salt, solvate, hydrate, polymorph, co-crystal, tautomer, stereoisomer, isotopically labeled compound, or prodrug thereof; or
the pharmaceutical composition of any of embodiments 72-75.

97. The method of any of embodiments 77-96, wherein the subject is a human.

98. The method of any of embodiments 77-97, wherein the subject is a human aged 18 and older.

99. A method of inhibiting the activity of TEAD in a cell, tissue, or biological sample the method comprising contacting the cell, tissue, or biological sample with an effective amount of:
the compound of any of embodiments 1-71, or salt, solvate, hydrate, polymorph, co-crystal, tautomer, stereoisomer, isotopically labeled compound, or prodrug thereof; or
the pharmaceutical composition of any of embodiments 72-75.

100. The method of embodiment 99, wherein the cell, tissue, or biological sample is in vitro.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### INTRODUCTION

The Hippo signaling pathway exerts a critical role in organ size control and tumorigenesis through the regulation of cell proliferation, cell survival, and cell migration.¹⁻³ In addition, more evidence suggests that the Hippo pathway has a significant impact on patient prognosis and the regulation of chemotherapeutic drug resistance.⁴ The core Hippo pathway, which is a kinase cascade, has been well established in both Drosophila and mammals. Activation of the Hippo pathway phosphorylates and activates its core kinase cascade including MST½ and LATS½ kinases that phosphorylate the downstream transcription co-activators yes-associated protein (YAP) and its paralog transcriptional co-activator with PDZ-binding motif (TAZ) leading to their cytoplasmic retention through 14-3-3 protein interactions or ubiquitin-mediated proteasome degradation.⁵⁻⁷ Inactivation of the Hippo kinase core leads to YAP/TAZ dephosphorylation and translocation into the nucleus, where they interact predominantly with TEA/TEF domain transcription factors (TEAD1-4).^{8,9} This consequently initiates the transcription and translation of downstream genes involved in cell proliferation and survival, such as connective tissue growth factor (CTGF), and cysteine-rich angiogenic inducer 61 (CYR61).¹⁰⁻¹³ The deregulation of the Hippo pathway has been reported at a high frequency in a broad range of different human carcinomas, including liver, colorectal, lung, ovarian, and prostate cancers and it often correlates with poor patient prognosis.¹⁴⁻¹⁸ Therefore, targeting the YAP/TEAD complex activity, which is essential in regulating the transcriptional outcome of Hippo pathway, has great potential for the treatment of cancers and diseases with altered Hippo signaling.¹⁹

Liver cancer is a global concern due to the elevated prevalence of risk of factors such as obesity, diabetes, and other metabolic syndromes. Hepatocellular carcinoma (HCC) is one of the most common malignant cancers, and is the most common malignant liver cancer, with increasing incidence and mortality in recent years in the United States.²⁰ Disease progression in patients involves severe liver inflammation and fibrosis that are correlated with deregulation of various signaling pathways and genetic aberrations.^{21,22} Patients with advanced HCC have very poor prognoses.²³ HCC remains a deadly disease with unmet medical needs. Only 12% have a 1-year survival with current treatments, highlighting the urgent need to develop new and more effective therapeutic approaches for HCC.²⁸ The United States Food and Drug Administration (US-FDA, Silver Spring, USA) approved systemic therapies include, atezolizumab-bevacizumab combination, sorafenib, and lenvatinib in the first line setting.²⁴⁻²⁶ These current treatment options, however, are far from being satisfactory due to rapid emergence of drug resistance and unwanted off-target toxicities.²⁷ Drug resistance to sorafenib involves pathways such as the altered Hippo pathway. Sorafenib enhances yes-associated protein (YAP) nuclear accumulation and activation, thereby promoting sorafenib resistance through inhibiting apoptosis in HCC cells.²⁹⁻³¹ The above data thus suggest that YAP is a relevant target for the treatment of advanced HCC.

Hepatoblastoma (HB) is a rare malignant tumor of the liver in pediatric populations. It is found in 90% of cases before 3 years of age.⁶⁷ YAP1S127A withdrawal significantly regresses hepatoblastoma, implicating YAP1 as a therapeutic target for HB.⁶⁸ Novel T-cell therapy ET140203 (ARTEMIS) has been granted orphan drug designation by the FDA for the treatment of pediatric HB.⁶⁹ No small-molecule drug is available for HB. Preoperative or postoperative chemotherapy is still the major way for the treatment. So, HB remains a deadly disease with unmet medical needs.

YAP is partially disordered with no known catalytic activity, rendering it an intractable drug target.³² However, the TEAD proteins (e.g., TEAD1, TEAD2, TEAD3, and TEAD4) are proposed to be a therapeutic target for the intervention of the Hippo pathway.³³ Since the release of crystal structures of TEADs, efforts have been put into targeting the YAP-TEAD complex.³⁴⁻³⁶ In general, there are three strategies targeting TEAD/YAP complex, including targeting YAP, focusing on the YAP binding interface of TEAD, and inhibiting TEAD palmitoylation.

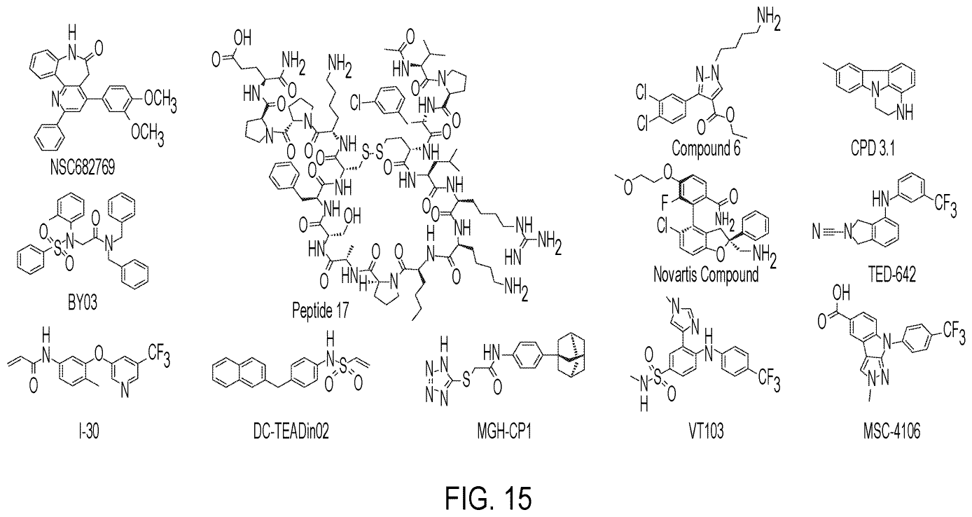

Functionally, the most attractive therapeutic targets in the Hippo pathway are the oncoproteins YAP and TAZ as the final common conduits of the Hippo pathway. However, YAP is a disordered protein, making drug discovery more challenging. Verteporfin (VP), which is FDA approved, was first discovered as a YAP-TEAD inhibitor in a luciferase reporter assay screening by Liu-Chittenden et al. in 2012.37 An appreciable VP fluorescence coeluted with the YAP protein peak but not with the TEAD2 protein peak, demonstrating that VP selectively binds YAP.³⁷ However, recent work indicates that verteporfin may work through other mechanisms, including inhibition of p62.³⁸ Through a similar luciferase reporter assay screening, T. Saunders et al. identified compound NSC682769, which appears to directly bind to YAP and inhibit its association with all four TEAD family members (FIG. 15).39 Moreover, NSC682769 attenuates YAP expression by binding to YAP and resulting in targeted degradation of the protein.

The second strategy involves direct inhibition of the YAP-TEAD complex formation at either YAP interface 3 (Q-loop) or YAP interface 2 (α-helix) binding interface because of the importance of molecular interactions between YAP and TEAD at these areas.⁴⁰⁻⁴² This protein-protein interaction (PPI) inhibition might pose a challenge due to the large interaction areas involved, but inhibition has been accomplished by inhibitory peptides and small molecules. In 2014, Zhang et al. developed the potent cyclic peptide inhibitor Peptide 17 (FIG. 15) by applying conformational constraints to a mutated Q-loop region (YAP84-100, D94A) via a disulfide bridge (FIG. 15).43 However, peptide molecules are usually not orally bioavailable and need to be delivered via injection, rendering them less desirable from a drug standpoint.⁴⁴ Besides peptide inhibitors, small molecules are also believed to be functional in YAP-TEAD disruption and are therapeutically promising. Only a few small molecules are reported to disrupt YAP-TEAD binding by binding to the TEAD surface. CPD 3.1 was identified from a docking screening that virtually screened 8 million compounds to identify those that occupy the TEAD interface 3 pocket.⁴⁵ CPD3.1 has been shown to disrupt YAP/TEAD interaction in co-immunoprecipitation (co-IP) assays and inhibits TEAD reporter activity. However, there is no direct proof of TEAD binding via structural studies. Interestingly, Compound 6 and BY03, which inhibit YAP/TEAD by targeting interface 2, were reported by two groups nearly simultaneously, however both showed weak activities in vitro (FIG. 15).^{46,47} While the binding site of Compound 6 was confirmed by an X-ray crystal structure (PDB code 6S6J), there are only docking studies to suggest the binding mode for BY03. Novartis reported the first class of small molecules that potently disrupt YAP/TEAD interaction by directly occupy interface 3, with the binding mode demonstrated by X-ray crystal structure (FIG. 15).⁴⁸ Exploiting crystal structure information, they identified the first representatives of a class of YAP/TEAD protein-protein interaction inhibitors, an unprecedented discovery. Importantly, some of the compounds reached a single digit nanomolar inhibition of YAP/TEAD-dependent reporter gene expression in the NCI-H2052 cells.

The third strategy focuses on the TEAD lipid binding pocket. Recent studies discovered that human TEADs require auto-lipidation (palmitoylation or myristoylation) at conserved cysteine residues (for example, C380 on TEAD2) under physiological conditions to become functional.^{49,50} This post-translational modification on TEADs increases stability and activity of TEAD transcription factors by regulating their interaction with YAP/TAZ.

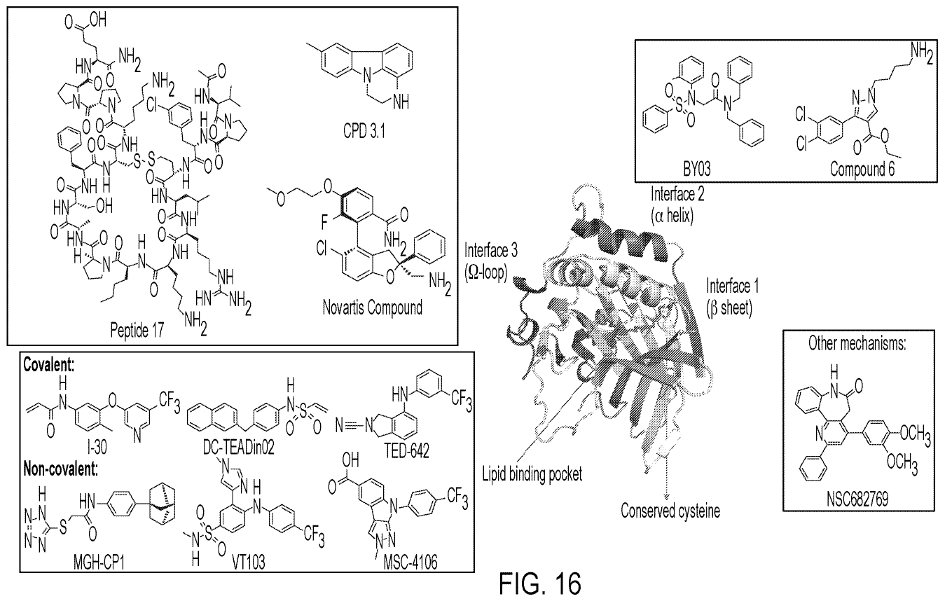

This deep and hydrophobic TEAD lipid binding pocket may be accessed by small molecules (a “druggable pocket”). Therefore, the inhibition of TEAD palmitoylation is proposed as a further alternative strategy to inhibit YAP/TEAD activity. So far, several reversible and irreversible TEAD palmitoylation modulators that bind to the lipid-binding pocket have been explored and reported. TED-642, 1-30 and DC-TEADin02 are covalent autopalmitoylation inhibitors and TEAD stabilizers that downregulate TEAD transcriptional activity (FIG. 15).³⁶′ ^{51,52} However, MGH-CP1, VT103 and MSC-4106 inhibit TEAD autopalmitoylation through a reversible mechanism (FIG. 15).⁵³⁻⁵⁵ Notably, certain research activities have already been successful and provided three clinical development compounds, but the chemical structures of these are not yet known (VT-3989: NCT04665206, IAG-933: NCT04857372, and IK-930: NCT05228015).³⁶ A proteolysis targeting chimera (PROTAC) strategy was also used, aiming to degrade TEAD.^{70,71} For example, based on their reported YAP/TEAD protein-protein interaction inhibitors, Novartis developed several potent TEAD PROTACs with single digit nanomolar DC50 values very recently.⁷¹ The crystal structure of the YAP/TEAD1 complex shows interface 1, interface 2, and interface 3 (FIG. 16). The compounds discussed earlier are categorized based on their interactions with specific binding sites.

Provided herein are results of a high-throughput docking campaign that led to the identification of a moderate TEADs inhibitor C-3. Structure-based design and Structure-Activity Relationship (SAR) analysis revealed new covalent compounds, including compound 22, with greatly increased potency. Compound 22 exhibited potent inhibition of YAP/TEAD-based transcription, leading to downregulated CTGF and CYR61 expression, as well as suppression of proliferation in human HCC and HB cell lines in cell culture.

Additionally, compound 22 successfully stopped the growth of human Huh7 tumors in subcutaneously xenografted mice. The effects were accompanied by lower toxicity to normal liver cells. Furthermore, its efficacy extended to effectively impeding murine HB development in vivo. This study demonstrates that inhibition of YAP/TEAD transcription by compounds provided herein has a therapeutic potential for the treatment of HCC and HB.

### Results and Discussion

### Docking-Based Virtual Screening and Hit Validation.

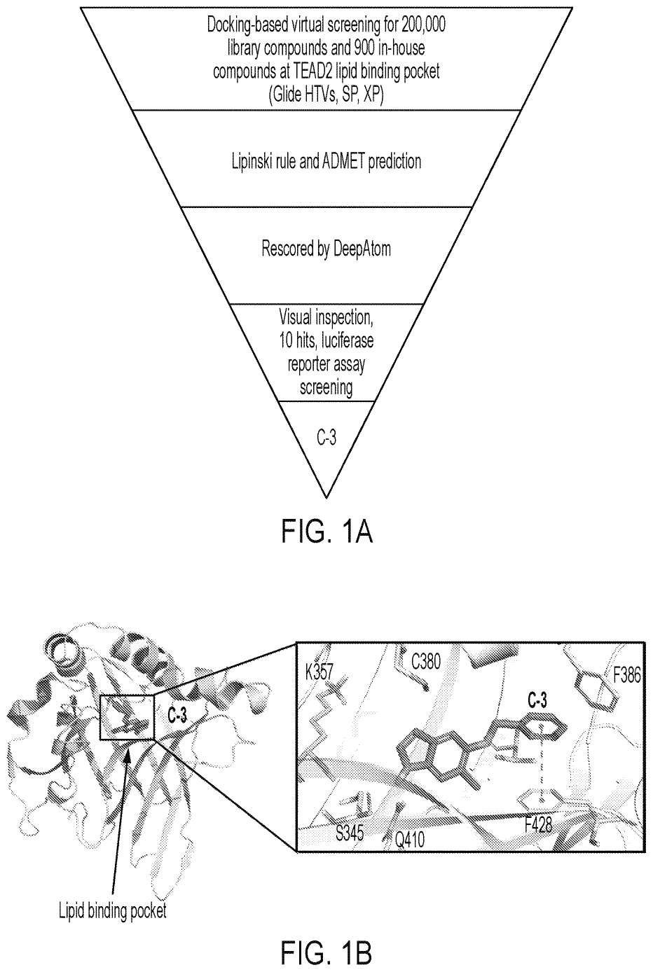

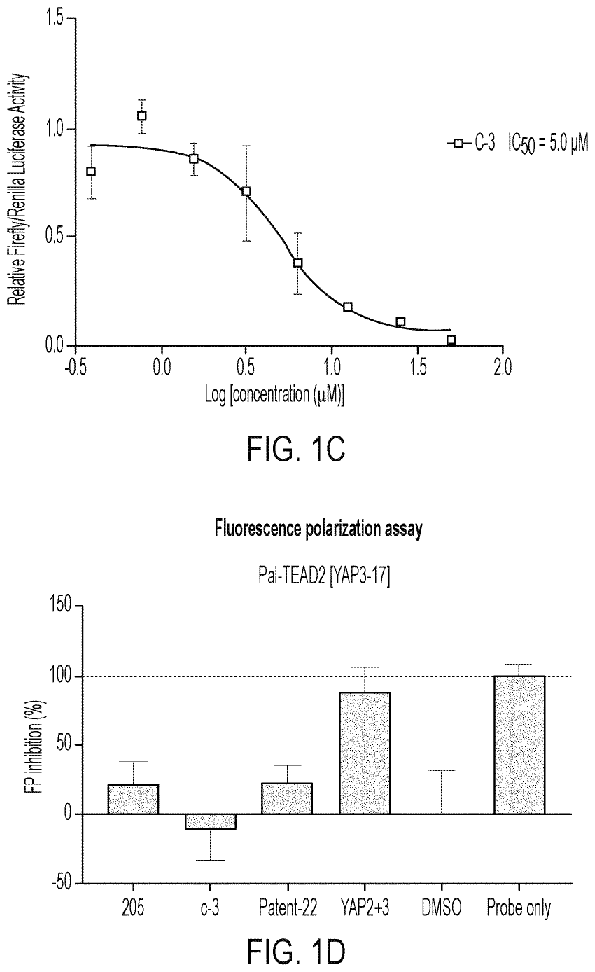

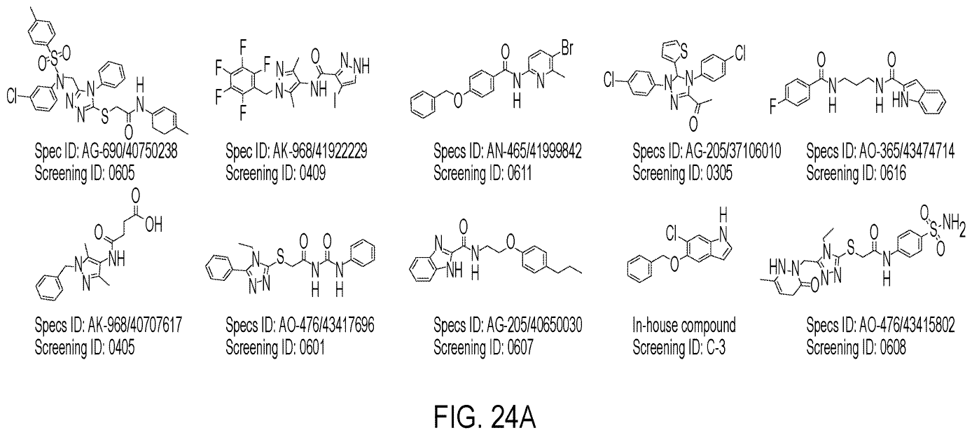

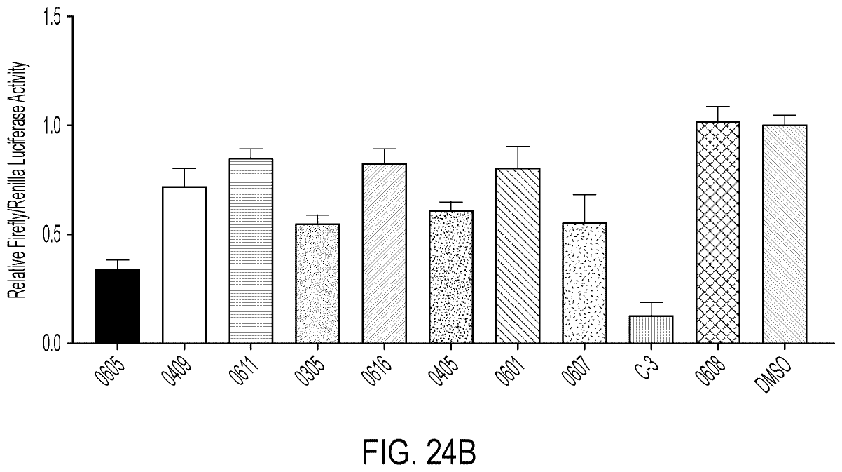

In the present study, a docking-based virtual screening strategy was used to identify promising hits targeting the TEAD2 lipid binding pocket (FIG. 1A). Higher mRNA expression of TEAD2 serves as a prognostic indicator for poor outcomes in HCC patients. The lipid-binding pocket of TEAD2 was selected for docking experiments in pursuit of liver cancer therapies. A combination of 200,000 commercially available compounds from the Specs database and 900 in-house compounds were utilized for virtual screening. Standard Precision mode in Glide panel was used for docking.⁷²⁻⁷⁴ Compounds were filtered by the Lipinski rule and ADMET predictions.⁷⁵ The compounds that showed lower predicted toxicity and better pharmacokinetic profiles were preferred. The remaining 100 compounds were rescored with an in-house DeepAtom protein-ligand binding deep-learning model.⁵⁶ Visual inspection of the binding modes focused on the hydrophobic pocket and the opening of the pocket, where a conserved cysteine residue is located, and ten candidates were chosen for biological validation (FIG. 24A).⁵⁷ A TEAD-specific luciferase reporter assay was used to examine the transcription inhibition activities of those ten compounds (FIG. 24B).³⁷ Compound C-3, which displayed a good binding mode within the TEAD2 lipid binding pocket, showed moderate inhibition activity in the luciferase reporter assay with an IC₅₀ value of 5.0 μM (FIGS. 1B-1C).

### Design and SAR Analysis of Covalent Inhibitors.

Despite the initial skepticism of pharmaceutical industry to develop covalent inhibitors, in the last 50 years, the development of covalent inhibitors has generated considerable interest.^{58,59} Numerous drug candidates are progressing through clinical trials or have been approved by the FDA.⁶⁰ Notable examples include BNP7787 and afatinib.⁶⁰

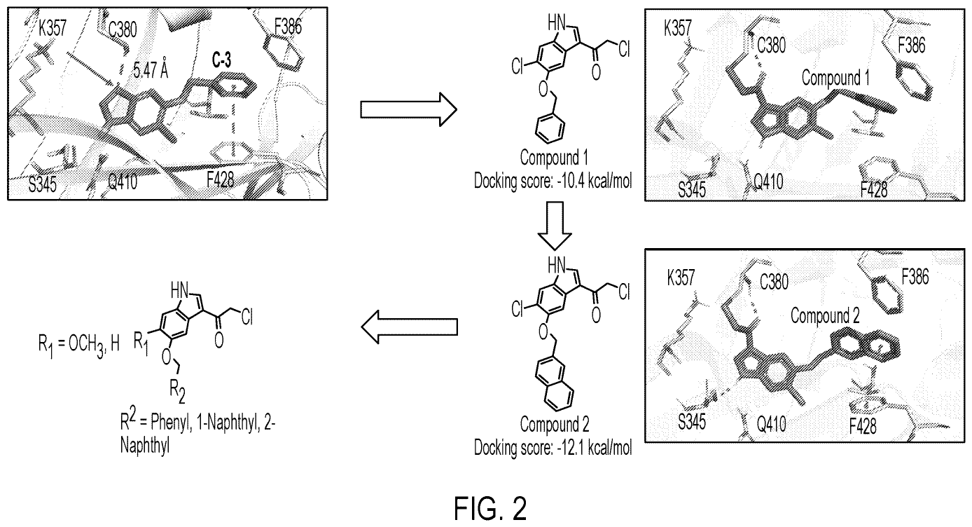

To further develop more potent small molecular TEAD inhibitors, covalent inhibitors were rationally designed, which depend on the nucleophilic cysteine adjacent to the binding pocket, which is 5.47 A away from the indole 3 position carbon of C-3 (FIG. 2). A series of compounds featuring a chloromethyl ketone moiety were synthesized (Schemes 1-4). The inhibitory rate of this series of compounds was tested using 8×GTIIC-luciferase reporter assay at 10 μM. The mRNA expression of TEAD2 and VGLL4 was significantly higher in HCC compared with the normal control samples, and the mRNA expression of TEAD2 was higher in advanced stages than in early stages.^{30,61} The compounds were evaluated for cytotoxicity and using in vitro cell proliferation assay against liver cancer cell line Huh7 (Table 2).

TABLE 2
Anti-proliferative Activities of Compounds 1-6.

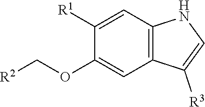

| | | | | Luciferase | | |
|---|---|---|---|---|---|---|
| | | | | reporter | Cell | |
| | | | | assay | proliferation | |
| | | | | inhibitory | assays IC₅₀, | Inhibitory |
| | | | | rate (10 | (μM) ^{a} | rate ^{b} |
| Compound | R¹ | R² | R³ | μM) | Huh7 | AML12 |
| 1 | Cl | 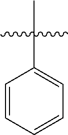 [SMILES: *c1ccccc1] | —COCH₂Cl | 74.6% | 2.7 ± 0.2 | 36.2% |
| 2 | CI | 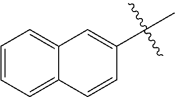 [SMILES: *c1ccc2ccccc2c1] | —COCH₂Cl | 95.6% | 2.3 ± 0.6 | 32.1% |
| 3 | Cl | 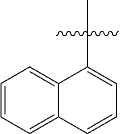 [SMILES: *c1cccc2ccccc12] | —COCH₂Cl | 69.4% | 1.6 ± 0.3 | 50.3% |
| 4 | OCH₃ | 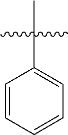 [SMILES: *c1ccccc1] | —COCH₂Cl | 53.7% | 3.8 ± 0.4 | 46.1% |
| 5 | OCH₃ | 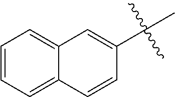 [SMILES: *c1ccc2ccccc2c1] | —COCH₂Cl | 93.9% | 2.4 ± 0.5 | 77.4% |
| 6 | OCH₃ | 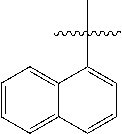 [SMILES: *c1cccc2ccccc12] | —COCH₂Cl | 64.5% | 2.5 ± 0.8 | 62.1% |
| 7 | H | 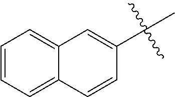 [SMILES: *c1ccc2ccccc2c1] | —COCH₂Cl | 5.3% | >20 | ND |
| 8 | H | 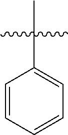 [SMILES: *c1ccccc1] | —COCH₂Cl | 20.5% | 10.5 ± 0.9 | ND |
| C-3 | Cl | 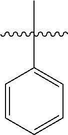 [SMILES: *c1ccccc1] | H | 68.3% | 32.5 | 20% |

^{a} Cells were treated with compounds for 3 days, and cell growth was determined by a CCK8 assay; values reported are the mean ± SD of three independent experiments
^{b} The inhibitory rate means the inhibitory percentage of AML12 cell growth when treated with 10 μM compounds for 3 days.

Notably, compound 1, with the addition of the covalent moiety, showed significant improvement in TEAD inhibition activity and on Huh7 cell growth inhibition activity compared to C-3. Considering the hydrophobicity of the pocket, the phenyl group was replaced with more hydrophobic naphthalene moiety to provide compound 2, which showed better cellular inhibitory activity than compound 1. Based on compound 2, further optimization was conducted, both with regard to substitution on the indole 6 position (R¹) and with regard to the ether moieties (R²). First, for R¹, the chlorine was changed to a methoxy group or a hydrogen. As methoxy is a little bit larger than chlorine, it may allow for increased interaction within the hydrophobic binding pocket. Introduction of methoxy decreased the CLogP of the compound, improving compound solubility. R¹ was changed to hydrogen to assess the impact of substituents at this position. Second, for R², substitution of the naphythyl group was changed to the 1 position and the 2 position to explore the SAR and geometry of this arene.

From the results of the luciferase reporter assay and cell proliferation assay, a preliminary SAR was determined (Table 2). In terms of R¹ substitution, Cl was more favorable than OCH₃ and H. Introducing a methoxy substitution resulted in a moderate loss of activity. However, changing R¹ to hydrogen decreased the cellular activity by more than five-fold. The hydrophobic R² substituents were shown to play an important role. The compounds bearing a naphthalene moiety showed better Huh7 cell growth inhibition activity than phenyl compounds. The substitution position of naphthalene was tolerated as it had a minimal impact on activity. As a comparator, the normal liver cell line AML12 was used to test the toxicity of those compounds to healthy cells, and all compounds inhibited AML12 cell growth at 10 M. compound 2 was the least toxic of the covalent compounds, as compound 2 demonstrated a 32% inhibitory rate of AML cells at 10 M.

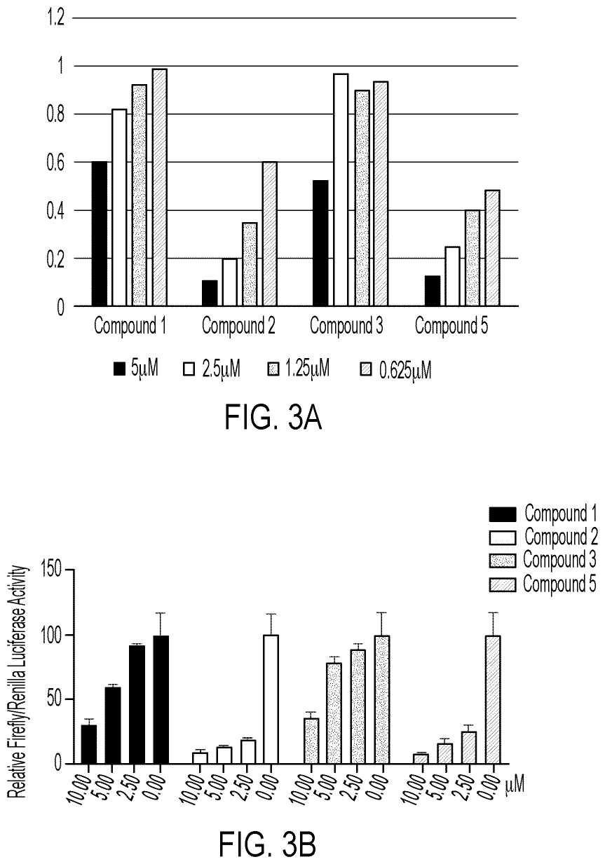

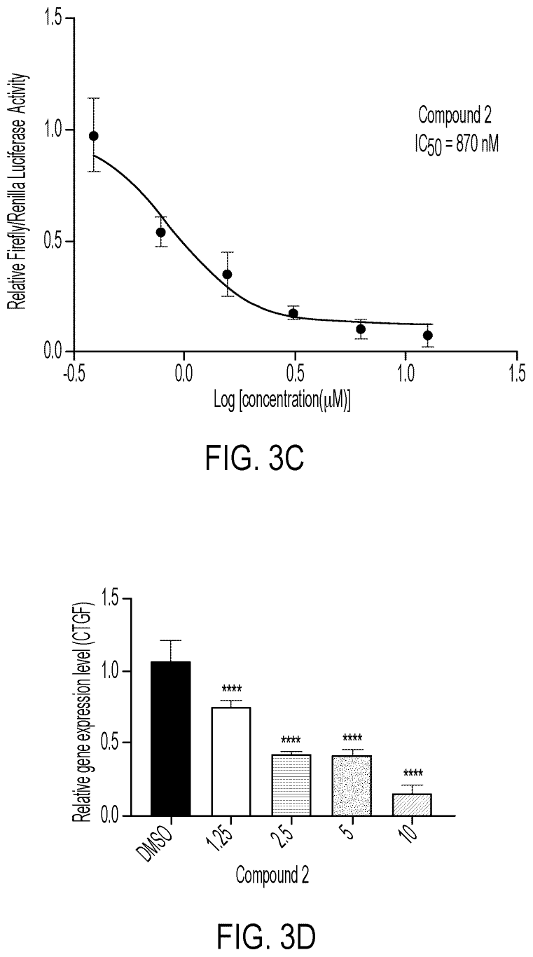

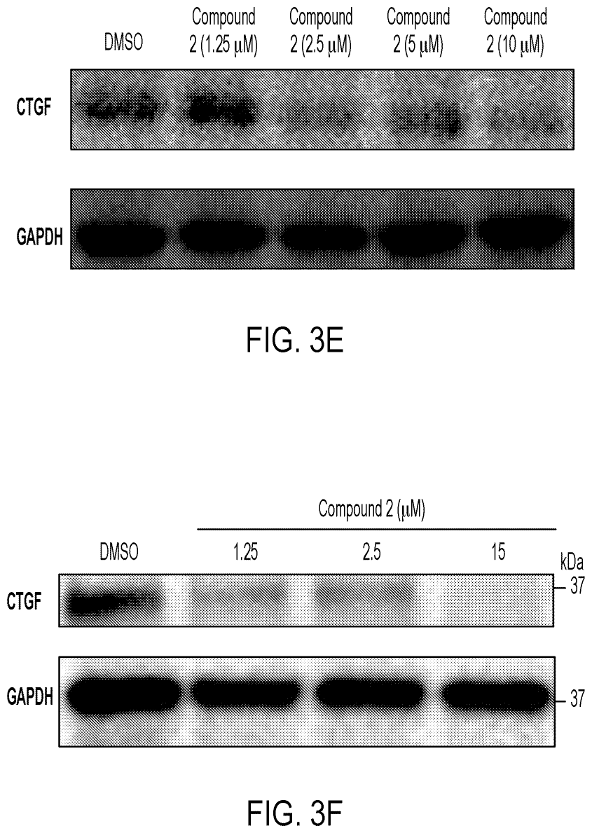

Next, the inhibition of TEAD transcription activity of compounds was tested varying the dosage using the 8×GTIIC-luciferase reporter assay. Compounds 2 and 5 both showed potent inhibition activity (FIGS. 3A-3B). 2 has the IC₅₀ value of 870 nM (FIG. 3C). As 5 showed high toxicity towards normal liver cell line AML12, 2 was selected for further investigation. The aim was to determine whether its inhibition of TEAD transcription can lead to downregulation of the YAP-TEAD target genes. CTGF is one of the best characterized (bona fide) target gene of YAP and TAZ. CTGF contributes to HCC cell dedifferentiation, expression of inflammation-related genes involved in carcinogenesis, and in vivo HCC cell growth.⁶³ The results showed that the expression levels for CTGF mRNA were significantly decreased by 2 in a dose-dependent manner in the Huh7 HCC cell line (FIG. 3D). The corresponding CTGF protein expression levels were examined by western blotting analysis, and a similar dose-dependent decrease in CTGF protein expression was observed (FIGS. 3E-3F).

The initial SAR of these eight compounds demonstrated that the indole core provided a new class of potent TEAD inhibitors not previously reported in the literature. With this new scaffold in hand, efforts focused on designing more potent compounds with reduced toxicity. Without wishing to be bound by any particular theory, the toxicity of the first eight compounds may result from the high reactivity of the covalent warhead. So, the more reactive chloromethyl ketone was changed to lower reactive covalent warheads such as acrylamide, α-chloroacetamide and α-cyanoacrylamide.⁶⁴ The acrylamide and α-chloroacetamide are less reactive than the α-chloroketone due to an extended electron cloud over the conjugated alkenyl and carbonyl moieties and are commonly used covalent warheads.⁶⁴ The concerns of irreversibility, non-specific hyperreactivity, and idiosyncratic toxicities (IDTs) have lately catalyzed the development of long-lived reversible covalent inhibitors.⁶⁵ Therefore, the cyanoacrylamide warhead was used, as it may impart long-lived reversible covalent interaction.

In addition, without wishing to be bound by any particular theory, the CLogP of 2 may be too high, around 5. A CLogP of 4 or below is considered as a cutoff for drug-like properties.⁷⁶ And also, without wishing to be bound by any particular theory, the naphthalene may be too flat and hydrophobic, which may increase the possibility of off-target toxicity.

These factors could be addressed by lowering the lipophilicity and/or reducing the number of aromatics.⁶⁶ Naphthalene was changed to cyclohexane, 4,4-difluorocyclohexyl, 2-quinolinyl, m-trifluoromethylphenyl, p-trifluoromethylphenyl, and m-fluoro-p-trifluoromethylphenyl groups. The inventors hypothesized that sp3 carbons of the cyclohexyl groups may improve solubility and provide improved specificity. A more polar group, such as quinoline, was also used to improve solubility and further decrease the CLogP of the compound. The various trifluomethylphenyl groups, used in some reported TEAD inhibitors,^{54,55} were also explored in the scaffold.

As the series of compounds had limited solubility, compound 25 was synthesized to improve the solubility by installation of soluble groups on the covalent warhead. 8×GTIIC-luciferase reporter assay and Huh7 cell proliferation assay of those compounds were conducted. The most commonly used acrylamide warhead reduced the cell growth inhibition activities., The α-chloroacetamide derivatives, however, displayed slightly improved cellular activities compared to the chloroketone derivatives. Among them, compound 14 showed the most potent Huh7 cell growth inhibition activity with an IC₅₀ of 0.8 M, but significant cytotoxicity for AML12 was also observed. However, most of the α-cyanoacrylamide derivates showed high toxicities or low Huh7 cell growth inhibition. Installation of soluble groups on the covalent warhead resulted in decreased cellular activity. In terms of the hydrophobic R² portion, 4,4-difluorocyclohexane and quinoline both reduced in vitro activities. However, 3-trifluomethylphenyl showed improved activity compared to phenyl and naphthalene. The position of the substituent impacted activity; for example, the 3-CF₃ derivative compound 22 was 16-fold more active than the para-analogue compound 23. Compound 22 obtained by replacing the phenyl group of compound 1 with the 3-trifluomethylphenyl showed potent growth inhibition of Huh7 cells with an IC₅₀ value of 0.9 M. Moreover, compound 22 was least toxic to AML12 cells at 10 μM compared to all other synthesized covalent compounds.

In addition, the substitution position of CF₃ was changed, and a F group was introduced at the meta position to get compounds 23 and 24, respectively.

TABLE 3
Cellular Activities of Compounds 9-24.

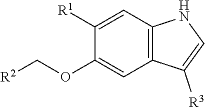

| | | | | Luciferase | | |
|---|---|---|---|---|---|---|
| | | | | reporter | Cell | |
| | | | | assay | proliferation | |
| | | | | inhibitory | assay IC50, | Inhibitory |
| | | | | rate (10 | μM ^{a} | rate ^{b} |
| Cpd | R¹ | R² | R³ | μM) | Huh7 | AML12 |
| 9 | Cl | 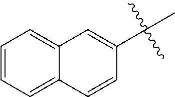 [SMILES: *c1ccc2ccccc2c1] | 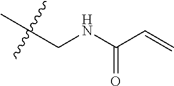 [SMILES: *CNC(=O)C=C] | 1.5% | 30.2 ± 2.5 | 0% |
| 10 | Cl | 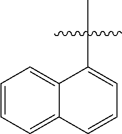 [SMILES: *c1cccc2ccccc12] | | 10.6% | 14.4 ± 1.2 | 0% |
| 11 | Cl | 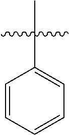 [SMILES: *c1ccccc1] | | ND | >50 | 0% |
| 12 | Cl | 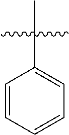 [SMILES: *c1ccccc1] | 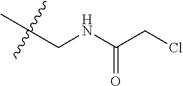 [SMILES: *CNC(=O)CCl] | 97.8% | 1.2 ± 0.3 | 34.2% |
| 13 | Cl | 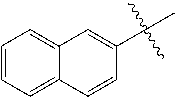 [SMILES: *c1ccc2ccccc2c1] | | 67.4% | 5.7 ± 1.3 | 43.4% |
| 14 | Cl | 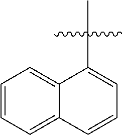 [SMILES: *c1cccc2ccccc12] | | 81.5% | 0.8 ± 0.2 | 54.1% |
| 15 | Cl | 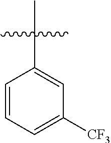 [SMILES: *c1cccc(C(F)(F)F)c1] | | 47.9% | 8.3 ± 0.8 | 41.1% |
| 16 | Cl | 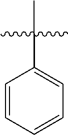 [SMILES: *c1ccccc1] | 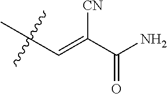 [SMILES: */C=C(\C#N)C(N)=O] | ND | >50 | 0% |
| 17 | Cl | 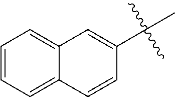 [SMILES: *c1ccc2ccccc2c1] | | 47.3% | 4.1 ± 0.6 | 60.2% |
| 18 | Cl | 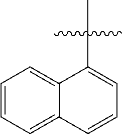 [SMILES: *c1cccc2ccccc12] | | 51.3% | 70% (10 μM) | 70.3% |
| 19 | Cl | 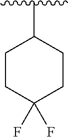 [SMILES: *C1CCC(F)(F)CC1] | | 60.1% | 5.3 ± 0.5 | 30.1% |
| 20 | Cl | 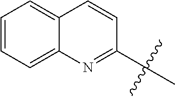 [SMILES: *c1ccc2ccccc2n1] | | ND | 68.7 ± 5.4 | 0% |
| 21 | Cl | 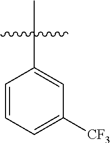 [SMILES: *c1cccc(C(F)(F)F)c1] | | 59.7% | 5.2 ± 0. 6 | 97.1% |
| 22 | Cl | 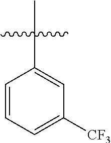 [SMILES: *c1cccc(C(F)(F)F)c1] | | 93.6% | 0.9 ± 0.3 | 22.1% |
| 23 | Cl | 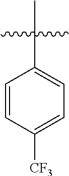 [SMILES: *c1ccc(C(F)(F)F)cc1] | —COCH₂Cl | ND | 16.9 | ND |
| 24 | Cl | 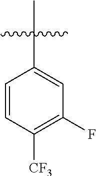 [SMILES: *c1ccc(C(F)(F)F)c(F)c1] | | ND | 1.8 | ND |
| CV-4-21 | Cl | 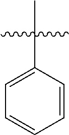 [SMILES: *c1ccccc1] | 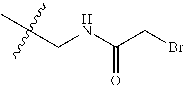 [SMILES: *CNC(=O)CBr] | ND | ND | ND |
| 25 | Cl | 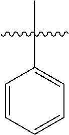 [SMILES: *c1ccccc1] | 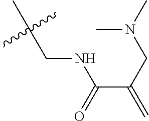 [SMILES: *CNC(=O)C(=C)CN(C)C] | 71.8% | 2.0 ± 0.7 | 37.2% |

^{a} Cells were treated with compounds for 3 days, and cell growth was determined by a CCK8 assay; values reported are the mean ± SD of three independent experiments
^{b} The inhibitory rate means the inhibitory percentage of AML12 cell growth when treated with 10 μM compounds for 3 days.

### Cellular Activity of Compounds 22 and 12.

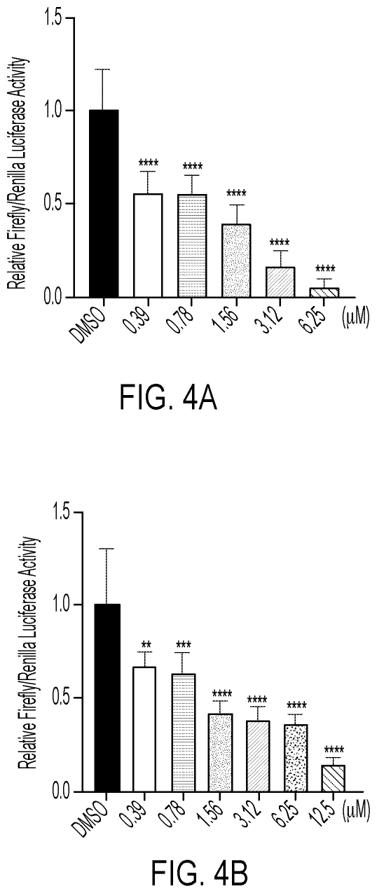

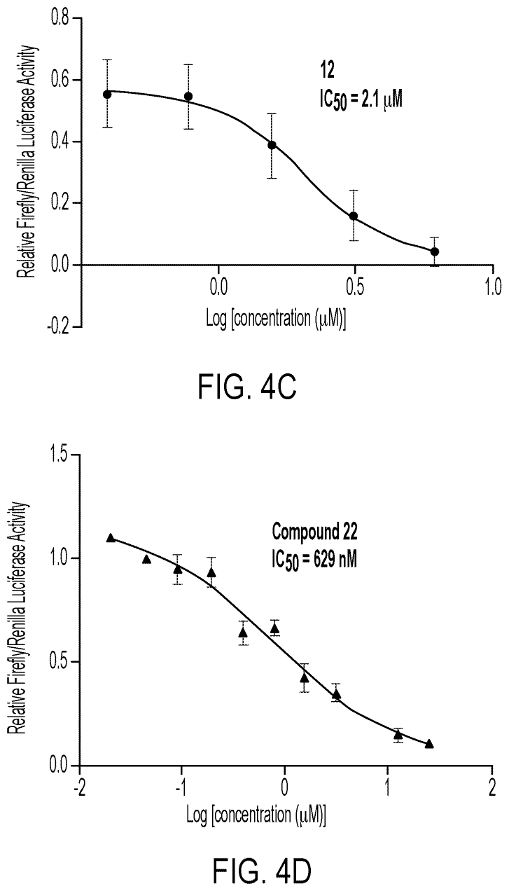

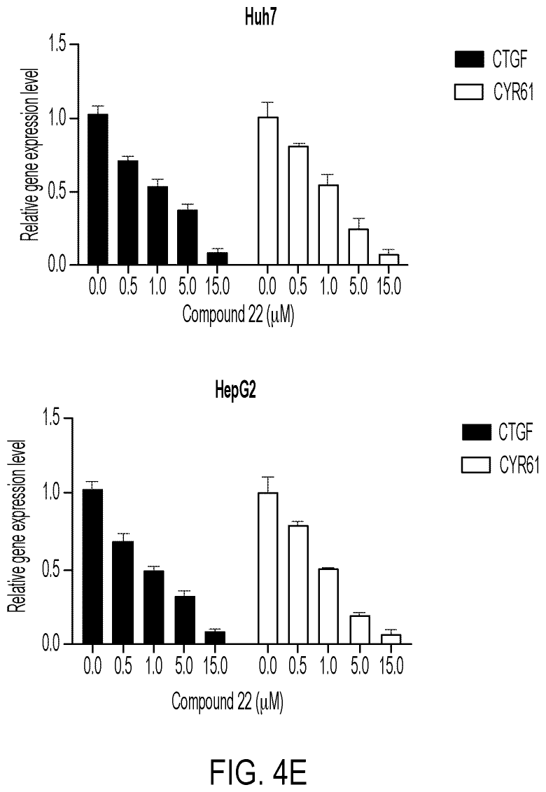

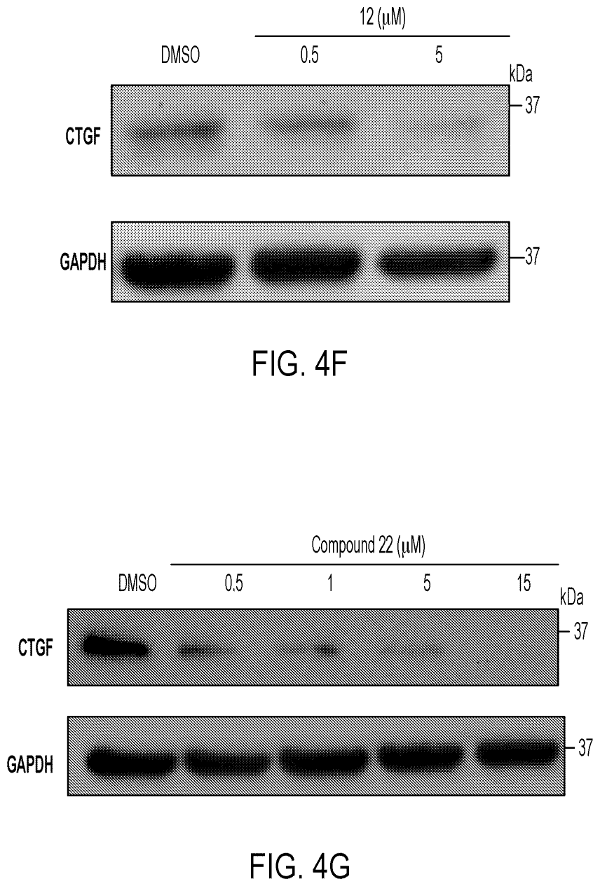

Through the SAR exploration at the R¹, R², R³ parts of the indole core, a class of highly potent inhibitors in a Huh7 cell proliferation assay was identified, as exemplified by compounds 12 and 22. To further investigate the mechanism of the inhibitory effect of compounds in Huh7 cells, TEADs specific luciferase reporter assays were performed with compounds 22 and 12 at several concentrations. As expected based on the results of the cell proliferation assay, the treatment of cells transfected with the 8×GTIIC luciferase reporter with both compounds led to the significant reduction in TEAD reporter activity in a dose dependent manner with IC₅₀ value of 2.1 μM and 629 nM, respectively (FIGS. 4A-4B). TEAD downstream gene expression patterns were evaluated in liver cancer cells Huh7, which has been evidenced with relative high expression of YAP and TEAD2. Consistent with the observed inhibition of TEAD transcriptional activity, the treatment with both compounds suppressed downstream gene CTGF expression and suppression of CTGF protein production in a dose-dependent manner (FIGS. 4F-4G).

### Compound 22 Inhibited Proliferation of Multiple Cancer Cell Lines

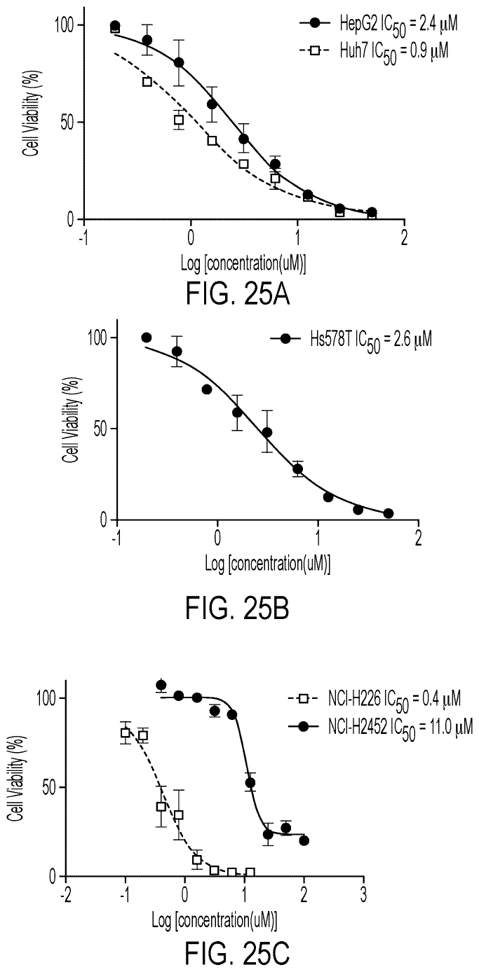

The effect of compound 22 on the best characterized TEAD target genes CTGF and CYR61 were assessed by RT-qPCR. The results showed that the mRNA expression levels for CTGF and CYR61 were significantly inhibited by compound 22 in a dose-dependent manner in the human HCC line Huh7 and the human HB cell line HepG2 respectively (FIG. 4E). In addition, compound 22 demonstrated significant inhibitory effects on the growth of HepG2 cells, with an IC₅₀ value of 2.4 M. This effect was comparatively less pronounced than in the Huh7 cell line, as illustrated in FIG. 25A.

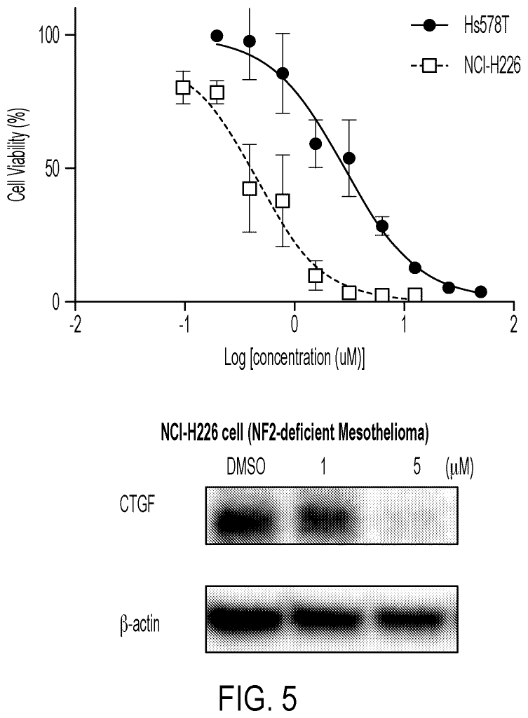

The inhibitory effects of compound 22 were assessed against YAP TEAD related NF2-deficient mesothelioma cell NCI-H226 and triple negative breast cancer cell Hs578T. The growth inhibition ratio was measured using a CCK-8 assay after 3 days of treatments. Results revealed that compound 22 showed antiproliferative activity against both NCI-H226 and Hs578T cancer cell lines with IC₅₀ value of 0.4 μM and 2.6 μM, respectively (FIGS. 5, 25B).

### Covalent Modification of TEAD2-YBD by Compound 22

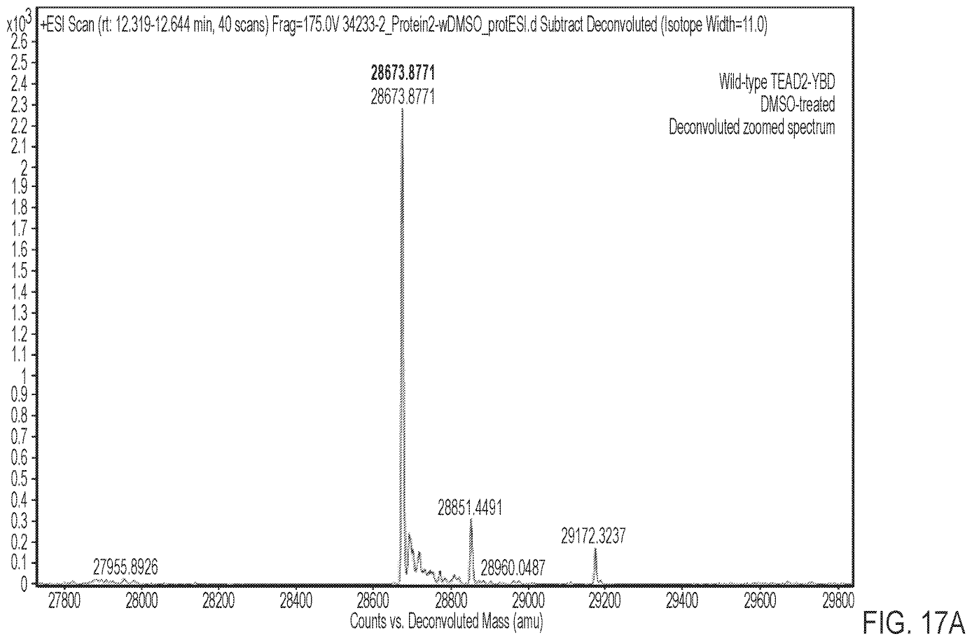

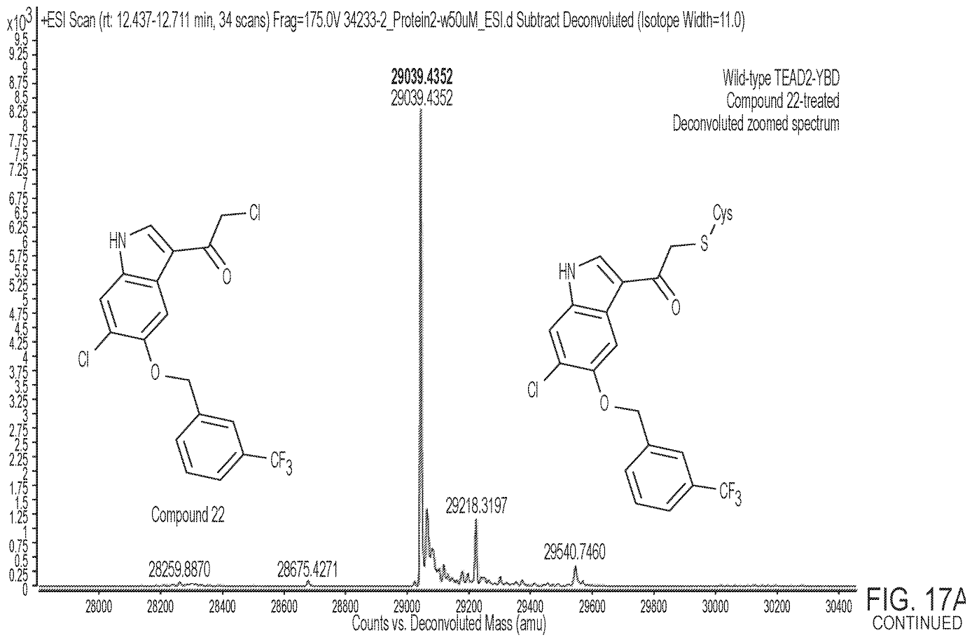

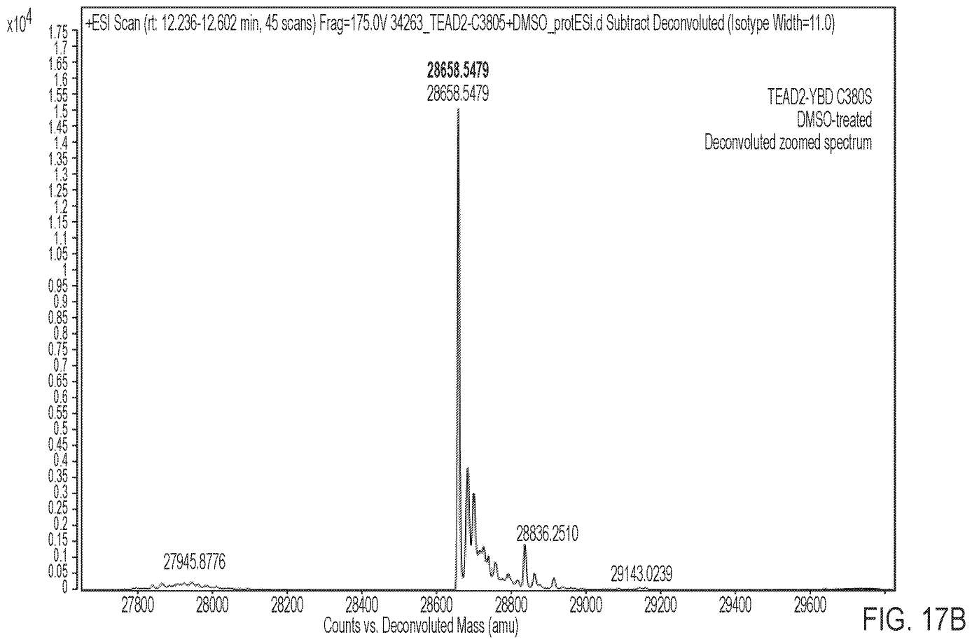

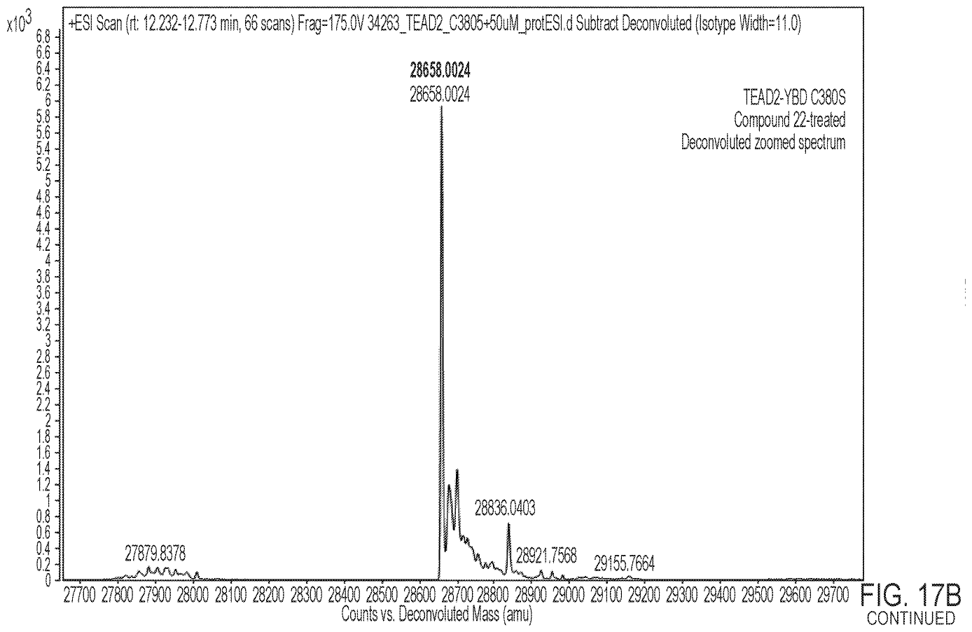

To substantiate the covalent interaction between Compound 22 and TEAD2-YBD, intact mass spectrometry was employed to monitor the protein's mass change upon binding with the compound. Incubation of TEAD2-YBD protein with an excess of compound 22 resulted in an intact mass shift of +365.5581 Da consistent with the formation of a covalent compound 22:TEAD2-YBD complex, attributed to a direct SN2 reaction (FIG. 17A). After 15 hours at room temperature, TEAD2-YBD was completely labeled with a single molecule of compound 22; no evidence of multiple labeling events was detected. Furthermore, the specific interaction with Cys380 was confirmed by the absence of a mass shift when the compound was incubated with the TEAD2-YBD C380S variant for 15 hours, highlighting the targeted modification of TEAD2-YBD by compound 22 at Cys380 (FIG. 17B).

### Compound 22 Disrupts YAP-TEAD Protein-Protein Interaction

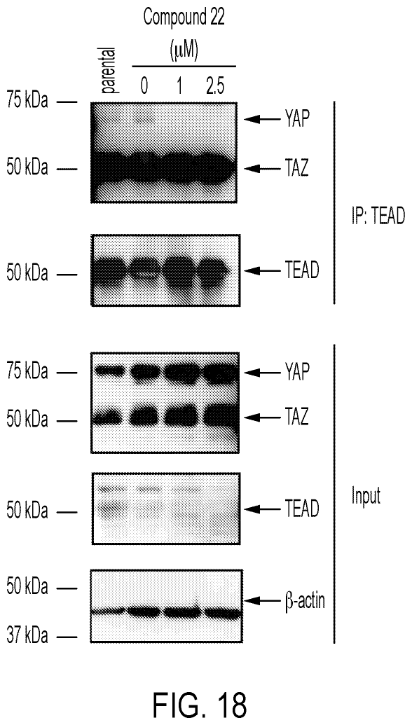

To assess if compound 22 inhibits the interaction between YAP/TAZ and TEAD proteins within the cell, compound 22 was administered to NF2-deficient NCI-H226 cells for 24 hours. Subsequently, the endogenous TEAD protein was isolated through immunoprecipitation using a pan-TEAD antibody and examined the immunocomplexes for the presence of YAP/TAZ proteins using anti-YAP/TAZ antibodies (as depicted in FIG. 18). Treating NCI-H226 cells with 1 μM of compound 22 significantly diminished the co-immunoprecipitation (Co-IP) of YAP, albeit not affecting TAZ, with the pan-TEAD antibody. When the concentration was increased to 2.5 M, the interaction between YAP and TEAD in the NF2-deficient NCI-H226 cells was almost entirely inhibited after a 24-hour treatment period. These results demonstrate that compound 22 effectively and selectively disrupted the binding between YAP and TEAD proteins without impacting the association between TAZ and TEAD.

### Compound 22 Inhibits Huh7 and HepG2 Cell Colony Formation

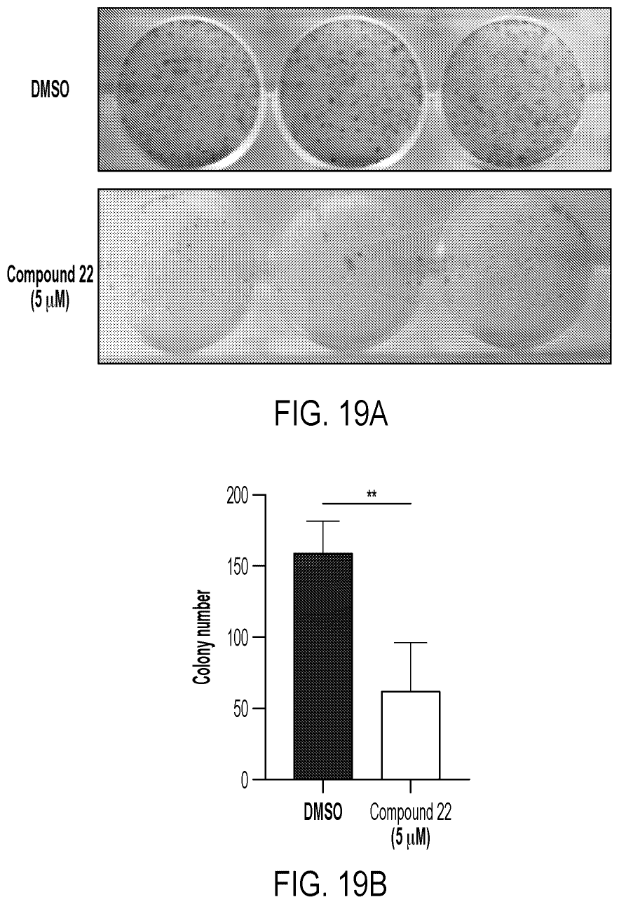

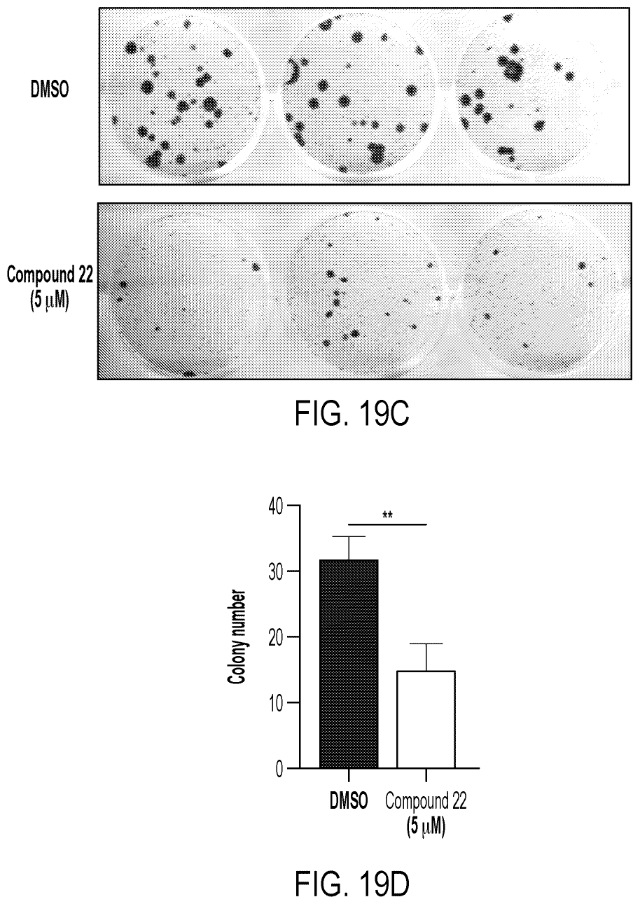

The ability of compound 22 to inhibit the proliferation of liver cancer cell lines Huh7 and HepG2 was further assessed using a clonogenic assay. The clonogenic assay is an in vitro cell survival assay based on the ability of a single cell to grow into a colony.⁷⁷ FIGS. 17A-17B illustrates the ability of compound 22 to inhibit the formation of Huh7 (FIGS. 19A-19B and HepG2 (FIGS. 19C-19D) cell colonies after adding treatment for 48 h, followed by a 10-day incubation with fresh media. Treatment with 5 μM compound 22 led to noteworthy suppression of cell proliferation, with a rate of inhibition amounting to 62.5% for Huh7 cells and 59.5% for HepG2 cells.

### 22 Induces Cell Cycle Arrest in Huh7 and HepG2 Cancer Cells

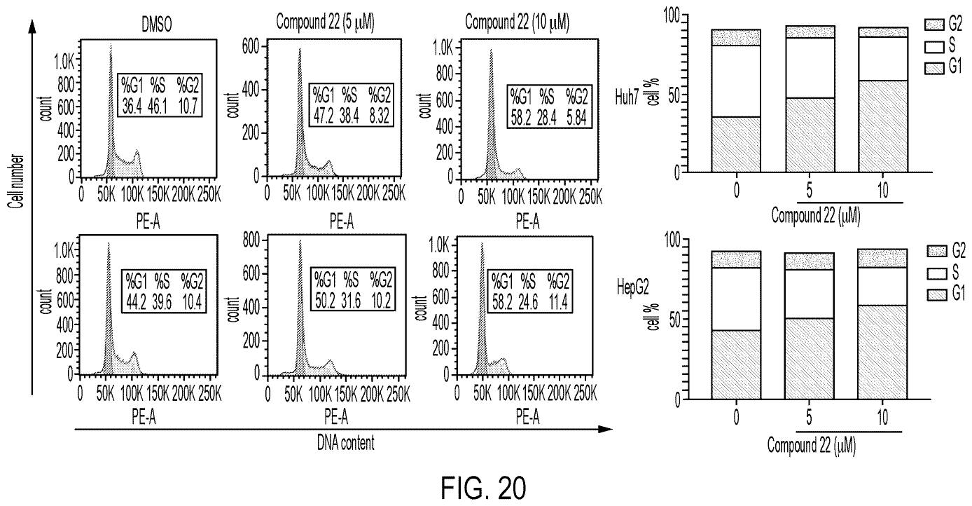

To study the potential mechanistic pathways responsible for cell proliferation inhibition by compound 22, the change of cell cycle in Huh7 and HepG2 liver cancer cell lines was evaluated. DNA-based cell cycle analysis was performed using flow cytometry. The results, as depicted in FIG. 20, indicate that treatment with compound 22 (at doses of 5 and 10 μM for 48 hours) notably increased the proportion of cells in the G1 phase for Huh7 cells, while the proportions of cells in the S/G2 phases experienced a distinct decrease. Specifically, the percentage of cells in the G1 phase after compound 22 treatment was significantly elevated (reaching 58.2%) in comparison to the control group (which had 36.4% in the G1 phase). To explore whether compound 22 also induces G1 phase arrest in HepG2 cells similar to its effects on Huh7 cells, parallel investigations were performed. As illustrated in FIG. 20 compound 22 caused the accumulation of HepG2 cells in the G1 phase at concentrations of 5 and 10 μM. However, the impact of 22 on inducing G1 phase arrest was less pronounced in HepG2 cells compared to Huh7 cells.

### Compound 22 Induces Cell Apoptosis in Huh7 and HepG2 Cancer Cells

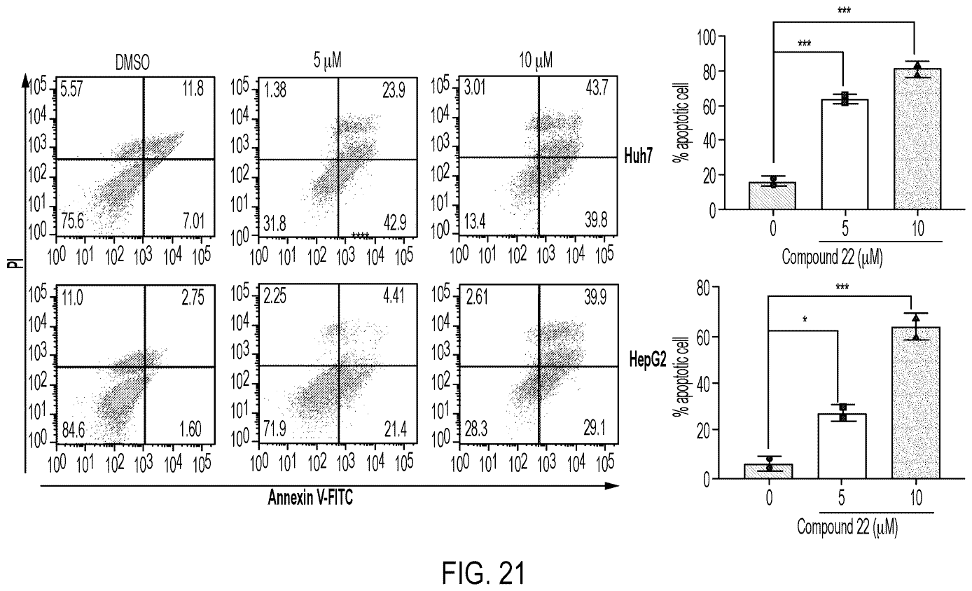

In order to gain deeper insights into the underlying anticancer mechanism of compound 22, experiments were performed to validate whether the induction of G1 phase cells by compound 22 was linked to apoptosis. To achieve this, the cells treated with compound 22 were subjected to PI and FITC-annexin V staining to detect apoptotic cells. As depicted in FIG. 21, cells treated by compound 22 displayed obvious cell apoptosis in Huh7 cells in a dose-dependent manner. The proportions of total apoptosis including the early stage apoptosis and the later stage of apoptosis were 66.8%, and 83.5%, respectively, at concentrations of 5 and 10 μM. This trend was similarly observed in the case of HepG2 cells. The maximum apoptotic rate (early+late apoptotic) at the concentration of 10 μM was achieved at 69.0%. The impact of compound 22 on inducing cell apoptosis was less pronounced in HepG2 cells compared to Huh7 cells, aligning with the outcomes of the cell cycle analysis, providing further coherence to the observed effects of compound 22 on these cell lines.

In vivo Antitumor Evaluation of Compound 22 in the Huh7 Mouse Xenograft Model Shows Compound 22 Represses Tumor Growth

Based on the favorable in vitro potency and low toxicity, the in vivo antitumor efficacy of compound 22 was further evaluated in the NSG mouse (NOD scid gamma mouse) xenograft model from human Huh7 cells. Sorafenib, the first-line treatment for primary hepatocellular carcinoma (HCC) patients, served as the control. Compound 22 was tested as a monotherapy and in combination with Sorafenib.

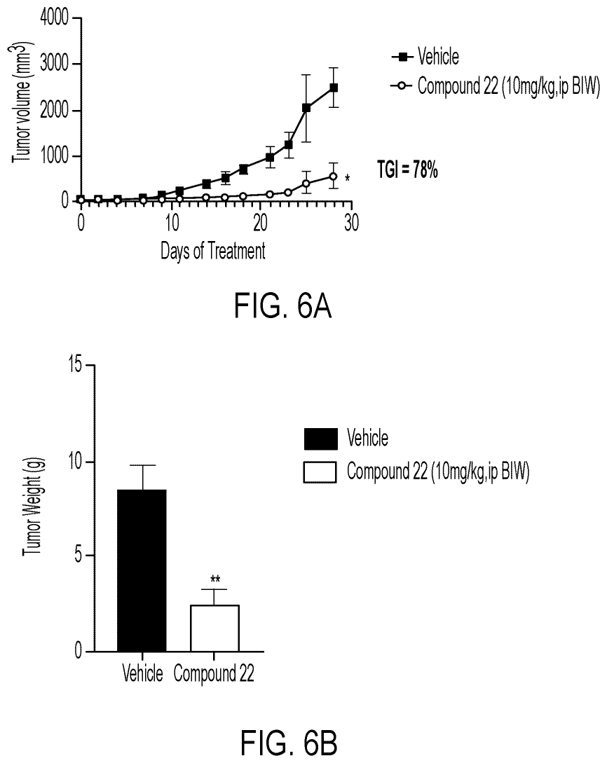

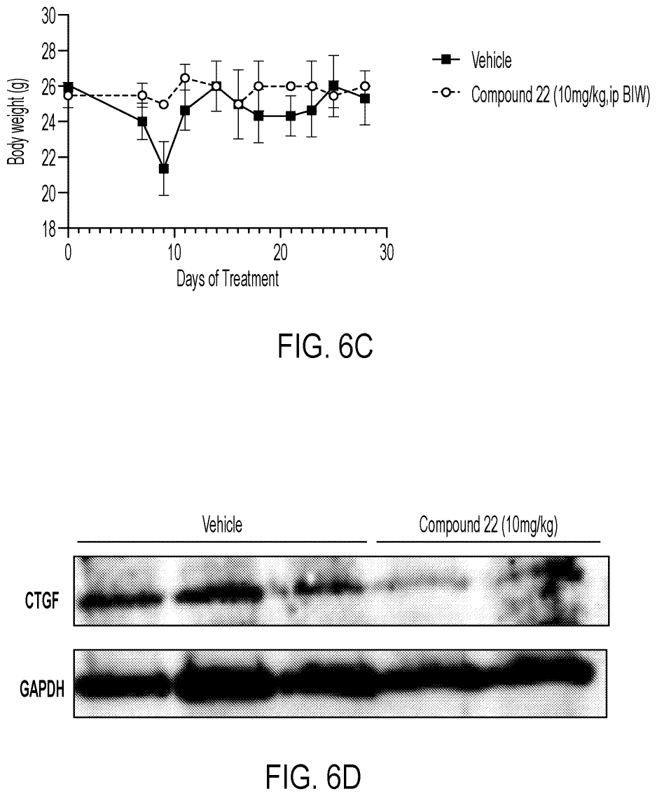

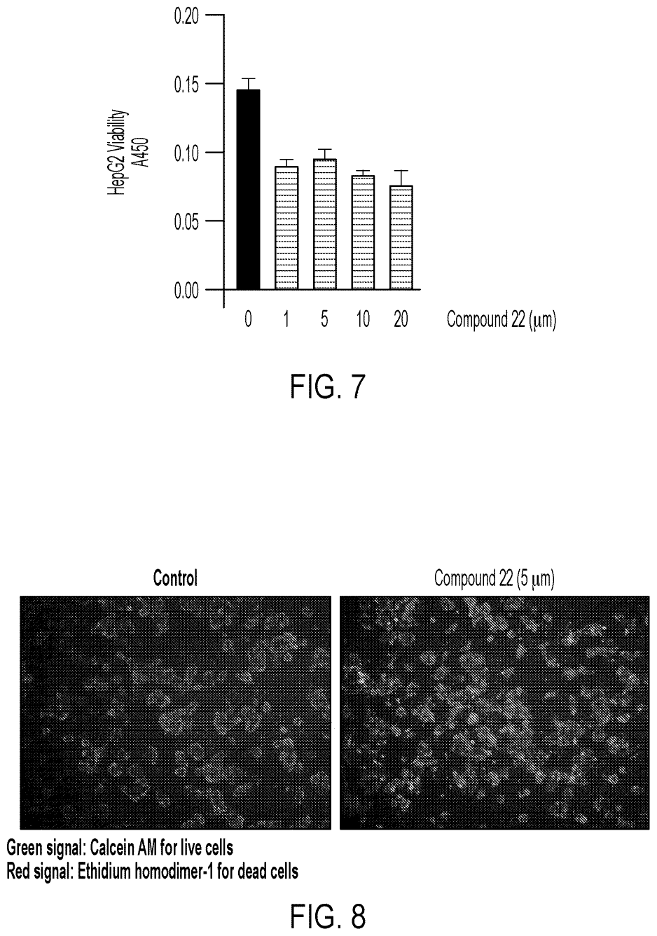

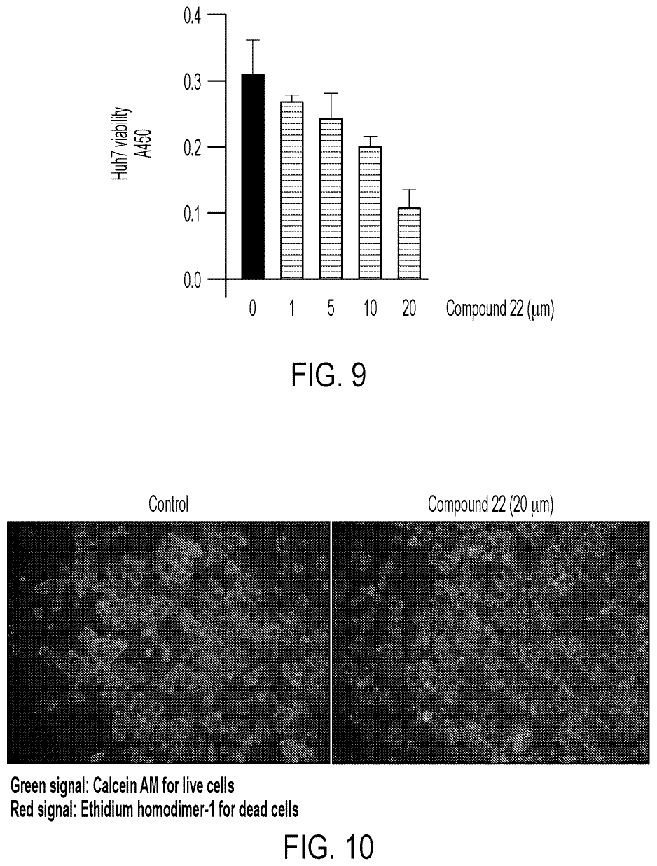

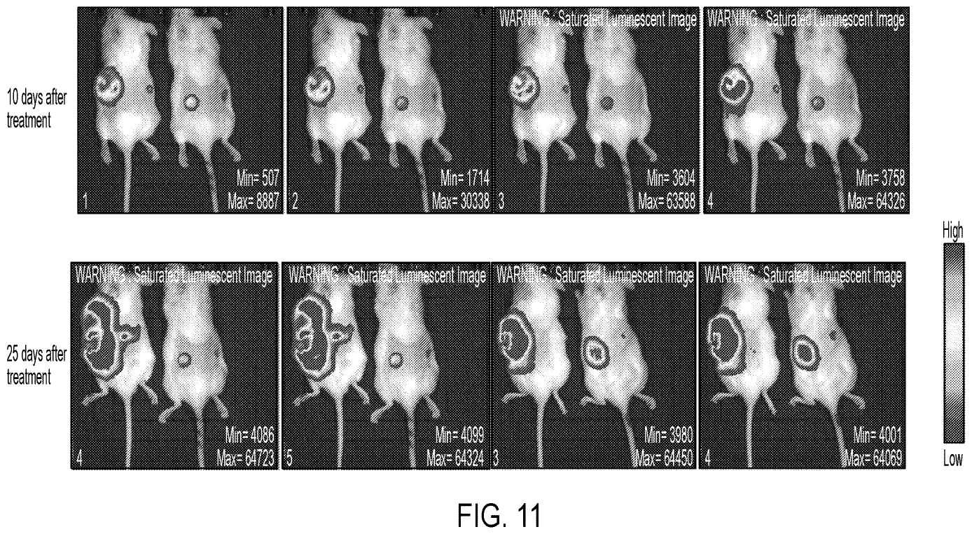

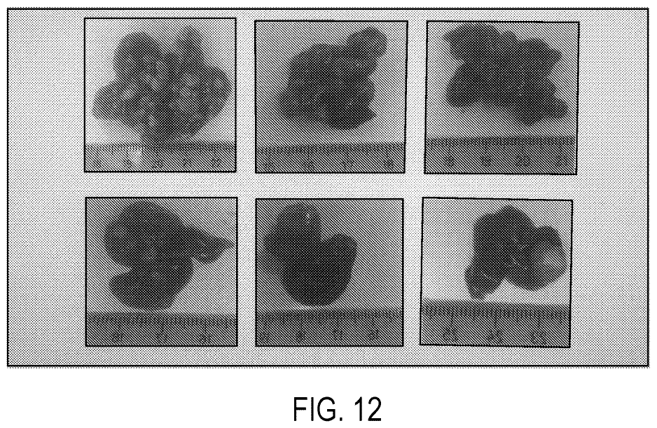

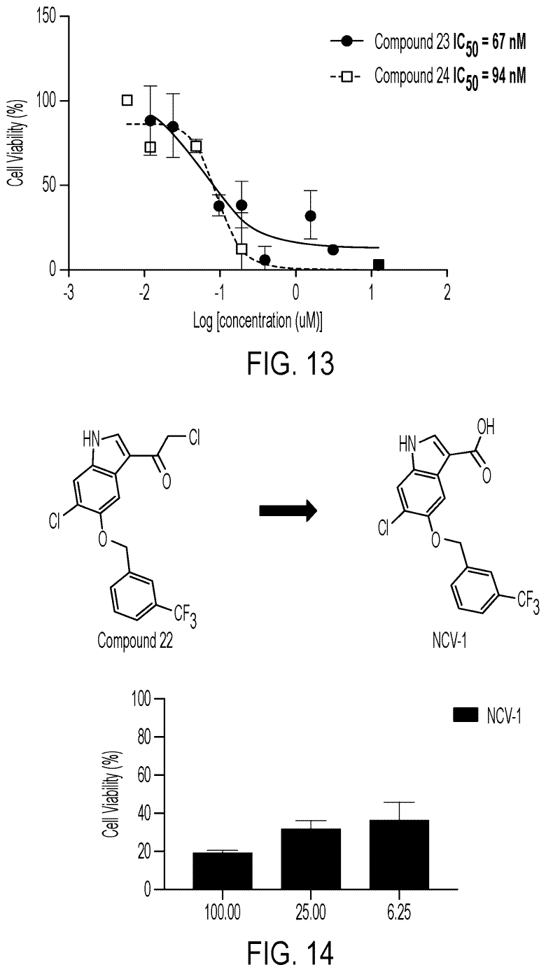

The animals were administered 10 mg/kg of compound 22 twice a week for four weeks via intraperitoneal (IP) injection. Compound compound 22 showed potent antitumor efficacy with a tumor growth inhibition (TGI) rate of 78% (FIGS. 6A-6B).

The mice were administered 50 mg/kg of compound 22 every three day for 24 days via intraperitoneal (IP) injection. Sorafenib was administered orally at a dose of 100 mg/kg following the same schedule.

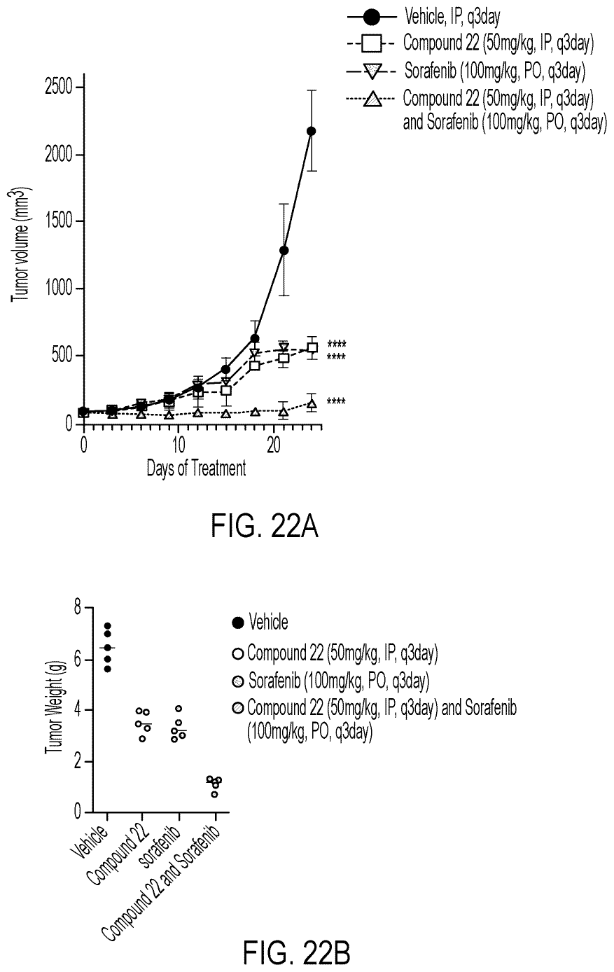

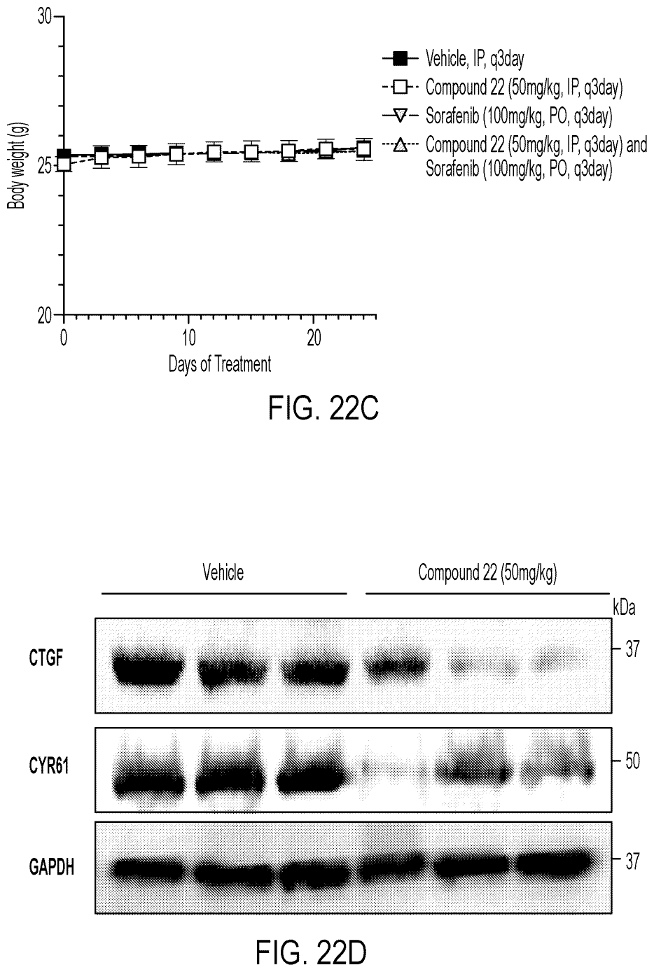

As demonstrated in FIGS. 22A-22B, compound 22 showed potent antitumor efficacy with a tumor growth inhibition (TGI) rate of 75%, similar to that of Sorafenib, which had a TGI of 76%. Importantly, the combination therapy of compound 22 and Sorafenib resulted in a more substantial tumor growth inhibition, with a TGI rate of 93%. No significant change of the body weight was observed (FIG. 6C), indicating that there was no apparent toxicity upon the treatment of compound 22 in the xenograft model.

To elucidate the mechanism of compound 22-mediated antitumor activity in vivo, the expression level of CTGF and CYR61 in the tumor tissue were examined by western blotting. A remarkable decrease of CTGF and CYR61 was observed in the compound 22-treated groups than that in the vehicle groups (FIG. 6D). Without wishing to be bound by any particular theory, these results revealed that compound 22 may suppress Huh7 xenograft tumor growth in vivo by inhibiting the TEAD transcription.

### In Vivo Antitumor Evaluation of Compound 22 in Mouse Models

Hepatoblastoma (HB) is the most common type of childhood liver cancer and usually affects children younger than 3 years of age. This type of primary liver neoplasm often contains activation of both Wnt/β-catenin and YAP signaling in about 80% of the HB cases. The transcriptional coactivator YAP has been shown to cooperate with β-catenin to promote HB. Co-expression of constitutively active S127A-YAP1 and ΔN90 deletion-mutant β-catenin (YAP1-ΔN90-β-catenin) via Sleeping Beauty-mediated somatic integration causes HB in mouse livers (PMID:30794807).⁷⁸

Using this model, the inhibitory effect of YAP inhibitor compound 22 on HB development was examined. In brief, following HDTV injection, the co-expression of constitutively active S127A-YAP1 and ΔN90 deletion-mutant β-catenin (ΔN90-β-catenin) via Sleeping Beauty-mediated somatic integration led to HB development in mouse livers. Plasmids carrying T3EF1α-YAPS127A (20 μg), pCMV-sleeping beauty transposase (SB, 0.8 μg), and pT3-EF1α-ΔN90-β-catenin (20 μg) were extracted using the Endotoxin-Free Maxiprep kit (Qiagen), diluted in 2 ml 0.9% sodium chloride solution, and injected via hydrodynamic tail vein injection (HDTV) into C57BL6 mice (6 week-old, body weight of 20 g). compound 22 or mock control (twice a week for three months, IP injection) were applied two weeks post the HDTV injection until two days before sacrifice. Low dose group received 50 mg/g body weight and high dose group received 125 mg/g body weight.

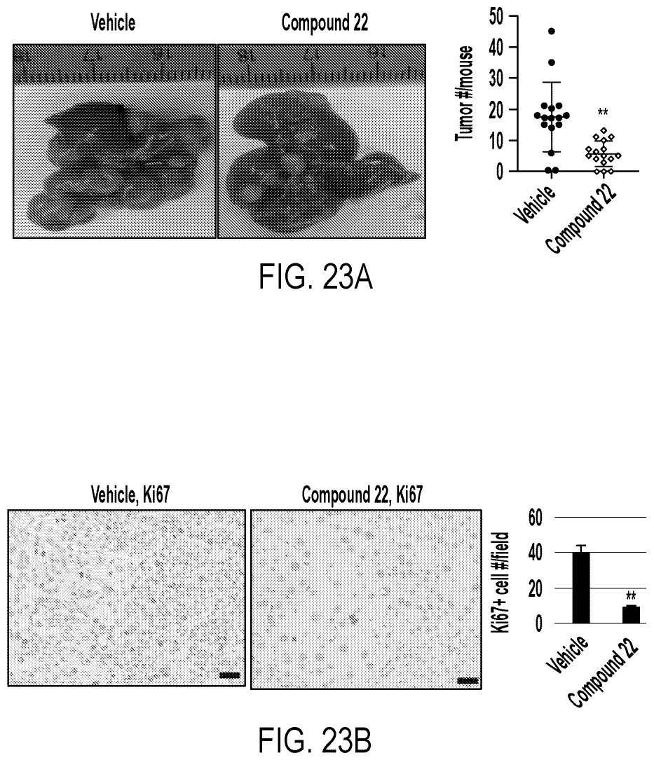

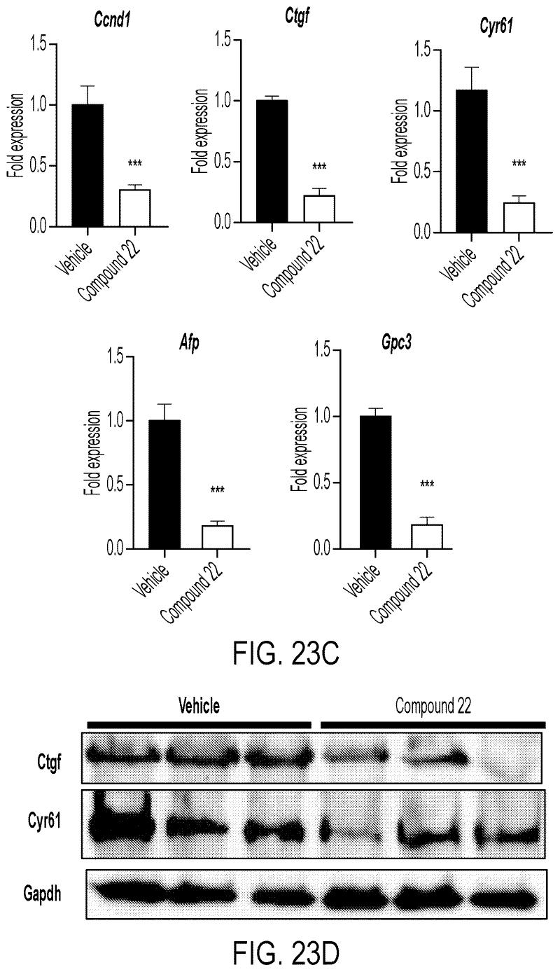

There were about 18 HB tumors per mouse on the average at three months post the co-expression of S127A-YAP1 and ΔN90-β-catenin (FIG. 23A). However, upon administering compound 22 (25 mg/kg body weight, twice a week for 9 weeks), there was a notable reduction in both the occurrence and size of HB tumors. IHC for the cell proliferation marker Cyclin D1 also showed decreased number of proliferating cells in presence of compound 22 treatment (FIG. 23B). Real time qRT-PCR analyses indicated lower levels of Ccnd1, CTGF, Cyr61, Afp, and Gpc3 mRNAs (FIG. 23C). Western blotting confirmed decreased CTGF and Cyr61 in the compound 22-treated livers (FIG. 23D). Taken together, these data indicated that compound 22 inhibited HB tumor growth.

### CONCLUSIONS

Based on a combination of structure-based design following a virtual screen and biological evaluation, C-3 was identified as a moderate TEAD transcriptional inhibitor. Further rational chemical optimization led to the identification of compound 22 as a potent covalent TEAD inhibitor bearing a previously unexplored chemotype with the Huh7 cell proliferation IC₅₀ value of 0.9 M. Also, compound 22 inhibited TEADs transcription activity leading to downregulation of gene expression downstream of YAP signaling. Compound 22 demonstrated the ability to dose-dependently hinder colony formation, provoke apoptosis, and induce cell cycle arrest in both Huh7 and HepG2 cells. Compound 22 (TGI=78%) inhibited tumor growth significantly in the Huh7 mouse xenograft model, which was accompanied by CTGF and CYR61 downregulation in the tumor tissue.

Compound 22 also displayed potent inhibition of hepatoblastoma development in mice induced by hydrodynamic injection of activated forms of oncogenic YAP and β-catenin proteins. All the above results manifest that compounds provided herein, including compound 22, are strong TEAD inhibitors with activity against hepatocellular carcinoma and HB.

### Experimental Section

### Chemistry.

Compounds of any of the formulae herein can be synthesized using reagents and chemistry methodology known in the art, including in references described herein and in experimental details herein. The preparation of compounds 1-3 is illustrated in Scheme 1. The benzyl ether compound 1a underwent vicarious nucleophilic substitution in the presence of potassium tert-butoxide to give compound 3a. Treatment with palladium on carbon under a hydrogen atmosphere reduced the nitro functionality, cyclized the indole heterocyclic ring and removed the O-benzyl protection, to obtain compound 4a. The free hydroxyl was subjected to an S_{N}2 reaction with different benzyl bromides to give the corresponding ether compounds, which subsequently treated with phosphoryl chloride and 2-chloro-N,N-dimethylacetamide to produce the final products.

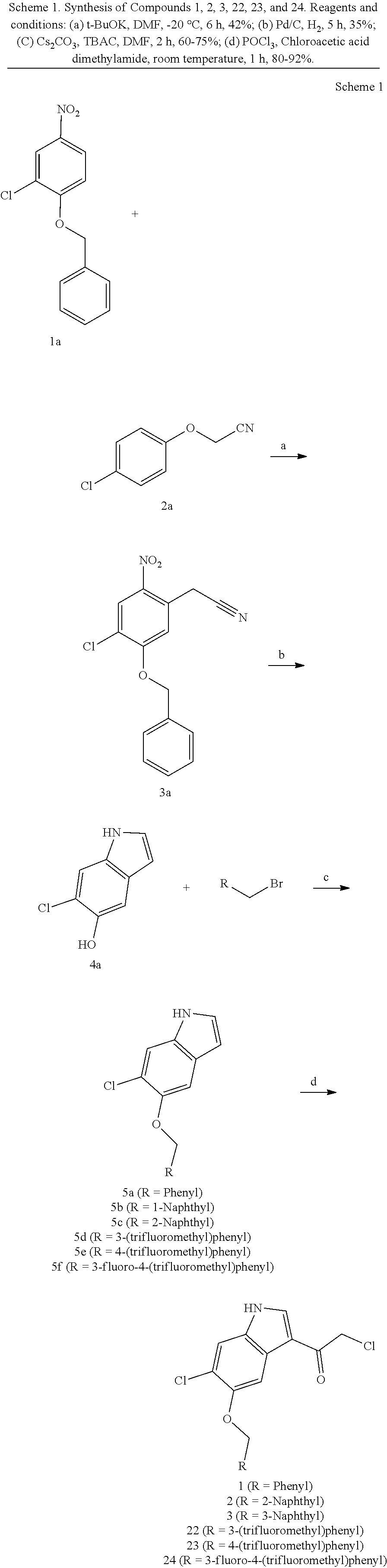

Synthetic routes for preparing compounds 5, 4 and 6 are outlined in Scheme 2. 4-Nitro-2-methoxyphenol (6a) was protected as the benzyl ether and converted to the compound 7a which underwent vicarious nucleophilic substitution in the presence of potassium tert-butoxide to give compound 8a. Treatment with palladium on carbon under a hydrogen atmosphere reduced the nitro functionality, cyclized the indole heterocyclic ring and removed the O-benzyl protection, to obtain compound 9a. The hydroxyl was subjected to S_{N}2 reaction with different benzylic bromides to give corresponding ether compounds 10a, 10b and 10c, and subsequently treated with treated with phosphoryl chloride and 2-chloro-N,N-dimethylacetamide to produce the final products.

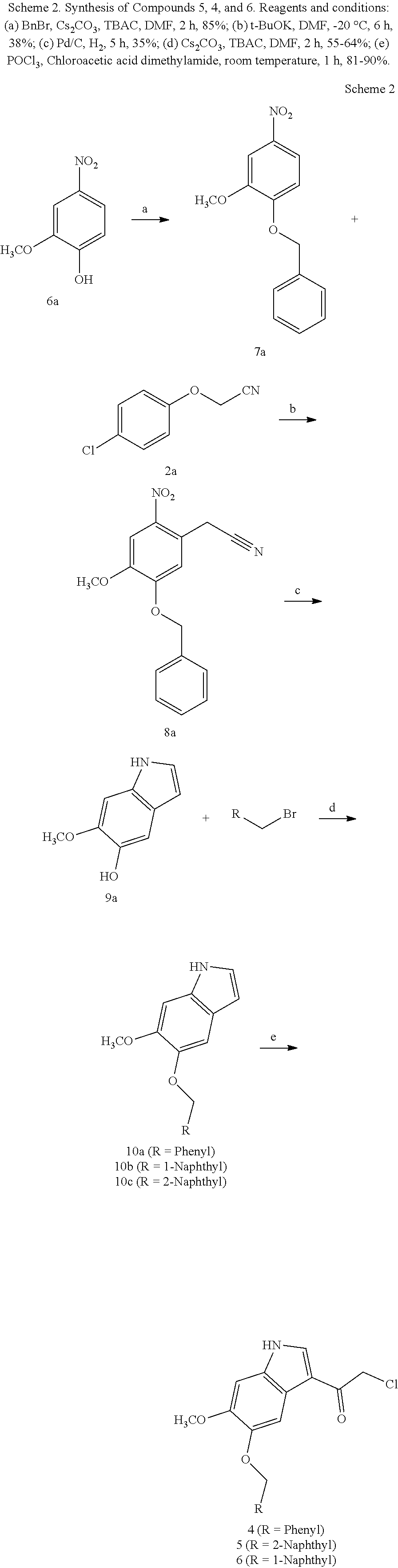

The preparation of compound 8 without substitution at indole 6 position is shown in Scheme 3. The commercially available compound 11a was reacted with phosphoryl chloride and 2-chloro-N,N-dimethylacetamide to provide the final product 8 in high yield.

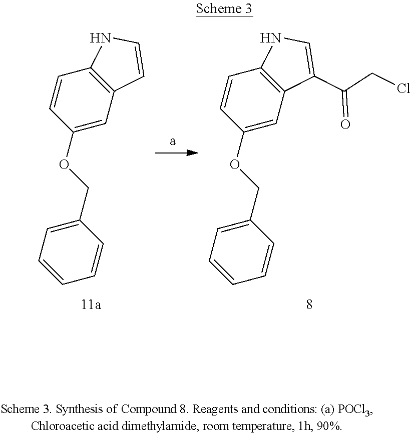 [SMILES: c1ccc(COc2ccc3[nH]ccc3c2)cc1 ; O=C(CCl)c1c[nH]c2ccc(OCc3ccccc3)cc12]

The preparation of compound 7 is illustrated in Scheme 4. The commercially available compound 12a was deprotected via palladium-catalyzed hydrogenolysis to give the corresponding phenol 13a. The hydroxyl was subjected to S_{N}2 reaction with 2-(bromomethyl)naphthalene to give corresponding ether compound 14a, and subsequently treated with treated with phosphoryl chloride and 2-chloro-N,N-dimethylacetamide to produce the final product 7.

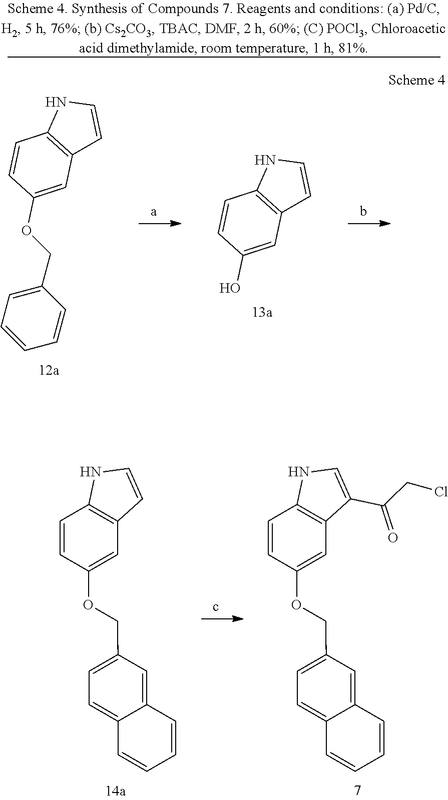 [SMILES: c1ccc(COc2ccc3[nH]ccc3c2)cc1 ; Oc1ccc2[nH]ccc2c1 ; c1ccc2cc(COc3ccc4[nH]ccc4c3)ccc2c1 ; O=C(CCl)c1c[nH]c2ccc(OCc3ccc4ccccc4c3)cc12]

The preparation of compounds 9-11 is shown in Scheme 5. From compounds 5a, 5b and 5c, Vilsmeier Haack reaction added an aldehyde moiety at the indole 3 position to give 15a, 15b, and 15c. Then condensation with hydroxylamine hydrochloride furnished the corresponding oximes, which were subjected to zinc reduction to offer amines 17a, 17b and 17c. Then coupling with acryloyl chloride in the presence of triethylamine provided the final compounds 9, 10, and 11.

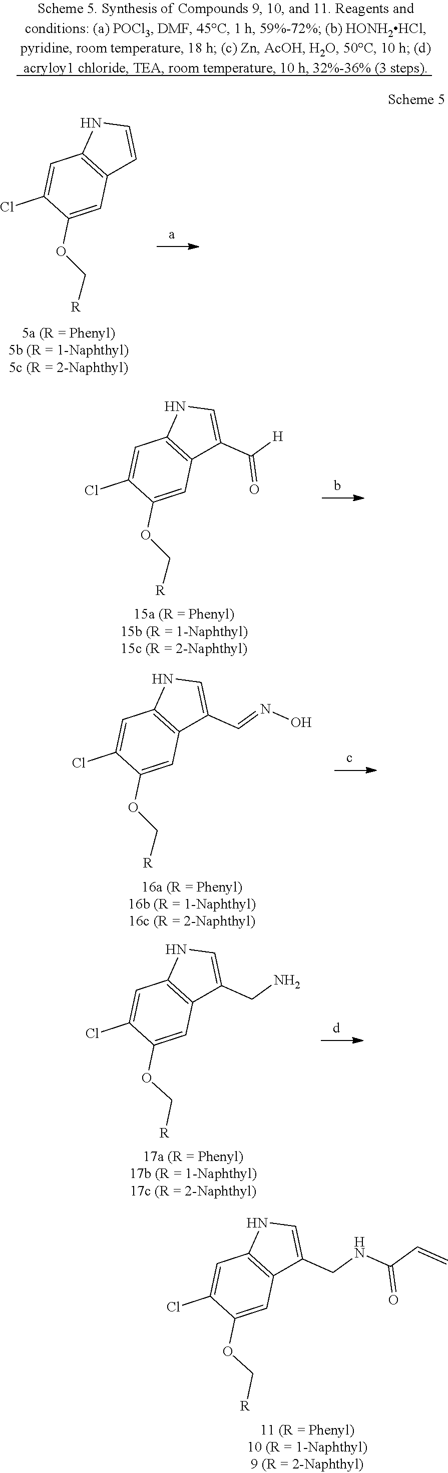

The preparation of compounds 12, 13, 14 and 15 is shown in Scheme 6. From benzyl ether compound 1a, six steps afforded the corresponding amines 17a, 17b, 17c and 17d. Then coupling with 2-chloroacetyl chloride furnished the final products 12, 13, 14 and 15.

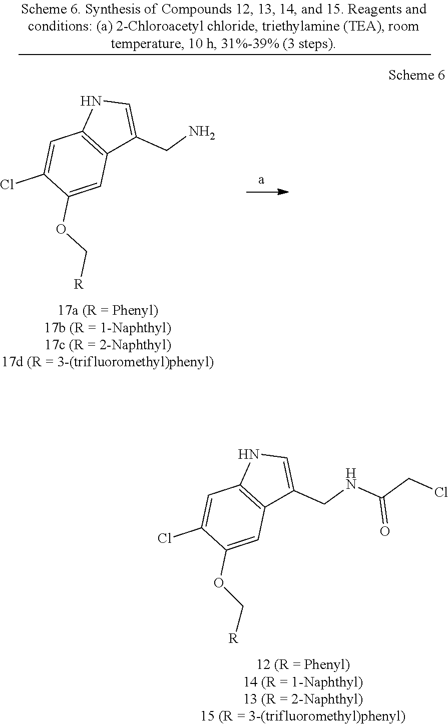

The preparation of compounds 16-20 and 21 is shown in Scheme 7. From benzyl ether compound 1 four steps offered the corresponding aldehydes 15a-15f. Then nucleophilic addition with 2-cyanoacetamide provided the final products 16-21.

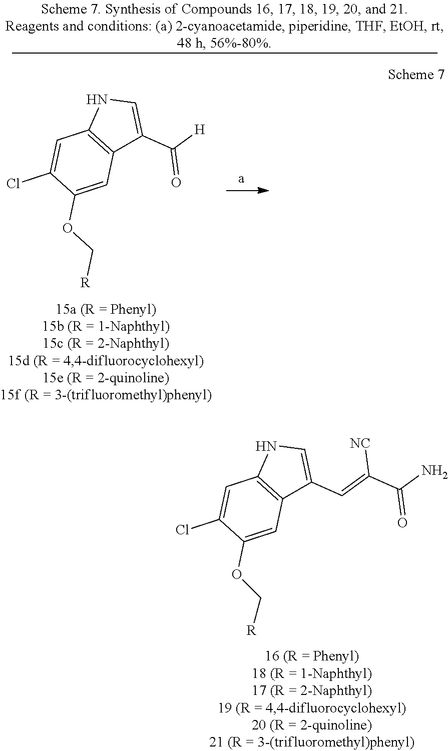

Synthetic route of compound 22 is described in Scheme 1. From benzyl ether compound 1a two steps provided the phenol compound 4a, which was then subjected to S_{N}2 reaction with bromide to get compound 5d. Then 5d was reacted with phosphoryl chloride and 2-chloro-N,N-dimethylacetamide to get the attachment of the chloroacetone.

25 was prepared as outlined in Scheme 8. From benzyl ether compound 1, six steps afforded the corresponding amines 17a. Then coupling with carboxylic acid 18a using HATU/DIPEA provided the final product in good yield.

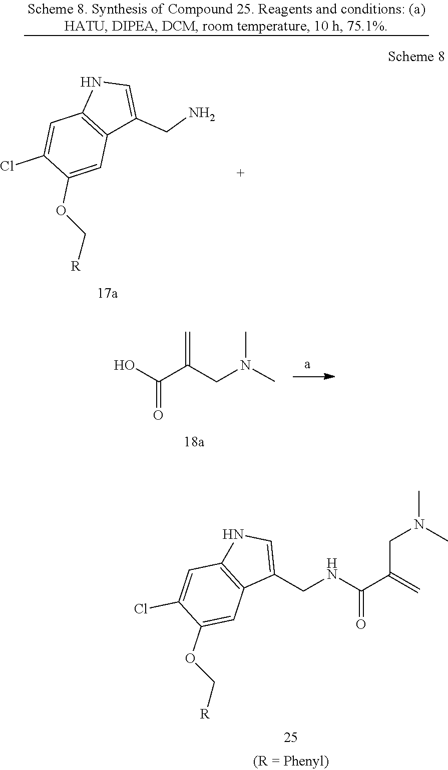

### Chemical Reagents and General Method.

All commercially available starting materials and solvents were reagent grade and used without further purification. Purification by silica gel column chromatography was carried out using a Biotage Isolera One purification systems with prepacked cartridges.

Analytical thin layer chromatography (TLC) was performed on silica gel plates and visualized under ultraviolet light (254 nm) for monitoring reactions. ¹H and ¹³C NMR spectra were recorded on a Bruker Avance NEO-600 spectrometer or Bruker Avance III HD 600 (¹H NMR: 600 MHz for proton (¹H NMR); and ¹³C NMR: 151 MHz) using DMSO-d₆, CD₃OD, or CDCl₃ as solvents. Chemical shifts (6 values) are expressed in ppm using tetramethyl silane as an internal standard (TMS ¹H NMR: 0.00 ppm, ¹³C NMR: 0.00 ppm), and the coupling constants (J values) are indicated in hertz (Hz). Low-resolution mass spectra for certain known compounds were obtained from the University of Florida Department of Medicinal Chemistry 3200 QTrap LC/MS/MS spectrometer via direct injection, and high-resolution mass spectra were obtained from the Mass Spectrometry Facility within the University of Florida Department of Medicinal Chemistry Thermo Scientific™ Orbitrap Focus mass spectrometer with Dionex™ Ultimate™ 3000 UHPLC system. The purity of all target compounds was determined to be greater than 95%, ascertained using high-performance liquid chromatography (HPLC). This analysis was conducted on an Agilent 1100 Series HPLC instrument equipped with an Acclaim 120 C18 column (2.1 mm×100 mm, 5 m). For the HPLC, H2O containing 0.1% formic acid (FA) was used as solvent A, and MeOH was used as solvent B. The flow rate was set at 0.3 mL/min. Detection was carried out at a UV wavelength of 254 nm. The gradient elution program proceeded as follows: from 20% to 100% B over 7 minutes, held at 100% B for 8 minutes, from 100% B to 20% B in 0.1 minute, and then held at 10% B for 3.9 minutes.

### 2-(5-(benzyloxy)-4-chloro-2-nitrophenyl)acetonitrile (3)

A solution of 1-(benzyloxy)-2-chloro-4-nitrobenzene 1a (500 mg, 1.90 mmol) and (4-chlorophenoxy)-acetonitrile 2a (349.6 mg, 2.09 mmol, 1.1 equiv) in anhydrous DMF (4 mL) was added dropwise to a stirred solution of potassium tert-butoxide (468.13 mg, 4.17 mmol, 2.2 equiv) in anhydrous DMF (6 mL) under nitrogen at −10 ° C The resultant deep purple solution was stirred at −10 ° C under nitrogen for 5 h, at which time an aqueous solution of 5% HCl (15 mL) was added dropwise and then extracted with ethyl acetate (30 mL×3). The organic layers were combined, washed with brine, and dried over anhydrous Na₂SO₄. After filtration, the solvent was removed under vacuum and the residue was purified by silica gel column chromatography (EtOAc:hexane=1:4) to afford compound 3a (241.7 mg, 42.1%) as a white solid. ¹H NMR (600 MHz, Chloroform-d) δ 8.34 (s, 1H), 7.51-7.46 (m, 2H), 7.46-7.41 (m, 2H), 7.41-7.35 (m, 1H), 7.28 (s, 1H), 5.33 (s, 2H), 4.24 (s, 2H). ¹³C NMR (151 MHz, Chloroform-d): δ 158.6, 139.9, 134.4, 129.0, 128.8, 128.4, 127.4, 126.8, 123.8, 116.2, 114.2, 71.7, 23.3. ESI-MS (m/z) cal. for C₁₅H₁₂ClN₂O₃ μM+H]+: 303.05; found 303.09.

### 6-chloro-1H-indol-5-ol (4)

A mixture of 3a (200 mg, 0.66 mmol) and Pd/C (10 mg, 10 wt. % Pd) in ethanol (5 mL) in a reaction vial was equipped with a H₂ balloon and stirred at 25 ° C for 5 h. Upon completion of the reaction, the mixture was filtered, and the EtOH was removed under vacuum. The residue was further purified by silica gel column chromatography (EtOAc:hexane=1:2) to obtain compound 4a (38.8 mg, 35.1%) as a white solid. ¹H NMR (600 MHz, Chloroform-d) δ 8.01 (s, 1H), 7.38-7.36 (m, 1H), 7.24 (s, 1H), 7.20-7.17 (m, 1H), 6.44 (m, 1H), 5.25 (s, 1H). ¹³C NMR (151 MHz, Chloroform-d) δ 145.2, 130.7, 127.9, 125.8, 116.2, 110.8, 105.7, 102.4. ESI-MS (m/z) cal. for C₈H₅ClNO [M−H]+: 166.01; found 166.06.

### 1H-indol-5-ol

A mixture of 12a (200 mg, 0.90 mmol) and Pd/C (10 mg, 10 wt. % Pd) in Ethanol (5 mL) was stirred in a reaction vial equipped with a H₂ balloon at 25 ° C for 2-5 h. The reaction progress was monitored by thin layer chromatography (TLC). The mixture was filtered upon completion. The EtOH was removed under vacuum. The residue was further purified by silica gel column chromatography (EtOAc:hexane=1:2 or 1:5) to obtain compound 13a (38.8 mg, 35.1%) as a white solid or (91.2 mg, 76.1%) as a grey solid. ¹H NMR (600 MHz, Chloroform-d) δ 8.01 (s, 1H), 7.38-7.36 (m, 1H), 7.24 (s, 1H), 7.20-7.17 (m, 1H), 6.44 (m, 1H), 5.25 (s, 1H). ¹³C NMR (600 MHz, Chloroform-d) δ 145.19, 130.74, 127.92, 125.84, 116.15, 110.78, 105.72, 102.35. ¹H NMR (600 MHz, Methanol-d4): δ 7.19 (d, J=8.6 Hz, 1H), 7.14 (d, J=3.1 Hz, 1H), 6.93 (d, J=2.1 Hz, 1H), 6.66 (dd, J=8.6, 2.3 Hz, 1H), 6.27 (dd, J=3.0, 0.7 Hz, 1H). ¹³C NMR (151 MHz, Methanol-d4): δ 151.3, 132.6, 130.1, 126.2, 112.4, 112.2, 105.2, 101.6. ESI-MS (m/z) cal. for C₈H₆NO [M−H]+: 132.04; found 132.01.

### 5-(benzyloxy)-6-chloro-1H-indole (5a)

To a solution of compound 4a (200 mg, 1.19 mmol) in N,N-dimethylformamide (6 ml) was added benzyl bromide (184.3 μL, 1.55 mmol), cesium carbonate (777.66 mg, 2.38 mmol) and tetrabutylammonium chloride (1.33 g, 4.76 mmol), The resultant reaction mixture was stirred at 25 ° C The reaction was monitored by TLC. On completion of the reaction, the reaction mixture was quenched with water and extracted with ethyl acetate (3×30 mL). The combined organic layers were washed with brine, dried over anhydrous Na₂SO₄ and concentrated under reduced pressure. The resultant residue was used for the next step without purification as a mixture. Alternatively, the residue was purified by silica gel column chromatography (EtOAc:hexane=1:5) to obtain compound 5 (200.6 mg, 65.4%) as a white solid. ¹H NMR (600 MHz, Chloroform-d) δ 8.04 (s, 1H), 7.52 (d, J=7.6 Hz, 2H), 7.45-7.43 (m, 1H), 7.39 (t, J=7.6 Hz, 2H), 7.32 (t, J=7.4 Hz, 1H), 7.21 (s, 1H), 7.19-7.15 (m, 1H), 6.45 (ddd, J=3.0, 2.0, 0.9 Hz, 1H), 5.17 (s, 2H). ¹³C NMR (151 MHz, Chloroform-d): δ 148.7, 137.2, 130.8, 128.5, 127.8, 127.2, 126.9, 125.2, 119.5, 112.4, 105.6, 102.6, 71.9. ESI-MS (m/z) cal. for C₁₅H₁₁ClNO [M−H]+: 256.05; found 256.12.

### 6-chloro-5-(naphthalen-1-ylmethoxy)-1H-indole (5b)

6-chloro-5-(naphthalen-1-ylmethoxy)-1H-indole was synthesized in a similar manner as described for compound 5a. White solid, 68.4% yield. ¹H NMR (600 MHz, Chloroform-d): δ 8.16 (d, J=8.4 Hz, 1H), 8.03 (s, 1H), 7.90 (d, J=7.6 Hz, 1H), 7.84 (d, J=8.2 Hz, 1H), 7.71 (d, J=6.4 Hz, 1H), 7.56 (ddd, J=8.4, 6.8, 1.4 Hz, 1H), 7.52 (ddd, J=8.0, 6.9, 1.2 Hz, 1H), 7.47 (dd, J=8.1, 7.1 Hz, 1H), 7.45-7.43 (m, 1H), 7.30 (s, 1H), 7.17-7.14 (m, 1H), 6.45 (ddd, J=3.0, 2.0, 0.8 Hz, 1H), 5.60 (s, 2H). ¹³C NMR (151 MHz, Chloroform-d): δ 148.8, 133.7, 132.5, 131.3, 130.9, 128.7, 128.6, 126.9, 126.3, 126.1, 125.8, 125.4, 125.3, 123.7, 119.7, 112.4, 105.8, 102.6, 70.5. ESI-MS (m/z) cal. for C₁₉H₁₃ClNO [M−H]+: 306.07; found 306.01.

### 6-chloro-5-(naphthalen-2-ylmethoxy)-1H-indole (5c)

6-chloro-5-(naphthalen-2-ylmethoxy)-1H-indole was synthesized in a similar manner as described for compound 5a. White solid, 64.2% yield. 1H NMR (600 MHz, Chloroform-d): δ 8.02 (s, 1H), 7.95 (s, 1H), 7.89-7.81 (m, 3H), 7.63 (dd, J=8.4, 1.6 Hz, 1H), 7.50-7.45 (m, 2H), 7.44-7.41 (m, 1H), 7.24 (s, 1H), 7.14 (dd, J=3.2, 2.6 Hz, 1H), 6.42 (ddd, J=3.0, 2.0, 0.8 Hz, 1H), 5.32 (s, 2H). ¹³C NMR (151 MHz, Chloroform-d): δ 148.7, 134.8, 133.3, 133.1, 130.9, 128.3, 128.0, 127.7, 126.9, 126.1, 126.1, 125.9, 125.3, 125.2, 119.6, 112.4, 105.8, 102.6, 72.1. ESI-MS (m/z) cal. for C₈H₅ClNO [M−H]+: 166.01; found 166.06.

### 6-chloro-5-((3-(trifluoromethyl)benzyl)oxy)-1H-indole (5d)

6-chloro-5-((3-(trifluoromethyl)benzyl)oxy)-1H-indole was synthesized in a similar manner as described for compound 5a. White solid, 61.6% yield. ¹H NMR (600 MHz, Chloroform-d): δ 8.06 (s, 1H), 7.79 (s, 1H), 7.72 (d, J=7.7 Hz, 1H), 7.58 (d, J=7.8 Hz, 1H), 7.51 (t, J=7.7 Hz, 1H), 7.46-7.42 (m, 1H), 7.20 (s, 1H), 7.19-7.17 (m, 1H), 6.46 (ddd, J=3.0, 2.0, 0.9 Hz, 1H), 5.19 (s, 2H). ¹³C NMR (151 MHz, Chloroform-d): δ 148.4, 138.3, 131.2, 131.1, 130.9 (q, ²J_{CF}=32.3 Hz), 130.48-130.42 (m, 1H), 129.0, 126.9, 125.4, 124.62 (q, ³J_{CF}=3.8 Hz), 124.1 (q, ¹J_{CF}=272.4 Hz), 123.91 (q, ³J_{CF}=3.8 Hz), 121.4, 119.5, 112.5, 105.8, 102.6, 71.2. ESI-MS (m/z) cal. for C₁₆H₁₀ClF₃NO [M−H]+: 324.04; found 324.12.

### 5-(naphthalen-2-ylmethoxy)-1H-indole (14a)

5-(naphthalen-2-ylmethoxy)-1H-indole was synthesized in a similar manner as described for compound 5a. ¹H NMR (600 MHz, Chloroform-d): δ 8.02 (s, 1H), 7.92 (s, 1H), 7.88-7.81 (m, 3H), 7.58 (dd, J=8.4, 1.6 Hz, 1H), 7.50-7.43 (m, 2H), 7.28 (d, J=8.8 Hz, 1H), 7.23 (d, J=2.4 Hz, 1H), 7.16 (t, J=2.8 Hz, 1H), 6.98 (dd, J=8.8, 2.4 Hz, 1H), 6.49-6.45 (m, 1H), 5.27 (s, 2H). ¹³C NMR (151 MHz, Chloroform-d): δ 153.4, 135.3, 133.3, 133.0, 131.2, 128.3, 128.2, 127.9, 127.7, 126.2, 126.1, 125.9, 125.4, 124.9, 113.1, 111.7, 104.1, 102.5, 71.1. ESI-MS (m/z) cal. for C₁₉H₁₄NO [M−H]+: 272.11; found 272.16.

### 1-(5-(benzyloxy)-6-chloro-1H-indol-3-yl)-2-chloroethan-1-one (1)

Compound 5a (50 mg, 0.19 mmol) was added in a single portion to a stirring solution of 2-Chloro-N,N-dimethylacetamide (598 μL, 5.82 mmol) under argon at 0 ° C The reaction mixture was stirred 10 minutes. Then POCl₃ (180.8 μL, 1.94 mmol) was added dropwise. The reaction mixture was stirred for 2 hours at room temperature. The reaction progress was monitored by TLC. At the end of the reaction, ice and water were added to quench the reaction. The mixture was filtered to get solid. The solid was further purified by silica gel column chromatography (EtOAc:hexane=1:1) to obtain compound 1 (52.6 mg, 81.1%) as a white solid. ¹H NMR (600 MHz, DMSO-d₆): δ 12.11 (s, 1H), 8.42 (d, J=2.8 Hz, 1H), 7.91 (s, 1H), 7.61 (s, 1H), 7.53 (m, 2H), 7.42 (t, J=7.4 Hz, 2H), 7.34 (t, J=7.4 Hz, 1H), 5.23 (s, 2H), 4.86 (s, 2H). ¹³C NMR (151 MHz, DMSO-d₆): δ 186.1, 149.7, 136.9, 135.5, 131.1, 128.4, 127.8, 127.4, 124.9, 118.7, 113.5, 113.4, 105.1, 70.3, 46.3. HRMS (ESI): Cal. for C₁₇H₁₂C₁₂NO₂ [M−H]−: 332.0245, found: 332.0249. HPLC purity at 254 nm, 99.7%.

### 2-chloro-1-(6-chloro-5-(naphthalen-2-ylmethoxy)-1H-indol-3-yl)ethan-1-one (2)

Compound 2 was synthesized in a similar manner as described for compound 1. White solid, 84.5% yield. ¹H NMR (600 MHz, DMSO-d₆) δ 12.12 (d, J=3.2 Hz, 1H), 8.42 (d, J=3.2 Hz, 1H), 8.06 (s, 1H), 8.00-7.92 (m, 4H), 7.67 (dd, J=8.5, 1.7 Hz, 1H), 7.63 (s, 1H), 7.58-7.50 (m, 2H), 5.41 (s, 2H), 4.86 (s, 2H).¹³C NMR (151 MHz, DMSO-d₆) δ 186.67, 150.25, 135.99, 134.99, 133.23, 133.04, 131.61, 128.58, 128.28, 128.11, 126.86, 126.65, 126.55, 125.97, 125.32, 119.25, 114.08, 113.87, 105.76, 71.08, 46.76. HR-MS (ESI) m/z: calc. for C₂₁H₁₄C₁₂NO₂ [M−H]⁻: 382.0402, found: 382.0404. HPLC purity at 254 nm, 98.8%.

### 2-chloro-1-(6-chloro-5-(naphthalen-1-ylmethoxy)-1H-indol-3-yl)ethan-1-one (3)

Compound 3 was synthesized in a similar manner as described for compound 1. White solid, 87.3% yield. ¹H NMR (600 MHz, Methanol-d4) δ 8.19 (d, J=8.3 Hz, 1H), 8.14 (d, J=11.1 Hz, 2H), 7.91 (d, J=8.0 Hz, 1H), 7.87 (d, J=8.3 Hz, 1H), 7.77 (d, J=6.9 Hz, 1H), 7.60-7.47 (m, 4H), 5.66 (s, 2H), 4.66 (s, 2H). ¹H NMR (600 MHz, DMSO-d₆): δ 12.17 12.08 (m, 1H), 8.43 (d, J=3.2 Hz, 1H), 8.17 (d, J=8.2 Hz, 1H), 8.07 (s, 1H), 8.02-7.94 (m, 2H), 7.75 (d, J=6.8 Hz, 1H), 7.61 (s, 1H), 7.61-7.53 (m, 3H), 5.67 (s, 2H), 4.87 (s, 2H). ¹³C NMR (151 MHz, DMSO-d₆): δ 186.1, 149.7, 135.4, 133.2, 132.2, 131.03, 131.02, 128.7, 128.4, 126.5, 126.3, 125.9, 125.3, 124.8, 123.9, 118.6, 113.5, 113.4, 105.1, 69.0, 46.2. HRMS (ESI): Cal. for C₂₁H₁₄C₁₂NO₂ [M−H]⁻: 382.0402, found: 382.0405. HPLC purity at 254 nm, 96.7%.

### 2-chloro-1-(6-chloro-5-((3-(trifluoromethyl)benzyl)oxy)-1H-indol-3-yl)ethan-1-one (22)

Compound 22 was synthesized in a similar manner as described for compound 1. White solid, 81.4% yield. HPLC purity=100%; t_{R}=15.3 min. ¹H NMR (600 MHz, DMSO-d₆) δ 12.14 (d, J=3.2 Hz, 1H), 8.44 (d, J=3.2 Hz, 1H), 7.93 (m, 2H), 7.86 (d, J=7.6 Hz, 1H), 7.72 (d, J=7.8 Hz, 1H), 7.68 (m, 1H), 7.64 (s, 1H), 5.35 (s, 2H), 4.87 (s, 2H). ¹³C NMR (151 MHz, DMSO-d₆) δ 186.63, 150.01, 139.02, 136.09, 131.77, 131.69, 130.04, 129.64 (q, ²J_{CF}=31.6 Hz), 125.35, 124.97 (q, ³J_{CF}=3.9 Hz), 124.70 (q, ¹J_{CF}=272.3 Hz), 124.17 (q, ³J_{CF}=3.9 Hz), 119.12, 114.09, 113.92, 105.67, 70.01, 46.77. HR-MS (ESI) m/z: calc. for C₁₈H₁₁Cl₂F₃NO₂ [M−H]⁻: 400.0119, found: 400.0123. HPLC purity at 254 nm, 99.4%.

### 2-chloro-1-(6-chloro-5-((4-(trifluoromethyl)benzyl)oxy)-1H-indol-3-yl)ethan-1-one (23)

Compound 23 was synthesized using crude compound 5e in a similar manner as described for compound 1. White solid, 78.3% yield. ¹H NMR (600 MHz, DMSO-d6) δ 12.13 (d, J=3.2 Hz, 1H), 8.43 (d, J=3.2 Hz, 1H), 7.91 (s, 1H), 7.80 (d, J=8.2 Hz, 2H), 7.76 (d, J=8.2 Hz, 2H), 7.63 (s, 1H), 5.35 (s, 2H), 4.86 (s, 2H). ¹³C NMR (151 MHz, DMSO) δ 186.05, 149.39, 141.77, 135.50, 131.08, 128.18 (q, ²J_{CF}=31.6 Hz), 127.60, 125.26(q, ³J_{CF}=3.7 Hz), 124.76, 124.36 (q, ¹J_{CF}=271.5 Hz), 118.50, 113.53, 113.33, 104.95, 69.36, 46.21. HR-MS (ESI) m/z: calc. for C₁₈H₁₁Cl₂F₃NO₂ [M−H]⁻: 400.0119, found: 400.0120. HPLC purity at 254 nm, 99.8%.

### 2-chloro-1-(6-chloro-5-((3-fluoro-4-(trifluoromethyl)benzyl)oxy)-1H-indol-3-yl)ethan-1-one (24)

Compound 24 was synthesized using crude compound 5f in a similar manner as described for compound 1. White solid, 76.9% yield. ¹H NMR (600 MHz, DMSO-d₆) δ 12.14 (s, 1H), 8.44 (s, 1H), 7.90 (s, 1H), 7.87 (t, J=7.9 Hz, 1H), 7.67-7.63 (m, 2H), 7.59 (d, J=8.1 Hz, 1H), 5.36 (s, 2H), 4.86 (s, 2H). ¹³C NMR (151 MHz, DMSO-d₆) δ 186.1, 159.6-157.9 (dq, ¹J_{CF}=253.2 Hz, ³J_{CF}=3.7 Hz), 149.2, 145.4 (d, ³J_{CF}- 7.9 Hz), 135.6, 131.2, 127.5 (q, ³J_{CF}=4.1 Hz), 124.8, 123.1 (d, ³J_{CF}=3.3 Hz), 122.6 (q, ³J_{CF}=271.9 Hz), 118.5, 115.7-115.4 (m), 115.1 (d, ²J_{CF}- 21.2 Hz), 113.6, 113.4, 105.0, 68.7, 46.2. HR-MS (ESI) m/z: calc. for C₁₈H₁₀Cl₂F₄NO₂ [M−H]⁻: 418.0025, found: 418.0028. HPLC purity at 254 nm, 98.7%.

### 2-chloro-1-(5-(naphthalen-2-ylmethoxy)-1H-indol-3-yl)ethan-1-one (7)

Compound 7 was synthesized in a similar manner as described for compound 1. White solid, 87.1% yield. ¹H NMR (600 MHz, DMSO-d₆) δ 12.07 (s, 1H), 8.36 (d, J=3.3 Hz, 1H), 8.01 (s, 1H), 7.97-7.91 (m, 3H), 7.81 (d, J=2.5 Hz, 1H), 7.62 (dd, J=8.5, 1.7 Hz, 1H), 7.52 (m, 2H), 7.43 (d, J=8.8 Hz, 1H), 7.02 (dd, J=8.8, 2.5 Hz, 1H), 5.31 (s, 2H), 4.82 (s, 2H). ¹³C NMR (151 MHz, DMSO-d₆) δ 186.57, 155.17, 135.55, 135.43, 133.27, 132.97, 132.09, 128.49, 128.24, 128.07, 126.78, 126.66, 126.52, 126.14, 114.09, 113.89, 113.64, 105.15, 70.26, 46.75. HR-MS (ESI) m/z: calc. for C₁₈H₁₁C₁₂F₃NO₂ [M−H]⁻: 348.0791, found: 348.0792. HPLC purity at 254 nm, 95.8%.

### 1-(5-(benzyloxy)-1H-indol-3-yl)-2-chloroethan-1-one (8)

Compound 8 was synthesized in a similar manner as described for compound 1. White solid, 65.9% yield. ¹H NMR (600 MHz, DMSO-d₆) δ 12.04 (s, 1H), 8.37 (d, J=3.3 Hz, 1H), 7.77 (d, J=2.5 Hz, 1H), 7.49 (d, J=7.5 Hz, 2H), 7.43-7.37 (m, 3H), 7.32 (t, J=7.3 Hz, 1H), 6.96 (dd, J=8.8, 2.5 Hz, 1H), 5.13 (s, 2H), 4.84 (s, 2H). ¹³C NMR (151 MHz, DMSO-d₆) δ 186.5, 155.2, 137.9, 135.4, 132.1, 128.9, 128.2, 128.1, 126.7, 114.0, 113.9, 113.6, 105.0, 70.1, 46.8. HR-MS (ESI) m/z: calc. for C₁₇H₁₄ClNO₂ [M−H]⁻: 298.0635, found: 298.0637. HPLC purity at 254 nm, 99.1%.

### 1-(benzyloxy)-2-methoxy-4-nitrobenzene (7a)

To a solution of compound 6a (200 mg, 1.18 mmol) in N,N-dimethylformamide (6 ml) was added benzyl bromide (181.1 μL, 1.54 mmol), cesium carbonate (771.13 mg, 2.36 mmol). The resultant reaction mixture was stirred at 25 ° C for 5 hours. The reaction was monitored by TLC. On completion of reaction, the reaction mixture was quenched with water and extracted with ethyl acetate (3×30 mL). The combined organic layers were washed with brine, dried over anhydrous Na₂SO₄ and concentrated under reduced pressure. The residue was further purified by silica gel column chromatography (EtOAc:hexane=1:5) to obtain compound 7a (260.0 mg, 85.1%) as a white solid. Alternatively, the residue was further purified by silica gel column chromatography (EtOAc:hexane=1:2) to obtain compound 7a (38.8 mg, 35.1%) as a white solid. ¹H NMR (600 MHz, Chloroform-d) δ 7.84 (dd, J=8.9, 2.6 Hz, 1H), 7.76 (d, J=2.6 Hz, 1H), 7.45-7.42 (m, 2H), 7.42-7.37 (m, 2H), 7.37-7.31 (m, 1H), 6.92 (d, J=8.9 Hz, 1H), 5.24 (s, 2H), 3.96 (s, 3H). ¹³C NMR (151 MHz, Chloroform-d) δ 153.62, 149.37, 141.65, 135.61, 128.82, 128.42, 127.28, 117.60, 111.83, 106.79, 71.18, 56.36. ESI-MS (m/z) cal. for C₁₄H₁₄NO₄ μM+H]+: 260.09; found 260.16.

### 6-methoxy-1H-indol-5-ol (9a)

6-methoxy-1H-indol-5-ol was synthesized using crude compound 8a in a similar manner as described for compound 4a. White solid, 13% yield (2 steps). ¹H NMR (600 MHz, Methanol-d4): δ 7.03 (d, J=3.1 Hz, 1H), 6.94 (s, 1H), 6.92 (s, 1H), 6.24-6.21 (m, 1H), 3.86 (s, 3H). ¹³C NMR (151 MHz, Methanol-d4): δ 146.7, 142.1, 131.9, 124.1, 122.8, 105.6, 101.6, 95.4, 56.6. ESI-MS (m/z) cal. for C₉H₈NO₂ [M−H]+: 162.06; found 162.01.

### 5-(benzyloxy)-6-methoxy-1H-indole (10a)

5-(benzyloxy)-6-methoxy-1H-indole was synthesized in a similar manner as described for compound 5a. White solid, 68.8% yield. ¹H NMR (600 MHz, Chloroform-d): δ 8.01 (s, 1H), 7.51-7.45 (m, 2H), 7.35 (t, J=7.4 Hz, 2H), 7.28 (t, J=7.4 Hz, 1H), 7.12 (s, 1H), 7.04-7.02 (m, 1H), 6.88 (s, 1H), 6.38 (m, 1H), 5.16 (s, 2H), 3.88 (s, 3H). ¹³C NMR (151 MHz, Chloroform-d): δ 147.8, 144.2, 137.8, 130.7, 128.4, 127.6, 127.4, 122.8, 120.6, 105.9, 102.3, 94.9, 72.0, 56.4. ESI-MS (m/z) cal. for C₁₆H₁₄NO₂ [M−H]+: 252.10; found 252.19.

### 6-methoxy-5-(naphthalen-1-ylmethoxy)-1H-indole (10b)

6-methoxy-5-(naphthalen-1-ylmethoxy)-1H-indole was synthesized by a procedure used previously for compound 5a. White solid, 63.6% yield. ¹H NMR (600 MHz, Chloroform-d): δ 8.20 (d, J=8.3 Hz, 1H), 8.00 (s, 1H), 7.88 (d, J=7.8 Hz, 1H), 7.81 (d, J=8.2 Hz, 1H), 7.64 (d, J=6.9 Hz, 1H), 7.57-7.48 (m, 2H), 7.46-7.41 (m, 1H), 7.21 (s, 1H), 7.05-7.02 (m, 1H), 6.91 (s, 1H), 6.39 (m, 1H), 5.59 (s, 2H), 3.88 (s, 3H). ¹³C NMR (151 MHz, Chloroform-d): δ 148.1, 144.3, 133.7, 133.1, 131.5, 130.9, 128.6, 128.5, 126.2, 126.2, 125.7, 125.4, 123.8, 122.9, 120.7, 106.6, 102.4, 95.1, 70.8, 56.4. ESI-MS (m/z) cal. for C₂₀H₁₆NO₂ [M−H]+: 302.12; found 302.17.

### 2-chloro-1-(6-methoxy-5-(naphthalen-2-ylmethoxy)-1H-indol-3-yl)ethan-1-one (5)

5 was synthesized in a similar manner as described for 1. White solid, 89.4% yield. ¹H NMR (600 MHz, DMSO-d₆) δ 8.09 (s, 1H), 7.98 (s, 1H), 7.95-7.80 (m, 4H), 7.65 (dd, J=8.4, 1.7 Hz, 1H), 7.53-7.47 (m, 2H), 7.08 (s, 1H), 5.32 (s, 2H), 4.68 (s, 2H), 3.91 (s, 3H). ¹³C NMR (151 MHz, DMSO-d₆) δ 186.27, 147.84, 145.24, 134.46, 132.57, 132.34, 131.78, 131.01, 127.34, 127.13, 126.88, 125.70, 125.47, 125.30, 125.03, 118.03, 113.42, 105.58, 95.04, 70.64, 54.99, 44.95. ¹H NMR (600 MHz, Methanol-d4) δ 8.09 (s, 1H), 7.98 (s, 1H), 7.95-7.80 (m, 4H), 7.65 (dd, J=8.4, 1.4 Hz, 1H), 7.53-7.47 (m, 2H), 7.08 (s, 1H), 5.32 (s, 2H), 4.68 (s, 2H), 3.91 (s, 3H). ¹³C NMR (151 MHz, Methanol-d4) δ 188.0, 149.6, 147.0, 136.2, 134.4, 134.1, 133.6, 132.8, 129.1, 128.9, 128.7, 127.5, 127.3, 127.1, 126.8, 119.8, 115.2, 107.4, 96.8, 72.4, 56.8, 46.7. HR-MS (ESI) m/z: calc. for C₂₂H₁₉ClNO₃ [M−H]⁻: 378.0897, found: 378.0898. HPLC purity at 254 nm, 98.2%.

### 1-(5-(benzyloxy)-6-methoxy-1H-indol-3-yl)-2-chloroethan-1-one (4)

4 was synthesized in a similar manner as described for 1. White solid, 90.0% yield. ¹H NMR (600 MHz, Methanol-d4, Chloroform-d₄) δ 7.93 (s, 1H), 7.89 (s, 1H), 7.53-7.51 (m, 2H), 7.38 (dd, J=8.4, 6.8 Hz, 2H), 7.34-7.28 (m, 1H), 7.02 (s, 1H), 5.21 (s, 2H), 4.59 (s, 2H), 3.93 (s, 3H). ¹³C NMR (151 MHz, Methanol-d4) δ 187.84, 148.86, 146.54, 137.59, 132.36, 132.04, 128.80, 128.19, 128.09, 119.29, 114.76, 106.61, 96.00, 71.98, 56.47, 45.97.

HR-MS (ESI) m/z: calc. for C₁₈H₁₇ClNO₃ [M−H]⁻: 328.0740, found: 328.0742. HPLC purity at 254 nm, 98.3%.

### 2-chloro-1-(6-methoxy-5-(naphthalen-1-ylmethoxy)-1H-indol-3-yl)ethan-1-one (6)

6 was synthesized using crude compound 10c in a similar manner as described for compound 1. White solid, 81.1% yield. ¹H NMR (600 MHz, Methanol-d4) δ 8.22 (d, J=8.3 Hz, 1H), 8.02 (d, J=2.6 Hz, 2H), 7.90 (dd, J=8.0, 1.5 Hz, 1H), 7.86 (d, J=8.2 Hz, 1H), 7.68 (d, J=7.0 Hz, 1H), 7.54 (m, 2H), 7.47 (dd, J=8.3, 7.0 Hz, 1H), 7.06 (s, 1H), 5.61 (s, 2H), 4.64 (s, 2H), 3.89 (s, 3H). ¹H NMR (600 MHz, DMSO-d₆) δ 11.95-11.88 (m, 1H), 8.27 (d, J=3.0 Hz, 1H), 8.17 (d, J=8.2 Hz, 1H), 8.01-7.98 (m, 1H), 7.95 (d, J=8.2 Hz, 1H), 7.88 (s, 1H), 7.69 (d, J=6.7 Hz, 1H), 7.62-7.56 (m, 2H), 7.53 (dd, J=8.2, 7.0 Hz, 1H), 7.06 (s, 1H), 5.53 (s, 2H), 4.83 (s, 2H), 3.79 (s, 3H). ¹³C NMR (151 MHz, DMSO-d₆) δ 185.9, 147.8, 145.4, 133.2, 132.9, 132.8, 131.3, 128.6, 128.3, 126.8, 126.3, 125.9, 125.3, 124.0, 118.2, 113.7, 105.5, 95.7, 69.1, 55.6, 46.1. HR-MS (ESI) m/z: calc. for C₂₂H₁₉ClNO₃ [M−H]⁻: 378.0897, zfound: 378.0899. HPLC purity at 254 nm, 96.8%.

### 5-(benzyloxy)-6-chloro-1H-indole-3-carbaldehyde (15a)

Phosphorus oxychloride (0.25 mL, 2.66 mmol) was added dropwise to a stirred mixture of N,N-dimethylformamide (1.03 mL, 13.32 mmol) and indole 5a (566.98 mg, 2.2 mmol) at 0 ° C The resulting mixture was stirred at 0 ° C for 0.5 h then at 40 ° C for 2 h. Ice was added, followed by a solution of sodium hydroxide (2 M), and the mixture was heated under reflux for 1 min. On cooling, the solution was extracted with ethyl acetate (3×20 mL). The combined organic phases were washed with brine, dried over Na₂SO₄, and concentrated. Column chromatography with hexane:ethyl acetate (1:1) gave a white solid (409.22 mg, 65.1%). ¹H NMR (600 MHz, Chloroform-d) δ 11.24 (s, 1H), 9.96 (d, J=1.4 Hz, 1H), 7.94 (d, J=1.4 Hz, 1H), 7.79 (dd, J=3.1, 1.6 Hz, 1H), 7.57-7.51 (m, 3H), 7.43-7.37 (m, 2H), 7.32 (m, 1H), 5.21 (s, 2H). ¹³C NMR (600 MHz, Chloroform-d, DMSO-d6) δ 184.99, 150.69, 137.01, 136.89, 131.90, 128.48, 127.83, 127.31, 123.80, 120.64, 118.80, 113.54, 105.61, 71.27. ¹H NMR (600 MHz, DMSO-d₆) δ 12.09 (s, 1H), 9.91 (s, 1H), 8.27 (s, 1H), 7.83 (s, 1H), 7.62 (s, 1H), 7.53 (d, J=7.4 Hz, 2H), 7.41 (t, J=7.6 Hz, 2H), 7.34 (t, J=7.3 Hz, 1H), 5.22 (s, 2H). ¹³C NMR (151 MHz, DMSO-d₆) δ 184.9, 149.8, 139.0, 136.9, 131.4, 128.4, 127.8, 127.4, 123.5, 118.8, 117.9, 113.6, 104.7, 70.3. ESI-MS (m/z) cal. for C₁₆H₁₁ClNO₂ [M−H]+: 284.05; found 284.12.

### 6-chloro-5-(naphthalen-1-ylmethoxy)-1H-indole-3-carbaldehyde (15b)

6-chloro-5-(naphthalen-1-ylmethoxy)-1H-indole-3-carbaldehyde was prepared in a similar manner as described for compound 15a. White solid, 72.4% yield. ¹H NMR (600 MHz, DMSO-d₆) δ 12.10 (s, 1H), 9.95 (s, 1H), 8.30 (s, 1H), 8.17 (d, J=8.2 Hz, 1H), 8.02 (s, 1H), 7.99 (d, J=7.7 Hz, 1H), 7.96 (d, J=8.2 Hz, 1H), 7.76 (d, J=6.9 Hz, 1H), 7.66-7.52 (m, 4H), 5.67 (s, 2H). ESI-MS (m/z) cal. for C₂₀H₁₃ClNO₂ [M−H]+: 334.06; found 334.01.

### 6-chloro-5-(naphthalen-2-ylmethoxy)-1H-indole-3-carbaldehyde (15c)

6-chloro-5-(naphthalen-2-ylmethoxy)-1H-indole-3-carbaldehyde was prepared in a similar manner as described for compound 15a. White solid, 58.9% yield. ¹H NMR (600 MHz, Methanol-d4): δ 9.86 (s, 1H), 8.08 (s, 1H), 8.01 (s, 1H), 7.97 (s, 1H), 7.90 (d, J=8.5 Hz, 1H), 7.88-7.85 (m, 2H), 7.66 (dd, J=8.4, 1.6 Hz, 1H), 7.56 (s, 1H), 7.48 (qd, J=7.1, 3.4 Hz, 2H), 5.37 (s, 2H). ¹³C NMR (151 MHz, Methanol-d4): δ 187.3, 152.2, 140.1, 136.0, 134.8, 134.6, 133.5, 129.2, 129.0, 128.7, 127.3, 127.2, 127.1, 126.4, 125.1, 121.9, 119.9, 114.6, 106.8, 72.6, 49.6, 49.4, 49.3, 49.1, 49.0, 48.9, 48.7, 48.6. ESI-MS (m/z) cal. for C₂₀H₁₃ClNO₂ [M−H]+: 334.06; found 334.09.

### N-((5-(benzyloxy)-6-chloro-1H-indol-3-yl)methyl)acrylamide (11)

Compound 15a (214.30 mg, 0.75 mmol) was stirred together with hydroxylamine hydrochloride (130.64 mg, 1.88 mmol) and pyridine (4.5 mL) until the aldehyde disappeared from the reaction mixture (TLC). After quenched with water, the mixture was acidified with 10% HCl and extracted with Et₂O. The combined organic extracts were washed successively with 10% HCl and water, dried over Na₂SO₄, and concentrated to get crude product 21 (ESI-MS: m/z 301.01 μM+H]+). A suspension of 16a (198.49 mg, 0.66 mol) in glacial acetic acid (10 mL) was treated with zinc dust (431.5 mg, 6.6 mmol) and heated to 50 ° C for 10 h. The supernatant was decanted into ice-cold 2 μM NaOH (40 mL) and extracted with several portions of Et₂O. The organic extracts were dried (MgSO4) and concentrated under reduced pressure to get crude product 24 (ESI-MS: m/z 287.11 μM+H]+). To a solution of 17a (143.38 mg, 0.5 mmol) in DCM (10 mL) at 0 ° C was added Et₃N (101.19 mg, 1 mmol) and acryloyl chloride (54.31 mg, 0.6 mmol). Then the solution was stirred at room temperature. After the reaction was complete (monitored by TLC), water (20 mL) was added. The reaction mixture was extracted with DCM and washed with brine, dried over Na₂SO₄, filtered and then concentrated. Column chromatography with hexane:ethyl acetate (1:1) gave 11 as a white solid (85.88 mg, 33.6%). ¹H NMR (600 MHz, DMSO-d₆) δ 10.91 (s, 1H), 8.38 (t, J=5.7 Hz, 1H), 7.51 (d, J=7.4 Hz, 2H), 7.44 (s, 1H), 7.42-7.37 (m, 3H), 7.34 (t, J=7.4 Hz, 1H), 7.28 (d, J=2.6 Hz, 1H), 6.26 (dd, J=17.1, 10.1 Hz, 1H), 6.15 (dd, J=17.1, 2.4 Hz, 1H), 5.60 (dd, J=10.0, 2.4 Hz, 1H), 5.13 (s, 2H), 4.45 (d, J=5.6 Hz, 2H). ¹³C NMR (151 MHz, DMSO-d₆) δ 164.4, 147.1, 137.1, 131.9, 131.1, 128.4, 127.8, 127.6, 125.7, 125.4, 125.0, 117.2, 112.4, 112.2, 103.6, 70.6, 45.6. HR-MS (ESI) m/z: calc. for C₁₉H₁₈ClN₂O₂ μM−H]⁻: 339.0900, found: 339.0902. HPLC purity at 254 nm, 100.0%.

Compounds 9 and 10 were prepared in a similar manner as described for compound 11.

### N-((6-chloro-5-(naphthalen-2-ylmethoxy)-1H-indol-3-yl)methyl)acrylamide (9)

White solid, 31.5% yield. ¹H NMR (600 MHz, CDCl₃) δ 7.99 (s, 1H), 7.92-7.85 (m, 3H), 7.66 (dd, J=8.4, 1.7 Hz, 1H), 7.53-7.46 (m, 2H), 7.43 (s, 1H), 7.38 (s, 1H), 7.23 (s, 1H), 6.26 (dd, J=17.1, 2.3 Hz, 1H), 6.20 (dd, J=17.1, 9.9 Hz, 1H), 5.63 (dd, J=9.9, 2.3 Hz, 1H), 5.32 (s, 2H), 4.58 (s, 2H). ¹³C NMR (151 MHz, Chloroform-d) δ 165.3, 148.7, 134.5, 133.3, 133.0, 131.2, 130.6, 128.3, 128.0, 127.7, 126.5, 126.2, 126.1, 125.9, 125.6, 125.3, 124.2, 120.2, 112.8, 112.7, 103.7, 71.7, 35.0. HR-MS (ESI) m/z: calc. for C₂₃H₁₈ClN₂O₂ [M−H]⁻: 389.1057, found: 389.1060. HPLC purity at 254 nm, 97.2%.

### N-((6-chloro-5-(naphthalen-1-ylmethoxy)-1H-indol-3-yl)methyl)acrylamide (10)

White solid, 36.1% yield. ¹H NMR (600 MHz, DMSO-d₆) δ 10.92 (s, 1H), 8.42 (t, J=5.6 Hz, 1H), 8.18 (d, J=8.4 Hz, 1H), 7.99 (d, J=7.7 Hz, 1H), 7.95 (d, J=8.2 Hz, 1H), 7.78 (d, J=6.9 Hz, 1H), 7.64-7.51 (m, 4H), 7.44 (s, 1H), 7.30 (d, J=2.4 Hz, 1H), 6.28 (dd, J=17.1, 10.1 Hz, 1H), 6.15 (dd, J=17.1, 2.3 Hz, 1H), 5.59 (dd, J=10.1, 2.3 Hz, 1H), 5.58 (s, 2H), 4.50 (d, J=5.5 Hz, 2H). ¹³C NMR (151 MHz, DMSO-d₆) δ 164.4, 147.2, 133.3, 132.5, 131.9, 131.1, 131.1, 128.6, 128.5, 126.5, 126.4, 126.0, 125.7, 125.4, 125.3, 125.0, 123.9, 117.1, 112.5, 112.4, 103.6, 69.2, 45.6. HR-MS (ESI) m/z: calc. for C₂₄H₂₁N₂O₃ [M−H]⁻: 389.1057, found: 389.1060. HPLC purity at 254 nm, 98.7%.

### N-((5-(benzyloxy)-6-chloro-1H-indol-3-yl)methyl)-2-chloroacetamide (12)

To a solution of crude compound 17a (143.38 mg, 0.5 mmol) in DCM (5 mL) at 0° C., Et₃N (101.19 mg, 1.0 mmol) and chloroacetyl chloride (67.76 mg, 0.6 mmol) were added.

Then the solution was stirred at room temperature. After the reaction was complete (monitored by TLC), water (10 mL) was added. The reaction mixture was extracted with DCM and washed with brine, dried over Na₂SO₄, filtered and then concentrated. The residue was further purified by silica gel column chromatography (EtOAc:hexane=1:2) to obtain compound 12 (132.58 mg, 33.1%) as a white solid. ¹H NMR (600 MHz, DMSO-d₆) δ 10.92 (s, 1H), 8.53 (t, J=5.6 Hz, 1H), 7.55-7.50 (m, 2H), 7.46-7.39 (m, 4H), 7.37-7.31 (m, 1H), 7.28 (d, J=2.2 Hz, 1H), 5.14 (s, 2H), 4.41 (d, J=5.5 Hz, 2H), 4.07 (s, 2H). ¹³C NMR (600 MHz, DMSO-d₆) δ 166.11, 147.64, 137.62, 131.58, 128.88, 128.26, 128.02, 126.05, 125.95, 117.65, 112.92, 112.35, 103.98, 71.08, 43.19, 34.81. HR-MS (ESI) m/z: calc. for C₁₈H₁₅C₁₂N₂O₂ [M−H]⁻: 361.0511, found: 361.0514. HPLC purity at 254 nm, 100.0%.

Compounds 13, 14 and 15 were prepared in a similar manner as described for compound 12.

### 2-chloro-N-((6-chloro-5-(naphthalen-1-ylmethoxy)-1H-indol-3-yl)methyl)acetamide (14)

White solid, 35.4% yield. ¹H NMR (600 MHz, DMSO-d₆) δ 10.93-10.89 (m, 1H), 8.61 (t, J=5.6 Hz, 1H), 8.15 (d, J=8.1 Hz, 1H), 8.02-7.91 (m, 2H), 7.76 (d, J=6.9 Hz, 1H), 7.64-7.51 (m, 4H), 7.46 (s, 1H), 7.29 (d, J=2.4 Hz, 1H). 5.57 (s, 2H), 4.47-4.42 (m, 2H), 4.06 (s, 2H). ¹³C NMR (151 MHz, DMSO-d₆) δ 166.4, 147.7, 133.7, 132.9, 131.6, 131.6, 129.2, 128.9, 127.1, 126.9, 126.5, 126.0, 125.9, 125.9, 124.4, 117.6, 113.0, 112.4, 103.9, 69.7, 43.2, 34.8. HR-MS (ESI) m/z: calc. for C₂₂H₁₇C₁₂N₂O₂ [M−H]⁻: 411.0667, found: 411.0670. HPLC purity at 254 nm, 100.0%.

### 2-chloro-N-((6-chloro-5-(naphthalen-2-ylmethoxy)-1H-indol-3-yl)methyl)acetamide (13)

White solid, 38.8% yield. ¹H NMR (600 MHz, DMSO-d₆) δ 10.92 (d, J=2.5 Hz, 1H), 8.52 (t, J=5.5 Hz, 1H), 8.05 (s, 1H), 7.99-7.91 (m, 3H), 7.66 (dd, J=8.4, 1.7 Hz, 1H), 7.58-7.51 (m, 2H), 7.46 (d, J=4.5 Hz, 2H), 7.29 (d, J=2.5 Hz, 1H), 5.32 (s, 2H), 4.42 (d, J=5.5 Hz, 2H), 4.05 (s, 2H). ¹³C NMR (151 MHz, DMSO-d6) δ 166.0, 147.4, 134.9, 132.9, 132.7, 131.3, 128.2, 128.0, 127.8, 126.5, 126.5, 126.3, 125.9, 125.7, 125.7, 117.4, 112.7, 112.1, 103.9, 71.0, 42.8, 34.5. HR-MS (ESI) m/z: calc. for C₂₂H₁₇C₁₂N₂O₂ [M−H]⁻: 411.0667, found: 411.0669. HPLC purity at 254 nm, 99.3%.

### 2-chloro-N-((6-chloro-5-((3-(trifluoromethyl)benzyl)oxy)-1H-indol-3-yl)methyl) acetamide (15)

White solid, 31.1% yield. ¹H NMR (600 MHz, DMSO-d₆) δ 10.95 (d, J=2.5 Hz, 1H), 8.53 (t, J=5.6 Hz, 1H), 7.89 (s, 1H), 7.83 (d, J=7.6 Hz, 1H), 7.74-7.65 (m, 2H), 7.46 (s, 1H), 7.42 (s, 1H), 7.30 (d, J=2.5 Hz, 1H), 5.26 (s, 2H), 4.41 (d, J=5.6 Hz, 2H), 4.07 (s, 2H). ¹³C NMR (151 MHz, DMSO-d₆) δ 165.6, 146.9, 138.7, 131.4, 131.2, 129.5, 129.2 (q, ²J_{CF}=31.6 Hz), 125.6, 124.5 (q, ³J_{CF}=3.6 Hz), 124.3 (q, ¹J_{CF}=272.2 Hz), 123.8 (q, ³J_{CF}=3.8 Hz), 121.5, 117.1, 112.5, 111.9, 103.7, 69.8, 42.7, 34.3. HR-MS (ESI) m/z: calc. for C₁₉H₁₄C₁₂F₃N₂O₂[M−H]⁻: 429.0384, found: 429.0386. HPLC purity at 254 nm, 100.0%.

### 6-chloro-5-(quinolin-2-ylmethoxy)-1H-indole-3-carbaldehyde (15e)

6-chloro-5-(quinolin-2-ylmethoxy)-1H-indole-3-carbaldehyde was prepared in a similar manner as described for 15a. White solid, 82.5% yield. ¹H NMR (600 MHz, DMSO-d₆) δ 12.15 (s, 1H), 9.88 (s, 1H), 8.45 (d, J=8.5 Hz, 1H), 8.27 (s, 1H), 8.05 (d, J=8.4 Hz, 1H), 8.00 (d, J=8.0 Hz, 1H), 7.88 (s, 1H), 7.83-7.78 (m, 1H), 7.76 (d, J=8.5 Hz, 1H), 7.69-7.60 (m, 2H), 5.49 (s, 2H). ESI-MS (m/z) cal. for C₁₉H₁₂ClN₂O₂[M−H]+: 335.06; found 335.11.

### 6-chloro-5-((3-(trifluoromethyl)benzyl)oxy)-1H-indole-3-carbaldehyde (15f)

6-chloro-5-((3-(trifluoromethyl)benzyl)oxy)-1H-indole-3-carbaldehyde was prepared in a similar manner as described for 15a. White solid, 72.1% yield. ¹H NMR (600 MHz, DMSO-d₆) δ 12.10 (s, 1H), 9.91 (s, 1H), 8.29 (s, 1H), 7.92 (s, 1H), 7.85 (d, J=8.7 Hz, 2H), 7.72 (d, J=7.8 Hz, 1H), 7.68 (d, J=7.6 Hz, 1H), 7.64 (s, 1H), 5.34 (s, 2H). ESI-MS (m/z) cal. for C₁₉H₁₀ClF₃NO₂ [M−H]+: 352.04; found 352.16.

### (E)-3-(5-(benzyloxy)-6-chloro-1H-indol-3-yl)-2-cyanoacrylamide (16)

To a solution of 15a (57.15 mg, 0.2 mmol) in THF (2 mL) and EtOH (2 ml) was added 2-cyanoacetamide (25.22 mg, 0.3 mmol) and piperidine (10 μL, 0.1 mmol). The reaction was stirred for 48 hours at room temperature. The yellowish precipitate was filtered and the solids were washed with THF and dried in vacuo to afford 39.68 mg (56.4%) of 16 as a yellow solid. ¹H NMR (600 MHz, DMSO-d₆) δ 12.14 (s, 1H), 8.47 (s, 1H), 8.44 (s, 1H), 7.83 (s, 1H), 7.74 (s, 1H), 7.66 (s, 1H), 7.55 (m, 3H), 7.44 (m, 2H), 7.36 (m, 1H), 5.26 (s, 2H). ¹³C NMR (151 MHz, DMSO-d₆) δ 163.9, 149.4, 142.1, 136.9, 131.0, 130.5, 128.5, 127.9, 127.5, 126.8, 118.8, 118.7, 113.8, 109.6, 102.9, 97.8, 70.6. HR-MS (ESI) m/z: calc. for C₁₉H₁₃ClN₃O₂[M−H]⁻: 350.0696, found: 350.0699. HPLC purity at 254 nm, 99.9%.

Compounds 17, 18, 19, 20, and 21 were prepared in a similar manner as described for compound 16.

### (E)-3-(6-chloro-5-(naphthalen-2-ylmethoxy)-1H-indol-3-yl)-2-cyanoacrylamide (17)

Yellow solid, 80.1% yield. ¹H NMR (600 MHz, DMSO-d₆) δ 12.18 (s, 1H), 8.49 (s, 1H), 8.45 (s, 1H), 8.07 (s, 1H), 7.99 (d, J=8.5 Hz, 1H), 7.97-7.92 (m, 2H), 7.90 (s, 1H), 7.75 (s, 1H), 7.70-7.65 (m, 2H), 7.55 (m, 3H), 5.43 (s, 2H). ¹³C NMR (600 MHz, DMSO-d₆) δ 163.9, 149.4, 142.2, 134.4, 132.8, 132.6, 131.0, 130.5, 128.1, 127.8, 127.6, 126.8, 126.4, 126.3, 126.2, 125.7, 118.8, 118.7, 113.8, 109.6, 103.1, 97.8, 70.9. HR-MS (ESI) m/z: calc. for C₂₃H₁₅ClN₃O₂ [M−H]⁻: 400.0853, found: 400.0854. HPLC purity at 254 nm, 100.0%.

### (E)-3-(6-chloro-5-(naphthalen-1-ylmethoxy)-1H-indol-3-yl)-2-cyanoacrylamide (18)

Yellow solid, 67.4% yield. ¹H NMR (600 MHz, DMSO-d₆) δ 12.25 (s, 1H), 8.54 (s, 6H), 8.47 (s, 1H), 8.20-8.15 (m, 1H), 8.05 (s, 6H), 8.01 (dd, J=8.0, 1.5 Hz, 1H), 7.97 (d, J=8.3 Hz, 1H), 7.82 (dd, J=7.0, 1.2 Hz, 1H), 7.74 (s, 1H), 7.67 (s, 1H), 7.64-7.52 (m, 4H), 5.70 (s, 2H). ¹³C NMR (151 MHz, DMSO-d₆) δ 163.9, 149.5, 142.4, 133.3, 132.4, 131.1, 130.5, 129.7, 129.1, 128.7, 128.5, 126.9, 126.4, 126.0, 125.5, 123.9, 118.8, 113.8, 109.7, 107.0, 103.0, 97.8, 69.1. HR-MS (ESI) m/z: calc. for C₂₃H₁₅ClN₃O₂ [M−H]⁻: 400.0853, found: 400.0856. HPLC purity at 254 nm, 98.8%.

### (E)-3-(6-chloro-5-((4,4-difluorocyclohexyl)methoxy)-1H-indol-3-yl)-2-cyanoacrylamide (19)

Yellow solid, 73.9% yield. ¹H NMR (600 MHz, DMSO-d₆) δ 12.18 (s, 1H), 8.45 (s, 1H), 8.42 (s, 1H), 7.75 (s, 1H), 7.67 (s, 1H), 7.62 (s, 1H), 7.51 (s, 1H), 4.02 (d, J=6.1 Hz, 2H), 2.08-1.81 (m, 7H), 1.48-1.36 (m, 2H). ¹³C NMR (151 MHz, DMSO-d₆) δ 164.5, 150.0, 142.7, 131.3, 130.6, 127.1, 124.7 (q, ¹J_{CF}=241.6 Hz), 119.04, 118.99, 114.0, 109.9, 102.6, 97.9, 73.0, 35.2, 32.7 (q, ²J_{CF}=22.7 Hz), 25.7, 25.6. HR-MS (ESI) m/z: calc. for C₁₉H₁₇ClF₂N₃O₂ [M−H]⁻: 392.0977, found: 392.0981. HPLC purity at 254 nm, 96.2%.

### (E)-3-(6-chloro-5-(quinolin-2-ylmethoxy)-1H-indol-3-yl)-2-cyanoacrylamide (20)

Yellow solid, 63.4% yield. ¹H NMR (600 MHz, DMSO-d₆) δ 12.25 (s, 1H), 8.51-8.46 (m, 2H), 8.45 (s, 1H), 8.06-8.00 (m, 2H), 7.93 (s, 1H), 7.85-7.78 (m, 2H), 7.73 (s, 1H), 7.70 (s, 1H), 7.64 (m, 1H), 7.54 (s, 1H), 5.53 (s, 2H). ¹³C NMR (151 MHz, DMSO-d₆) δ 163.8, 157.2, 149.1, 146.8, 142.1, 137.1, 130.9, 130.5, 129.9, 128.4, 127.9, 127.1, 126.7, 126.6, 119.3, 118.6, 113.8, 109.5, 102.9, 97.8, 71.9. HR-MS (ESI) m/z: calc. for C₂₂H₁₄ClN₄O₂[M−H]⁻: 401.0805, found: 401.0807. HPLC purity at 254 nm, 100.0%.

### 3-(6-chloro-5-((3-(trifluoromethyl)benzyl)oxy)-1H-indol-3-yl)-2-cyanoacrylamide (21)

Yellow solid, 61.9% yield. ¹H NMR (600 MHz, DMSO-d₆) δ 12.26 (s, 1H), 8.47 (s, 1H), 8.45 (s, 1H), 7.92 (s, 1H), 7.87-7.82 (m, 2H), 7.76-7.66 (m, 4H), 7.55 (s, 1H), 5.38 (s, 2H). ¹³C NMR (151 MHz, DMSO-d₆) δ 164.26, 149.61, 142.67, 138.91, 131.88, 131.47, 131.04, 130.11, 129.68 (q, ²J_{CF}=31.6 Hz), 127.27, 125.35, 125.07 (q, ³J_{CF}=3.7 Hz), 124.68 (q, ¹J_{CF}=272.4 Hz), 124.26 (q, ³J_{CF}=4.1 Hz), 119.22, 119.14, 114.35, 110.06, 103.53, 98.31, 70.22, 40.54. HR-MS (ESI) m/z: calc. for C₂₀H₁₂ClF₃N₃O₂ [M−H]⁻: 418.0570, found: 418.0574. HPLC purity at 254 nm, 99.1%.

### N-((5-(benzyloxy)-6-chloro-1H-indol-3-yl)methyl)-2-((dimethylamino)methyl) acrylamide (25)

Compound 17a (28.68 mg, 0.1 mmol,), 2-[(dimethylamino)methyl]prop-2-enoicacid (15.50 mg, 0.12 mmol), HATU (45.63 mg, 0.12 mmol), and diisopropylethylamine (25.85 mg, 0.2 mmol) were dissolved in dichloromethane (5 ml) and stirred at 25 ° C for 10 h. After the reaction was complete (monitored by TLC), water (10 mL) was added. The organic layer was extracted with DCM (3×20 mL) and washed with brine, dried over Na₂SO₄, filtered and then concentrated. The residue was further purified by silica gel column chromatography (EtOAc:hexane=1:1) to obtain compound 25 (29.88 mg, 75.1%) as a white solid. ¹H NMR (600 MHz, Chloroform-d) δ 9.15 (s, 1H), 8.58 (s, 1H), 7.52 (d, J=7.1 Hz, 2H), 7.42 (s, 1H), 7.37 (dd, J=8.3, 7.1 Hz, 2H), 7.33-7.28 (m, 1H), 7.22 (s, 1H), 7.09 (d, J=2.3 Hz, 1H), 6.25 (d, J=1.6 Hz, 1H), 5.51 (s, 1H), 5.14 (s, 2H), 4.59 (d, J=5.4 Hz, 2H), 3.15 (s, 2H), 2.17 (s, 6H). ¹H NMR (599 MHz, DMSO-d₆) δ 10.89 (s, 1H), 8.91 (t, J=5.5 Hz, 1H), 7.51 (d, J=7.3 Hz, 2H), 7.44 (s, 1H), 7.43-7.39 (m, 3H), 7.33 (t, J=7.3 Hz, 1H), 7.26 (d, J=2.2 Hz, 1H), 6.01 (m, 1H), 5.75 (s, 1H), 5.13 (s, 2H), 4.49-4.44 (m, 2H). Note: CH₃ was buried in the DMSO-d₆ peak. ¹³C NMR (151 MHz, DMSO-d₆) δ 166.7, 147.6, 137.6, 131.7, 128.9, 128.3, 128.0, 126.0, 125.6, 117.7, 116.0, 113.0, 112.7, 104.0, 71.2, 55.4, 40.9, 34.7. HR-MS (ESI) m/z: calc. for C₂₂H₂₅ClN₃O₂ μM+H]⁺: 398.1635, found: 398.1631. HPLC purity at 254 nm, 97.2%.

### Molecular Modeling.

Commercially available 200000 compounds from the Specs database were downloaded from the website as .mol2 files. A collection of 900 in-house compounds was saved using .sdf file format. All possible tautomers, stereoisomers, and ionization states of each compound were generated at a pH range of 7.0±2.0 using the Schrodinger Suite 2021 Ligand Preparation tool (Schr6dinger, LLC, New York, NY, 2022).⁷² The crystal structure of TEAD2 complexed with YAP peptide was retrieved from the Protein Data Bank (PDB id: 5EMV).⁴⁹ All water molecules were removed. The clean protein procedure was then conducted using the Protein Preparation Wizard module in Schrodinger. Briefly, hydrogen atoms were added to the complex. The module used ProtAssign to optimize hydroxyl, His, Asn, and Gln protonation states automatically. Finally, the complex was submitted to a restrained minimization using the OPLS2005 force field to relieve steric clashes. The bounding box of size 12 Å×12 Å×12 Å for molecular docking was centered on the residue Cys380 in the central pocket. Alternatively, a bounding box of size 24 Å×24 Å×24 Å for molecular docking was centered on the central pocket. Default parameter settings generated by the program Glide for Standard Precision (SP) docking were used.

### Protein Expression and Purification.

Human TEAD2-YBD (217-447) (TEAD2_HUMAN, UniProtKB Q15562) was expressed and purified as previously described.⁷⁹ To eliminate endogenous palmitoylation, Cys380Ser mutant (TEAD2-C380S) was generated and purified using the same protocol. [00392]hTEAD2 was expressed, purified, and deacylated as reported.⁵² Briefly, the yap-binding domain (YBD) of human TEAD4 (217-434) was cloned in pET28a vector with the N terminal 6x His tag. The recombinant protein was induced at 16 ° C using 0.2 mM isopropyl bD-1-thiogalactopyranoside (IPTG) for 10 h for overexpression in Escherichia coli strain BL2 (DE3) CodonPlus competent cells (Invitrogen). Then cells were harvested, followed by lysis and the supernatant was loaded onto the HisTrap FF column (GE Healthcare). The hTEAD4-YBD was eluted with elution buffer (50 mM Hepes 8.0, 300 mM NaCl, 10% Glycerol, 1 mM TCEP, 125 mM imidazole). To eliminate the endogenous fatty acid, the protein was incubated with 0.2 mM hydroxylamine for 2 h and further purified through gel filtration chromatography in the final buffer (25 mM Hepes pH 8.0, 100 mM NaCl, 1 mM TCEP, and 5% glycerol). Collected fractions were concentrated to 5 mg/mL and flash frozen for further use. To prepare fully palmitoylated TEAD4 (Pal-TEAD4), wild-type TEAD4 was incubated with 2 eq. of palmitoyl coenzyme A lithium salt (Sigma-Aldrich #P9716) for 1 h at room temperature followed by an additional gel filtration step.

### Intact Protein Mass Measurement.

The intact mass spectrometry analysis involved a preparatory step where 22 at a concentration of 50 μM was incubated with wild type TEAD2-YBD at 2.5 μM in tris buffer for 20 hours at room temperature, with subsequent sample preparation by adding 5 μL of formic acid to 20 μL of the analyte. This analysis was performed using an Agilent 6220 Time-of-Flight (TOF) mass spectrometer equipped with an electro spray ionization (ESI) source set to positive mode. The instrumental conditions were optimized to a gas temperature of 350 ° C, a drying gas flow of 8.0 L/min using N₂, and a nebulizer pressure of 30 psig. Chromatographic separation was achieved utilizing an Agilent 1100 series system, which included a G13793 degasser, a G1312B binary pump, and a G1367C auto sampler, with a set injection volume of 1 μL. The mobile phase comprised a 50/50 mixture of water and acetonitrile, both containing 0.1% formic acid, delivered at a flow rate of 0.2 mL/min. The LC/MS raw data was processed using BioConfirm (Version B.09.00, Agilent Technologies, Santa Clara, CA, USA) to generate intact protein masses with maximum entropy, range 10-50 kDa, +H isotope.

### Co-Immunoprecipitation.

500 g protein lysate extracted using lysis buffer were incubated with 5 μL pan-TEAD antibody (CST #13295, 33 g/ml) at 4 ° C overnight with rotation, and then incubated with 15 μL Protein G magnetic beads (Fisher #88847) at R.T. for 1 hour with rotation. After binding, beads were washed three times with lysis buffer and one time with ddH2O. IP samples were eluted using 1× gel loading buffer supplied with 20 mM DTT and boiled at 96 ° C for 10 min. 50 g protein lysate were used for input, and 3% IP flow-thru were used for binding efficiency control. *0 indicated DMSO control. For the detection process, primary antibodies used were: YAP/TAZ rabbit monoclonal antibody (mAb) at a dilution of 1:2000, CST #8418S; Pan-TEAD rabbit mAb at 1:2000, CST #13295S; and 3-actin mouse mAb at 1:8000, Sigma #A5316. Secondary antibodies applied in 1× TBST were: Rabbit IgG-HRP at 1:10000 and Mouse IgG-HRP also at 1:10000.

### Cell Lines.

HEK 293T (CRL-3216), Huh7 (CVCL_0336), HepG2 (HB-8065), and AML12 (CRL-2254) cell lines were bought from the American Type Culture Collection (ATCC). All cells were grown at 37 ° C with 5% C02 in media as recommended by the supplier.

### Luciferase Reporter Assay.

HEK 293T cells were plated at 2×10⁴ cells/well in a 96 well microplate and were transfected with 50 ng of 8×GTIIC-luciferase plasmid (Addgene #34615) and 0.5 ng of pRL-CMV (Promega #E226A) by using lipofectamine 3000 (Life Technologies #L3000). The 8×GTIIc-luciferase reporter is a Yap/Taz-activity reporter containing eight TEAD consensus binding sequences. After 6 h, transfected cells were treated with various concentrations of compounds or DMSO control. Luciferase signals were measured using the Dual Luciferase Reporter Assay System (Promega, E1960) according to the manufacturer's instructions. Experiments were performed in triplicate and repeated at least three times.

### RNA Extraction and RT-qPCR.

Huh7 and HepG2 liver cancer cells were seeded at 1×10⁶ cells/dish and allowed to adhere overnight. The cells were then treated with indicated doses of compounds in medium for 24 hours. The cells were then harvested, and total RNA was purified using the Aurum total RNA mini kit (Bio-Rad #732-6820) according to the manufacturer's instructions. Complementary DNA was synthesized from 500 ng total RNA template by iScript reverse transcription kit (Bio-Rad #1725037) according to the manufacturer's instructions. Real-time PCR reactions utilized 60 ng cDNA and PrimePCR primers from Bio-Rad (Unique Assay ID: human GAPDH-qHsaCEP0041396, human CTGF-qHsaCEP0024255, human CYR61-qHsaCEP0024230) designed for SYBR Green gene expression analysis (Bio-Rad #1726270). All reactions were carried out in triplicate using Quant Studio 3 PCR system (Applied Biosystems). Relative gene expression level was quantified via ΔΔCt method by using GAPDH as an endogenous reference.

### Western Blot Analysis.

Treated Huh7 cells were harvested and lysed in cold RIPA lysis buffer containing proteasome and phosphatase inhibitors. The protein concentrations were determined using the BCA Protein Assay kit. The prepared protein samples were loaded on and separated by 10% SDS-PAGE gel and then were transferred to PVDF membrane. Blots were blocked for 1 h at RT in blocking buffer and incubated with corresponding primary antibodies: anti-CTGF (ab227180), and anti-GAPDH (ab9485), at 4 ° C overnight. Then the blots were incubated with HRP-conjugated anti-rabbit secondary antibody in blocking buffer for 1.5 h at RT. The membranes were imaged with ChemiDoc MP imaging system.

### In Vitro Antiproliferative Assay.

CCK8 assay kit was used to determine antiproliferative activity of synthesized compounds against Huh7 cell line. First, Huh7 cells were seeded into 96-well plates at a density of 3000 cells/well and incubated overnight. Cells were then co-incubated with compounds at 8 concentrations for 72 h. Then, the cell proliferation was determined by the CCK8 kit according to the standard protocol. Experiments were performed in triplicate.

### Colony Formation Assay.

Huh7 or HepG2 (500 cells/well) were plated into 6 well plates (500 cells per well) and treated with 0 or 5 μM compound 22. Colonies were grown in the absence of soft agar for 2 weeks in a humidified atmosphere of 5% C02 at 37 ° C Crystal violet blue solution (Sigma) was used to stain the colonies for one hour and fixed in methanol followed by counting under a microscope. Triplicate wells were used for each cell line and three independent experiments were performed.

### Immunohistochemical Assay.

Tissues harvested from mice were fixed in 4% paraformaldehyde PBS solution for 24 hours, embedded in paraffin, and sectioned (6 μm). Standard staining protocols were performed for IHC using an ABC kit from Vector lab (Vector Laboratories, Inc., CA). The primary antibody was mouse anti-Ki67 antibody (catalogNCL-L-Ki67-MM1, 1:100 dilution) from Leica Biosystems (Deer Park, IL). Stained sections were detected using a microscope (Leica, Germany).

### Flow Cytometry Analysis of Cell Apoptosis and Cell Cycle.

Apoptosis was detected using an Annexin V-FITC apoptosis detection kit (Thermo Fisher Scientific) following the manufacturer’ instructions. Briefly, about 50000 cells were suspended in 500 μl of binding buffer. The cells were incubated with 5 μl FITC-conjugated Annexin V for 15 minutes and incubated with 5 μl propidium Iodide staining solution for 15 minutes in darkness at room temperature. Cell apoptosis was then analyzed by flow cytometry using a FACSCalibur flow cytometer (Becton Dickinson, San Jose, CA).

For cell cycle analysis, cells were fixed with pre-cold 70% ethanol for 30 minutes. Then the cells were washed with PBS and stained with propidium iodide working solution for 30 minutes in the dark. The stained cells were analyzed by flow cytometry. Cell cycle distribution was analyzed using FlowJo_V10 software. For each experiment, 20000 events per sample were recorded. Percentages of cells in G0/G1, S and G2/M phases were calculated and shown in DNA histograms.

### Animal Experimentation.

All animal protocols were approved by the Animal Care and Usage Committee at Tulane University and were conducted in compliance with their guidelines. In vivo xenograft study, immunodeficient NSG male mice (n=10, 6-week old) were purchased from Charles River Laboratories (Wilmington, MA) and were raised in an environment free of specific pathogens.

In vivo Xenograft Study.

Tumors were implanted by subcutaneous injection of 5×10\hat{ }6 Huh7 cells in 100 μL of PBS containing 10% matrix gel (Coming, NY, USA), into the right flank of the mouse. When the tumor volume reached about 85 mm³, mice were randomized into the vehicle control group (n=5) and a treatment group (n=5). The vehicle group (n=5) received the vehicle only (50% phosal 50 PG, 45% miglyol 810 N and 5% polysorbate 80), while the treatment group (n=5) was given compound 22 (50 mg/kg in 50% phosal 50 PG, 45% miglyol 810 N and 5% polysorbate 80) via IP injection every three days. After 24 days, mice were euthanized. Sorafenib (100 mg/kg, every three day) was dissolved in 75% Ethanol and Cremophor EL (1:1) and administered directly into the animals' stomachs using a gastric probe. Tumor size and body weight were measured every 3 days, and tumor volume was estimated using the formula volume=½×length×width². Tumor growth inhibition value (TGI) was calculated as TGI (%) [1−Vt/Vc]×100, where Vc and Vt were tumor volumes before and after administration.

### Drug Assessment in Murine HB Model.

For drug assessment in the murine HB model, plasmids carrying T3EF1α-YAPS127A (20 μg), pCMV-sleeping beauty transposase (SB, 0.8 μg), and pT3-EF1α-ΔN90-β-catenin (20 μg) from Addgene (Watertown, MA) were used. The plasmids were extracted using the Endotoxin-Free Maxiprep kit (Qiagen, Redwood City, CA), diluted in 2 ml 0.9% sodium chloride solution, and injected via hydrodynamic tail vein injection (HDTV) into C57BL6 mice (n=15, 6 week old). Three weeks post the HDTV injection, compound 22 (25 mg/g body weight) or mock control were applied twice a week for total 9 weeks via IP injection. Two days post last drug treatment, mice were euthanized and tumor tissues were subjected to molecular and histological characterizations.

### Statistical Analysis.

Statistical analyses were conducted in Prism 9 software. Results are reported as mean±SD. Student's t-test was utilized to compare individual data with control values for the analysis of statistical significance. *, P<0.05; **, P<0.01; ***, P<0.001; ****, P<0.0001; ns, P>0.05.

### REFERENCES

1. Yu, F. X.; Guan, K. L., The Hippo pathway: regulators and regulations. Genes Dev 2013, 27 (4), 355-71.
2. Yu, F. X.; Zhao, B.; Guan, K. L., Hippo Pathway in Organ Size Control, Tissue Homeostasis, and Cancer. Cell 2015,163 (4), 811-28.
3. Ma, S. H.; Meng, Z. P.; Chen, R.; Guan, K. L., The Hippo Pathway: Biology and Pathophysiology. Annu Rev Biochem 2019, 88, 577-604.
4. Dey, A.; Varelas, X.; Guan, K. L., Targeting the Hippo pathway in cancer, fibrosis, wound healing and regenerative medicine. Nat Rev Drug Discov 2020, 19 (7), 480-494.
5. Meng, Z. P.; Moroishi, T.; Guan, K. L., Mechanisms of Hippo pathway regulation. Gene Dev 2016, 30 (1), 1-17.
6. Avruch, J.; Zhou, D. W.; Fitamant, J.; Bardeesy, N.; Mou, F.; Barrufet, L. R., Protein kinases of the Hippo pathway: Regulation and substrates. Semin Cell Dev Biol 2012, 23 (7), 770-784.
7. Bae, S. J.; Luo, X. L., Activation mechanisms of the Hippo kinase signaling cascade. Bioscience Rep 2018, 38.
8. Lin, K. C.; Park, H. W.; Guan, K. L., Regulation of the Hippo Pathway Transcription Factor TEAD. Trends Biochem Sci 2017, 42 (11), 862-872.
9. Lamar, J. M.; Stern, P.; Liu, H.; Schindler, J. W.; Jiang, Z. G.; Hynes, R. O., The Hippo pathway target, YAP, promotes metastasis through its TEAD-interaction domain. P Natl Acad Sci USA 2012, 109 (37), E2441-E2450.
10. Lai, D.; Ho, K. C.; Hao, Y. W.; Yang, X. L., Taxol Resistance in Breast Cancer Cells Is Mediated by the Hippo Pathway Component TAZ and Its Downstream Transcriptional Targets Cyr61 and CTGF. Cancer Res 2011, 71 (7), 2728-2738.
11. Li, H. L.; Li, Q. Y.; Jin, M. J.; Lu, C. F.; Mu, Z. Y.; Xu, W. Y.; Song, J.; Zhang, Y.; Zhang, S. Y., A review: hippo signaling pathway promotes tumor invasion and metastasis by regulating target gene expression. J Cancer Res Clin 2021,147 (6), 1569-1585.
12. Lau, L. F., CCN1/CYR61: the very model of a modern matricellular protein. Cell Mol Life Sci 2011, 68 (19), 3149-3163.
13. Ramazani, Y.; Knops, N.; Elmonem, M. A.; Nguyen, T. O.; Arcolino, F. O.; van den Heuvel, L.; Levtchenko, E.; Kuypers, D.; Goldschmeding, R., Connective tissue growth factor (CTGF) from basics to clinics. Matrix Biol 2018, 68-69, 44-66.
14. Harvey, K. F.; Zhang, X. M.; Thomas, D. M., The Hippo pathway and human cancer. Nat Rev Cancer 2013, 13 (4), 246-257.
15. Pan, D. J., The Hippo Signaling Pathway in Development and Cancer. Dev Cell 2010, 19 (4), 491-505.
16. Johnson, R.; Halder, G., The two faces of Hippo: targeting the Hippo pathway for regenerative medicine and cancer treatment. Nat Rev Drug Discov 2014, 13 (1), 63-79.
17. Calses, P. C.; Crawford, J. J.; Lill, J. R.; Dey, A., Hippo Pathway in Cancer: Aberrant Regulation and Therapeutic Opportunities. Trends Cancer 2019, 5 (5), 297-307.
18. Moroishi, T.; Hayashi, T.; Pan, W. W.; Fujita, Y.; Holt, M. V.; Qin, J.; Carson, D. A.; Guan, K. L., The Hippo Pathway Kinases LATS½ Suppress Cancer Immunity. Cell 2016, 167 (6), 1525-+.
19. Pobbati, A. V.; Hong, W. J., A combat with the YAP/TAZ-TEAD oncoproteins for cancer therapy. Theranostics 2020, 10 (8), 3622-3635.
20. Ozakyol, A., Global Epidemiology of Hepatocellular Carcinoma (HCC Epidemiology). J Gastrointest Cancer 2017, 48 (3), 238-240.
21. Ahmad, M. I.; Khan, M. U.; Kodali, S.; Shetty, A.; Bell, S. M.; Victor, D., Hepatocellular Carcinoma Due to Nonalcoholic Fatty Liver Disease: Current Concepts and Future Challenges. J Hepatocell Carcino 2022, 9, 477-496.
22. Anstee, Q. M.; Reeves, H. L.; Kotsiliti, E.; Govaere, O.; Heikenwalder, M., From NASH to HCC: current concepts and future challenges. Nat Rev Gastro Hepat 2019, 16 (7), 411-428.
23. Colagrande, S.; Inghilesi, A. L.; Aburas, S.; Taliani, G. G.; Nardi, C.; Marra, F., Challenges of advanced hepatocellular carcinoma. World J Gastroenterol 2016, 22 (34), 7645-59.
24. Desai, J. R.; Ochoa, S.; Prins, P. A.; He, A. R., Systemic therapy for advanced hepatocellular carcinoma: an update. J Gastrointest Oncol 2017, 8 (2), 243-255.
25. Bruix, J.; Qin, S.; Merle, P.; Granito, A.; Huang, Y.-H.; Bodoky, G.; Pracht, M.; Yokosuka, O.; Rosmorduc, O.; Breder, V.; Gerolami, R.; Masi, G.; Ross, P. J.; Song, T.; Bronowicki, J.-P.; Ollivier-Hourmand, I.; Kudo, M.; Cheng, A.-L.; Llovet, J. M.; Finn, R. S.; LeBerre, M.-A.; Baumhauer, A.; Meinhardt, G.; Han, G., Regorafenib for patients with hepatocellular carcinoma who progressed on sorafenib treatment (RESORCE): a randomised, double-blind, placebo-controlled, phase 3 trial. The Lancet 2017, 389 (10064), 56-66.
26. Foerster, F.; Gairing, S. J.; Muller, L.; Galle, P. R., NAFLD-driven HCC: Safety and efficacy of current and emerging treatment options. J Hepatol 2022, 76 (2), 446-457.
27. Fornari, F.; Giovannini, C.; Piscaglia, F.; Gramantieri, L., Elucidating the Molecular Basis of Sorafenib Resistance in HCC: Current Findings and Future Directions. J Hepatocell Carcinoma 2021, 8, 741-757.
28. Reveron-Thornton, R. F.; Teng, M. L. P.; Lee, E. Y.; Tran, A.; Vajanaphanich, S.; Tan, E. X.; Nerurkar, S. N.; Ng, R. X.; Teh, R.; Tripathy, D. P.; Ito, T.; Tanaka, T.; Miyake, N.; Zou, B. Y.; Wong, C.; Toyoda, H.; Esquivel, C. O.; Bonham, C. A.; Nguyen, M. H.; Huang, D. Q., Global and regional long-term survival following resection for HCC in the recent decade: A meta-analysis of 110 studies. Hepatol Commun 2022, 6 (7), 1813-1826.
29. Sun, T.; Mao, W. H.; Peng, H.; Wang, Q.; Jiao, L., YAP promotes sorafenib resistance in hepatocellular carcinoma by upregulating survivin. Cell Oncol 2021, 44 (3), 689-699.
30. Joo, J. S.; Cho, S. Y.; Ron, W. S.; Kim, J. S.; Kang, S. H.; Lee, E. S.; Moon, H. S.; Kim, S. H.; Sung, J. K.; Kwon, I. S.; Eun, H. S.; Lee, B. S., TEAD2 as a novel prognostic factor for hepatocellular carcinoma. Oncol Rep 2020, 43 (6), 1785-1796.
31. Reggiani, F.; Gobbi, G.; Ciarrocchi, A.; Arnbrosetti, D. C.; Sancisi, V., Multiple roles and context-specific mechanisms underlying YAP and TAZ-mediated resistance to anti-cancer therapy. Bba-Rev Cancer 2020, 1873 (1).
32. Reggiani, F.; Gobbi, G.; Ciarrocchi, A.; Sancisi, V., YAP and TAZ Are Not Identical Twins. Trends Biochem Sci 2021, 46 (2), 154-168.
33. Holden, J. K.; Cunningham, C. N., Targeting the Hippo Pathway and Cancer through the TEAD Family of Transcription Factors. Cancers 2018, 10 (3).
34. Tian, W.; Yu, J. Z.; Tomchick, D. R.; Pan, D. J.; Luo, X. L., Structural and functional analysis of the YAP-binding domain of human TEAD2. P Natl Acad Sci USA 2010,107 (16), 7293-7298.
35. Chen, L. M.; Chan, S. W.; Zhang, X. Q.; Walsh, M.; Lim, C. J.; Hong, W. J.; Song, H. W., Structural basis of YAP recognition by TEAD4 in the Hippo pathway. Gene Dev 2010, 24 (3), 290-300.
36. Zagiel, B.; Melnyk, P.; Cotelle, P., Progress with YAP/TAZ-TEAD inhibitors: a patent review (2018-present). Expert Opin Ther Pat 2022, 32 (8), 899-912.
37. Liu-Chittenden, Y.; Huang, B.; Shim, J. S.; Chen, Q.; Lee, S. J.; Anders, R. A.; Liu, J. O.; Pan, D. J., Genetic and pharmacological disruption of the TEAD-YAP complex suppresses the oncogenic activity of YAP. Gene Dev 2012, 26 (12), 1300-1305.
38. Wang, L.; Kim, D.; Wise, J. T. F.; Shi, X. L.; Zhang, Z.; DiPaola, R. S., p62 as a therapeutic target for inhibition of autophagy in prostate cancer. Prostate 2018, 78 (5), 390-400.
39. Saunders, J. T.; Holmes, B.; Benavides-Serrato, A.; Kumar, S.; Nishimura, R. N.; Gera, J., Targeting the YAP-TEAD interaction interface for therapeutic intervention in glioblastoma. J Neuro-Oncol 2021, 152 (2), 217-231.
40. Crawford, J. J.; Bronner, S. M.; Zbieg, J. R., Hippo pathway inhibition by blocking the YAP/TAZ-TEAD interface: a patent review. Expert Opin Ther Pat 2018, 28 (12), 867-873.
41. Li, Z.; Zhao, B.; Wang, P.; Chen, F.; Dong, Z. H.; Yang, H. R.; Guan, K. L.; Xu, Y. H., Structural insights into the YAP and TEAD complex. Gene Dev 2010, 24 (3), 235-240.
42. Chen, L. M.; Loh, P. G.; Song, H. W., Structural and functional insights into the TEAD-YAP complex in the Hippo signaling pathway. Protein Cell 2010, 1 (12), 1073-1083.
43. Zhang, Z. Z.; Lin, Z. H.; Zhou, Z.; Shen, H. C.; Yan, S. F.; Mayweg, A. V.; Xu, Z. H.; Qin, N.; Wong, J. C.; Zhang, Z. S.; Rong, Y. P.; Fry, D. C.; Hu, T. S., Structure-Based Design and Synthesis of Potent Cyclic Peptides Inhibiting the YAP-TEAD Protein-Protein Interaction. Acs Medicinal Chemistry Letters 2014, 5 (9), 993-998.
44. Craik, D. J.; Fairlie, D. P.; Liras, S.; Price, D., The Future of Peptide-based Drugs. Chem Biol Drug Des 2013, 81 (1), 136-147.
45. Smith, S. A.; Sessions, R. B.; Shoemark, D. K.; Williams, C.; Ebrahimighaei, R.; McNeill, M. C.; Crump, M. P.; McKay, T. R.; Harris, G.; Newby, A. C.; Bond, M., Antiproliferative and Antimigratory Effects of a Novel YAP-TEAD Interaction Inhibitor Identified Using in Silico Molecular Docking. J Med Chem 2019, 62 (3), 1291-1305.
46. Sturbaut, M.; Bailly, F.; Coevoet, M.; Sileo, P.; Pugniere, M.; Liberelle, M.; Magnez, R.; Thuru, X.; Chartier-Harlin, M. C.; Melnyk, P.; Gelin, M.; Allemand, F.; Guichou, J. F.; Cotelle, P., Discovery of a cryptic site at the interface 2 of TEAD - Towards a new family of YAP/TAZ-TEAD inhibitors. Eur J Med Chem 2021, 226.
47. Kim, J.; Lim, H.; Moon, S.; Cho, S. Y.; Kim, M.; Park, J. H.; Park, H. W.; No, K. T., Hot Spot Analysis of YAP-TEAD Protein-Protein Interaction Using the Fragment Molecular Orbital Method and Its Application for Inhibitor Discovery. Cancers 2021, 13 (16).
48. Furet, P.; Bordas, V.; Le Douget, M.; Salem, B.; Mesrouze, Y.; Imbach-Weese, P.; Sellner, H.; Voegtle, M.; Soldermann, N.; Chapeau, E.; Wartmann, M.; Scheufler, C.; Fernandez, C.; Kallen, J.; Guagnano, V.; Chene, P.; Schmelzle, T., The First Class of Small Molecules Potently Disrupting the YAP-TEAD Interaction by Direct Competition. Chemmedchem 2022, 17 (19).
49. Noland, C. L.; Gierke, S.; Schnier, P. D.; Murray, J.; Sandoval, W. N.; Sagolla, M.; Dey, A.; Hannoush, R. N.; Fairbrother, W. J.; Cunningham, C. N., Palmitoylation of TEAD Transcription Factors Is Required for Their Stability and Function in Hippo Pathway Signaling. Structure 2016, 24 (1), 179-186.
50. Chan, P.; Han, X.; Zheng, B. H.; Deran, M.; Yu, J. Z.; Jarugumilli, G. K.; Deng, H.; Pan, D. J.; Luo, X. L.; Wu, X., Autopalmitoylation of TEAD proteins regulates transcriptional output of the Hippo pathway. Nature Chemical Biology 2016, 12 (4), 282-+.
51. Bum-Erdene, K.; Yeh, I. J.; Gonzalez-Gutierrez, G.; Ghozayel, M. K.; Pollok, K.; Meroueh, S. O., Small-Molecule Cyanamide Pan-TEADmiddotYAPI Covalent Antagonists. J Med Chem 2022.
52. Lu, W. C.; Wang, J.; Li, Y.; Tao, H. R.; Xiong, H.; Lian, F. L.; Gao, J.; Ma, H. N.; Lu, T.; Zhang, D.; Ye, X. Q.; Ding, H.; Yue, L. Y.; Zhang, Y. Y.; Tang, H. Y.; Zhang, N. X.; Yang, Y. X.; Jiang, H. L.; Chen, K. X.; Zhou, B.; Luo, C., Discovery and biological evaluation of vinylsulfonamide derivatives as highly potent, covalent TEAD autopalmitoylation inhibitors. Eur J Med Chem 2019, 184.
53. Sun, Y.; Hu, L.; Tao, Z. P.; Jarugumilli, G. K.; Erb, H.; Singh, A.; Li, Q.; Cotton, J. L.; Greninger, P.; Egan, R. K.; Ip, Y. T.; Benes, C. H.; Che, J. W.; Mao, J. H.; Wu, X., Pharmacological blockade of TEAD-YAP reveals its therapeutic limitation in cancer cells. Nat Commun 2022, 13 (1).
54. Tang, T. T.; Konradi, A. W.; Feng, Y.; Peng, X.; Mae, M. Y.; Lie, J.; Yu, F. X.; Guan, K. L.; Post, L., Small Molecule Inhibitors of TEAD Auto-palmitoylation Selectively Inhibit Proliferation and Tumor Growth of NF2-deficient Mesothelioma. Mol Cancer Ther 2021, 20 (6), 986-998.
55. Heinrich, T.; Peterson, C.; Schneider, R.; Garg, S.; Schwarz, D.; Gunera, J.; Seshire, A.; Kotzner, L.; Schlesiger, S.; Musil, D.; Schilke, H.; Doerfel, B.; Diehl, P.; Bopple, P.; Lemos, A. R.; Sousa, P. M. F.; Freire, F.; Bandeiras, T. M.; Carswell, E.; Pearson, N.; Sirohi, S.; Hooker, M.; Trivier, E.; Broome, R.; Balsiger, A.; Crowden, A.; Dillon, C.; Wienke, D., Optimization of TEAD P-Site Binding Fragment Hit into In vivo Active Lead MSC-4106. J Med Chem 2022.
56. Rezaei, M. A.; Li, Y. J.; Wu, D. P.; Li, X. L.; Li, C. L., Deep Learning in Drug Design: Protein-Ligand Binding Affinity Prediction. Ieee Acm T Comput Bi 2022, 19 (1), 407-417.
57. Pobbati, A. V.; Han, X.; Hung, A. W.; Weiguang, S.; Huda, N.; Chen, G. Y.; Kang, C. B.; Chia, C. S. B.; Luo, X. L.; Hong, W. J.; Poulsen, A., Targeting the Central Pocket in Human Transcription Factor TEAD as a Potential Cancer Therapeutic Strategy. Structure 2015, 23 (11), 2076-2086.
58. Sutanto, F.; Konstantinidou, M.; Domling, A., Covalent inhibitors: a rational approach to drug discovery. Rsc Med Chem 2020,11 (8), 876-884.
59. Bauer, R. A., Covalent inhibitors in drug discovery: from accidental discoveries to avoided liabilities and designed therapies. Drug Discov Today 2015, 20 (9), 1061-1073.
60. Singh, J., The Ascension of Targeted Covalent Inhibitors. J Med Chem 2022, 65 (8), 5886-5901.
61. Perra, A.; Kowalik, M. A.; Ghiso, E.; Ledda-Columbano, G. M.; Di Tommaso, L.; Angioni, M. M.; Raschioni, C.; Testore, E.; Roncalli, M.; Giordano, S.; Columbano, A., YAP activation is an early event and a potential therapeutic target in liver cancer development. J Hepatol 2014, 61 (5), 1088-1096.
62. Tschaharganeh, D. F.; Chen, X.; Latzko, P.; Malz, M.; Gaida, M. M.; Felix, K.; Ladu, S.; Singer, S.; Pinna, F.; Gretz, N.; Sticht, C.; Tomasi, M. L.; Delogu, S.; Evert, M.; Fan, B.; Ribback, S.; Jiang, L. J.; Brozzetti, S.; Bergmann, F.; Dombrowski, F.; Schirmacher, P.; Calvisi, D. F.; Breuhahn, K., Yes-Associated Protein Up-regulates Jagged-1 and Activates the NOTCH Pathway in Human Hepatocellular Carcinoma. Gastroenterology 2013, 144 (7), 1530-U368.
63. Makino, Y.; Hikita, H.; Kodama, T.; Shigekawa, M.; Yamada, R.; Sakamori, R.; Eguchi, H.; Morii, E.; Yokoi, H.; Mukoyama, M.; Hiroshi, S.; Tatsumi, T.; Takehara, T., CTGF Mediates Tumor-Stroma Interactions between Hepatoma Cells and Hepatic Stellate Cells to Accelerate HCC Progression. Cancer Res 2018, 78 (17), 4902-4914.
64. Abdeldayem, A.; Raouf, Y. S.; Constantinescu, S. N.; Moriggl, R.; Gunning, P. T., Advances in covalent kinase inhibitors. Chem Soc Rev 2020, 49 (9), 2617-2687.
65. Bradshaw, J. M.; McFarland, J. M.; Paavilainen, V. 0.; Bisconte, A.; Tam, D.; Phan, V. T.; Romanov, S.; Finkle, D.; Shu, J.; Patel, V.; Ton, T.; Li, X. Y.; Loughhead, D. G.; Nunn, P. A.; Karr, D. E.; Gerritsen, M. E.; Funk, J. 0.; Owens, T. D.; Verner, E.; Brameld, K. A.; Hill, R. J.; Goldstein, D. M.; Taunton, J., Prolonged and tunable residence time using reversible covalent kinase inhibitors. Nat Chem Biol 2015, 11 (7), 525-+.
66. Arnott, J. A.; Planey, S. L., The influence of lipophilicity in drug discovery and design. Expert Opin Drug Dis 2012, 7 (10), 863-875.
67. Bharti, S.; Bharti, J. N.; Sinha, A.; Yadav, T., Common and Rare Histological Variants of Hepatoblastoma in Children: A Pathological Diagnosis and Review of the Literature. Gastrointest Tumors 2021, 8 (2), 41-46.
68. Smith, J. L.; Rodriguez, T. C.; Mou, H. W.; Kwan, S. Y.; Pratt, H.; Zhang, X. 0.; Cao, Y. Y.; Liang, S. Q.; Ozata, D. M.; Yu, T. X.; Yin, Q. Z.; Hazeltine, M.; Weng, Z. P.; Sontheimer, E. J.; Xue, W., YAP1 Withdrawal in Hepatoblastoma Drives Therapeutic Differentiation of Tumor Cells to Functional Hepatocyte-Like Cells. Hepatology 2021, 73 (3), 1011-1027.
69. Timmins, P., Industry News update covering July 2021. Ther Deliv 2021,12 (11), 757-774.
70. Genentech Heterobifunctional Molecules as TEAD Inhibitors. WO2021178339, 2021.
71. Novartis Bifunctional Degraders Comprising a TEAD Binder. WO2023031801, 2023.
72. Schradinger, L. S. R.-S., LLC: New York, NY, USA, 2021.
73. Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K.; Shaw, D. E.; Francis, P.; Shenkin, P. S., Glide: A new approach for rapid, accurate docking and scoring. 1. Method and assessment of docking accuracy. J Med Chem 2004, 47 (7), 1739-1749.
74. Halgren, T. A.; Murphy, R. B.; Friesner, R. A.; Beard, H. S.; Frye, L. L.; Pollard, W. T.; Banks, J. L., Glide: A new approach for rapid, accurate docking and scoring. 2.

Enrichment factors in database screening. J Med Chem 2004, 47(7), 1750-1759.
75. Lipinski, C. A.; Lombardo, F.; Dominy, B. W.; Feeney, P. J., Experimental and computational approaches to estimate solubility and permeability in drug discovery and development settings. Adv Drug Deliver Rev 1997, 23 (1-3), 3-25.
76. Gleeson, M. P., Generation of a set of simple, interpretable ADMET rules of thumb. J Med Chem 2008, 51 (4), 817-834.
77. Franken, N. A. P.; Rodermond, H. M.; Stap, J.; Haveman, J.; van Bree, C., Clonogenic assay of cells in vitro. Nat Protoc 2006,1 (5), 2315-2319.
78. Tao, J. Y.; Calvisi, D. F.; Ranganathan, S.; Cigliano, A.; Zhou, L. L.; Singh, S.; Jiang, L. J.; Fan, B. A.; Terracciano, L.; Armeanu-Ebinger, S.; Ribback, S.; Dombrowski, F.; Evert, M.; Chen, X.; Monga, S. P. S., Activation of beta-Catenin and YapI in Human Hepatoblastoma and Induction of Hepatocarcinogenesis in Mice. Gastroenterology 2014, 147 (3), 690-701.
79. Zhou, W.; Li, Y. P.; Song, J. H.; Li, C. L., Fluorescence polarization assay for the identification and evaluation of inhibitors at YAP-TEAD protein-protein interface 3. Anal Biochem 2019, 586.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula II: 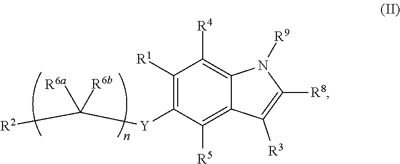
or a salt, solvate, hydrate, polymorph, co-crystal, tautomer, stereoisomer, isotopically labeled compound, or prodrug thereof,
wherein:
Y is —NR^{A}—, —O—, —S—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is 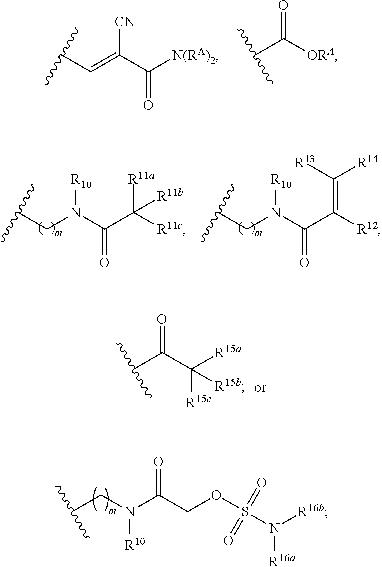
R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each of R^{16a} and R^{16b} is independently hydrogen, optionally substituted C₁₋₆ alkyl, or optionally substituted aryl;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

2. The compound of claim 1, or a salt, solvate, hydrate, polymorph, co-crystal, tautomer, stereoisomer, isotopically labeled compound, or prodrug thereof, wherein the compound is of Formula I: 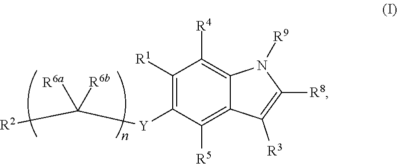
wherein:
Y is —NR^{A}—, —O—, —S—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is 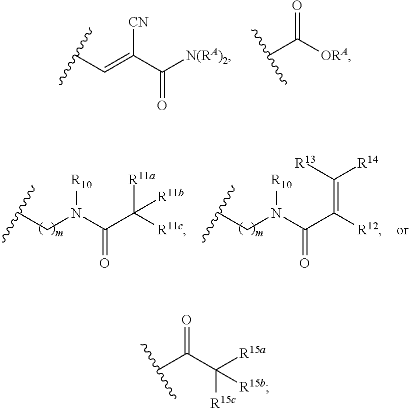
R⁴ is hydrogen or halogen;
R⁵ is hydrogen or halogen;
each of R^{6a} and R^{6b} is independently hydrogen, halogen, optionally substituted alkyl;
each of R^{7a} and R^{7b} is independently hydrogen, halogen, optionally substituted alkyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{1a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

3. The compound of claim 1, or a salt, solvate, hydrate, polymorph, co-crystal, tautomer, stereoisomer, isotopically labeled compound, or prodrug thereof, wherein the compound is of Formula I-A: 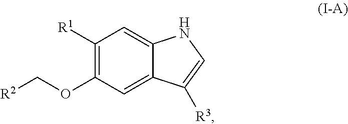
wherein:
R¹ is —Cl or —OMe;
R² is 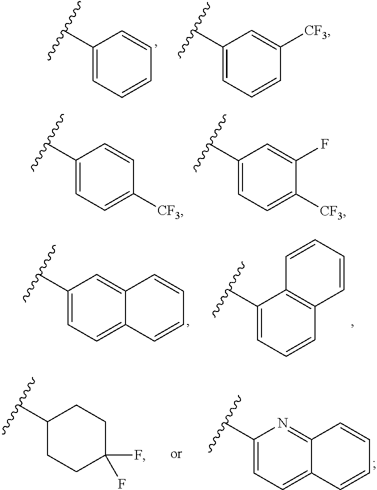
and
R³ is

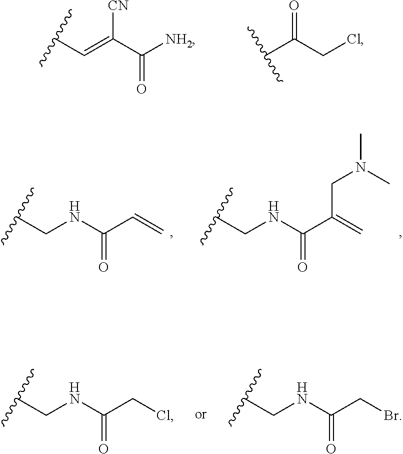

4. The compound of claim 1, or a salt, solvate, hydrate, polymorph, co-crystal, tautomer, stereoisomer, isotopically labeled compound, or prodrug thereof, wherein the compound is of Formula I-B: 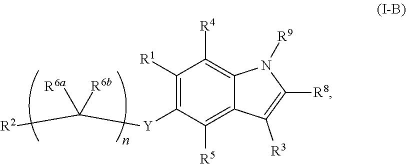
wherein:
Y is —NR^{A}—, —O—, or —CR^{7a}R^{7b}—;
R¹ is —F, —Br, —I, optionally substituted C₁₋₆ alkyl, optionally substituted carbocyclyl, —CN, —OR^{B}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is 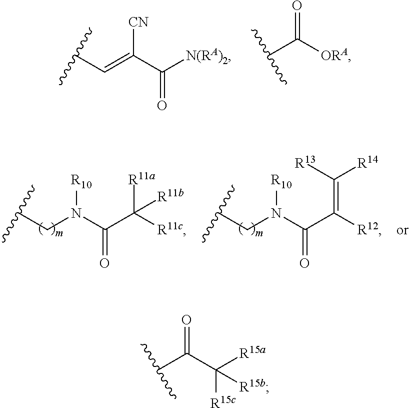
R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen or halogen;
each of R¹², R¹³, and R¹⁴ is independently hydrogen, halogen, optionally substituted C₁₋₆ alkyl, optionally substituted C₁₋₆ heteroalkyl, or —CN;
each of R^{15a}, R^{15b}, and R^{15c}, is independently hydrogen or halogen, provided that at least one of R^{15a}, R^{15b}, and R^{15c} is halogen;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{B} is independently hydrogen, optionally substituted C₂-C₁₀ alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
n is an integer from 0 to 1; and
m is an integer from 0 to 1.

5. The compound of claim 1, or a salt, solvate, hydrate, polymorph, co-crystal, tautomer, stereoisomer, isotopically labeled compound, or prodrug thereof, wherein the compound is of Formula I-C: 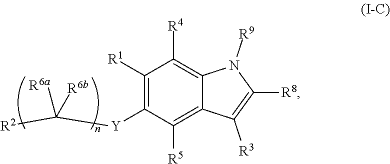
wherein:
Y is —NR^{A}—, —O—, or —CR^{7a}R^{7b}—;
R¹ is halogen, optionally substituted C₁₋₆ alkyl, optionally substituted carbocyclyl, —CN, —OR^{A}, —C(═O)OR^{A}, —Si(R^{A})₃, or —S(═O)₂R^{A};
R² is optionally substituted C₁₋₆ alkyl, —OR^{A}, optionally substituted carbocyclyl, optionally substituted heterocyclyl, optionally substituted aryl, or optionally substituted heteroaryl;
R³ is 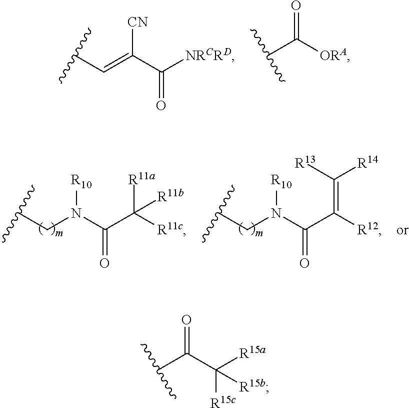
R⁴ is hydrogen or halogen;
R⁵ is hydrogen or halogen;
each of R^{6a} and R^{6b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
each of R^{7a} and R^{7b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl;
R⁸ is hydrogen, optionally substituted C₁₋₆ alkyl, —CN, —OR^{A}, —C(═O)OR^{A}, or —C(═O)N(R^{A})₂;
R⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group;
R¹⁰ is hydrogen or optionally substituted C₁₋₆ alkyl;
each of R^{11a}, R^{11b}, and R^{11c} is independently hydrogen, —F, or —I;
R¹² is independently hydrogen, halogen, or —CN;
each of R¹³ and R¹⁴ is independently hydrogen, halogen, optionally substituted C₁₋₆ alkyl, optionally substituted C₁₋₆ heteroalkyl, or —CN;
provided that at least one of R¹², R¹³, and R¹⁴ is halogen, optionally substituted C₁₋₆ alkyl, optionally substituted C₁₋₆ heteroalkyl, or —CN;
each of R^{15a}, R^{15b}, and R^{15c}, is independently hydrogen, —F, —Br, or —I, provided that at least one of R^{15a}, R^{15b}, and R^{15c} is —F, —Br, or —I;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring;
R^{C} is optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring;
R^{D} is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or R^{C} and R^{D} are joined together with the intervening atom to form an optionally substituted, monocyclic, heterocyclic or heteroaryl ring;
n is an integer from 0 to 1; and
m is an integer from 0 to 1.

6. The compound of any of claims 1-4, or a salt, solvate, hydrate, polymorph, co-crystal, tautomer, stereoisomer, isotopically labeled compound, or prodrug thereof, wherein Y is —NH—, —O—, —S—, or —CH₂—.

7. The compound of any of claims 1-5, or a salt, solvate, hydrate, polymorph, co-crystal, tautomer, stereoisomer, isotopically labeled compound, or prodrug thereof, wherein Y is —O—.

8. The compound of any of claims 1-4, or a salt, solvate, hydrate, polymorph, co-crystal, tautomer, stereoisomer, isotopically labeled compound, or prodrug thereof, wherein Y is —NH—, —NMe—, —S—, or —CH₂—.

9. The compound of any of claims 1-7, or a salt, solvate, hydrate, polymorph, co-crystal, tautomer, stereoisomer, isotopically labeled compound, or prodrug thereof, wherein R¹ is halogen, —CN, C₁₋₄ alkyl optionally substituted with one or more fluorine atoms, cyclopropyl, —C(═O)O(C₁₋₃ alkyl), —O(C₁₋₃ alkyl optionally substituted with one or more fluorine atoms), —Si(C₁₋₃ alkyl)₃, or —S(═O)₂(C₁₋₃ alkyl).

10. The compound of any of claims 1-9, or a salt, solvate, hydrate, polymorph, co-crystal, tautomer, stereoisomer, isotopically labeled compound, or prodrug thereof, wherein R¹ is halogen or —O(C₁₋₃ alkyl optionally substituted with one or more fluorine atoms).

11. The compound of any of claims 1-10, or a salt, solvate, hydrate, polymorph, co-crystal, tautomer, stereoisomer, isotopically labeled compound, or prodrug thereof, wherein R¹ is —Cl.

12. The compound of any of claims 1-10, or a salt, solvate, hydrate, polymorph, co-crystal, tautomer, stereoisomer, isotopically labeled compound, or prodrug thereof, wherein R¹ is halogen or —OMe.

13. The compound of any of claims 1-10, or a salt, solvate, hydrate, polymorph, co-crystal, tautomer, stereoisomer, isotopically labeled compound, or prodrug thereof, wherein R¹ is —F, —Br, or —I.

14. The compound of any of claims 1-10, or a salt, solvate, hydrate, polymorph, co-crystal, tautomer, stereoisomer, isotopically labeled compound, or prodrug thereof, wherein R¹ is —O(C₂₋₃ alkyl optionally substituted with one or more fluorine atoms).

15. The compound of any of claims 1-9, or a salt, solvate, hydrate, polymorph, co-crystal, tautomer, stereoisomer, isotopically labeled compound, or prodrug thereof, wherein R¹ is —F, —Br, —I, —CN, C₁₋₄ alkyl optionally substituted with one or more fluorine atoms, cyclopropyl, —C(═O)O(C₁₋₃ alkyl), —O(C₂₋₃ alkyl optionally substituted with one or more fluorine atoms), —Si(C₁₋₃ alkyl)₃, or —S(═O)₂(C₁₋₃ alkyl).

16. The compound of any of claims 1-9, or a salt, solvate, hydrate, polymorph, co-crystal, tautomer, stereoisomer, isotopically labeled compound, or prodrug thereof, wherein R¹ is —CN, C₁₋₄ alkyl optionally substituted with one or more fluorine atoms, cyclopropyl, —C(═O)O(C₁₋₃ alkyl), —Si(C₁₋₃ alkyl)₃, or —S(═O)₂(C₁₋₃ alkyl).

17. The compound of any of claims 1-16, or a salt, solvate, hydrate, polymorph, co-crystal, tautomer, stereoisomer, isotopically labeled compound, or prodrug thereof, wherein R² is C₁₋₆ alkyl optionally substituted with one or more fluorine atoms, —O(C₁₋₅ alkyl optionally substituted with one or more fluorine atoms), optionally substituted C₆₋₁₀ aryl, optionally substituted 5-10-membered heteroaryl, optionally substituted C₄₋₈ cycloalkyl, or optionally substituted 3-7-membered heterocyclyl.

18. The compound of any of claims 1-17, or a salt, solvate, hydrate, polymorph, co-crystal, tautomer, stereoisomer, isotopically labeled compound, or prodrug thereof, wherein R² is phenyl, wherein the phenyl is optionally substituted by one or more instances of halogen; C₁₋₄ alkyl optionally substituted with one or more fluorine atoms; C₃₋₆ cycloalkyl; —O(C₁₋₄ alkyl optionally substituted with one or more fluorine atoms); pentafluorosulfanyl; —CN; —Si(C₁₋₃ alkyl); —S(═O)₂(C₁₋₃ alkyl); phenyl optionally substituted by one or more —CF₃ groups; —CO₂(C₁₋₄ alkyl); or —S(C₁₋₄alkyl).

19. The compound of any of claims 1-17, or a salt, solvate, hydrate, polymorph, co-crystal, tautomer, stereoisomer, isotopically labeled compound, or prodrug thereof, wherein R² is C₄₋₈ cycloalkyl optionally substituted by halogen or C₁₋₄ alkyl optionally substituted with one or more fluorine atoms.

20. The compound of any of claims 1-17, or a salt, solvate, hydrate, polymorph, co-crystal, tautomer, stereoisomer, isotopically labeled compound, or prodrug thereof, wherein R² is

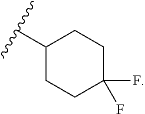 [SMILES: *C1CCC(F)(F)CC1]

21. The compound of any of claims 1-17, or a salt, solvate, hydrate, polymorph, co-crystal, tautomer, stereoisomer, isotopically labeled compound, or prodrug thereof, wherein R² is 3-7-membered heterocyclyl optionally substituted by halogen, C₁₋₄ fluoroalkyl, or phenyl optionally substituted by one or more fluorine atoms or —CF₃ groups.

22. The compound of any of claims 1-17, or a salt, solvate, hydrate, polymorph, co-crystal, tautomer, stereoisomer, isotopically labeled compound, or prodrug thereof, wherein R² is optionally substituted phenyl or optionally substituted naphthyl.

23. The compound of any of claims 1-18, or a salt, solvate, hydrate, polymorph, co-crystal, tautomer, stereoisomer, isotopically labeled compound, or prodrug thereof, wherein R² is phenyl optionally substituted either with halogen or with C₁₋₄ alkyl optionally substituted with one or more halogen atoms.

24. The compound of any of claims 1-18, or a salt, solvate, hydrate, polymorph, co-crystal, tautomer, stereoisomer, isotopically labeled compound, or prodrug thereof, wherein R² is phenyl optionally substituted with —F, —CF₃, —CHF₂, —CH₂F, or —Me.

25. The compound of any of claims 1-17, or a salt, solvate, hydrate, polymorph, co-crystal, tautomer, stereoisomer, isotopically labeled compound, or prodrug thereof, wherein R² is naphthyl optionally substituted either with halogen or with C₁₋₄ alkyl optionally substituted with one or more halogen atoms.

26. The compound of any of claims 1-17, or a salt, solvate, hydrate, polymorph, co-crystal, tautomer, stereoisomer, isotopically labeled compound, or prodrug thereof, wherein R² is naphthyl optionally substituted with —F, —CF₃, —CHF₂, —CH₂F, or —Me.

27. The compound of any of claims 1-17, or a salt, solvate, hydrate, polymorph, co-crystal, tautomer, stereoisomer, isotopically labeled compound, or prodrug thereof, wherein R² is

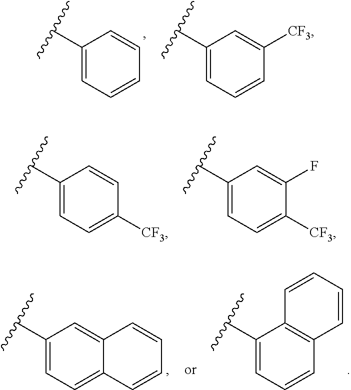

28. The compound of any of claims 1-18, or a salt, solvate, hydrate, polymorph, co-crystal, tautomer, stereoisomer, isotopically labeled compound, or prodrug thereof, wherein R² is CF₃ or CF₃

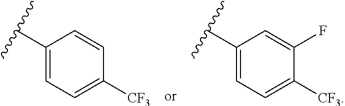 [SMILES: *c1ccc(C(F)(F)F)cc1 ; "or" ; *c1ccc(C(F)(F)F)c(F)c1]

29. The compound of any of claims 1-28, or a salt, solvate, hydrate, polymorph, co-crystal, tautomer, stereoisomer, isotopically labeled compound, or prodrug thereof, wherein R³ is

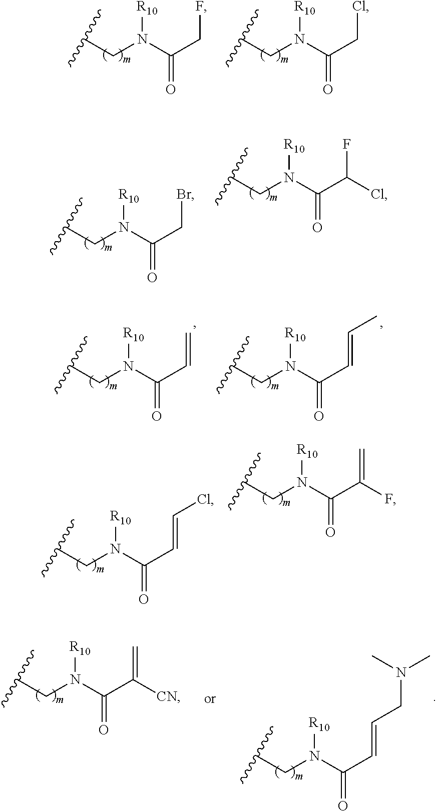

30. The compound of any of claims 1-29, or a salt, solvate, hydrate, polymorph, co-crystal, tautomer, stereoisomer, isotopically labeled compound, or prodrug thereof, wherein R³ is

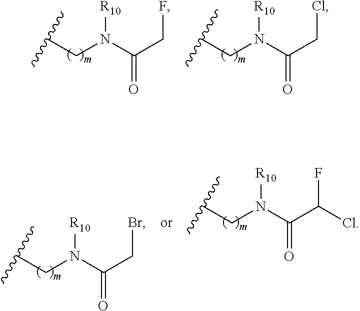

31. The compound of any of claims 1-28, or a salt, solvate, hydrate, polymorph, co-crystal, tautomer, stereoisomer, isotopically labeled compound, or prodrug thereof, wherein R³ is

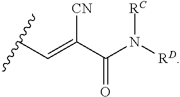

32. The compound of any of claims 1-28, or a salt, solvate, hydrate, polymorph, co-crystal, tautomer, stereoisomer, isotopically labeled compound, or prodrug thereof, wherein R³ is

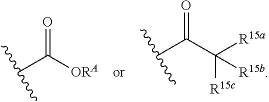

33. The compound of any of claims 1-28, or a salt, solvate, hydrate, polymorph, co-crystal, tautomer, stereoisomer, isotopically labeled compound, or prodrug thereof, wherein R³ is

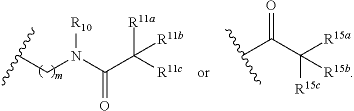

34. The compound of any one of claims 1-28, wherein R³ is

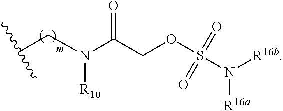

35. The compound of claim 34, wherein R³ is 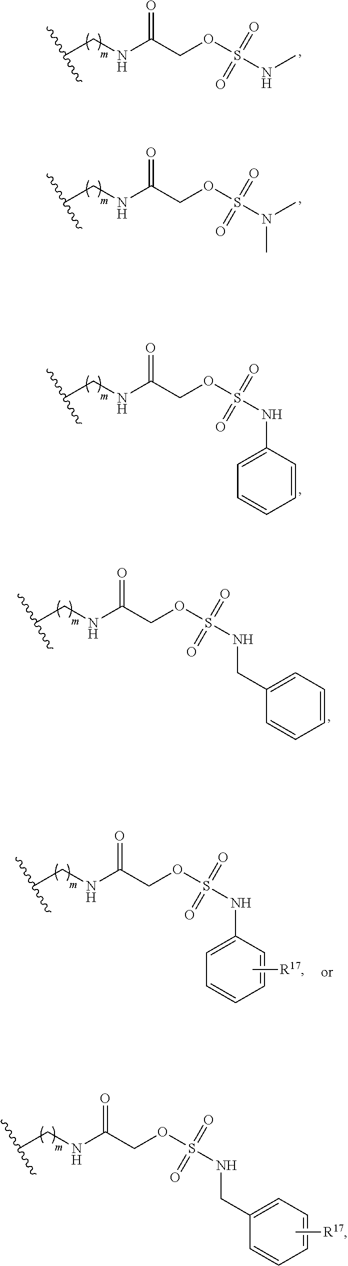
wherein R¹⁷ is halogen, —CN, optionally substituted C₁₋₂ alkyl, —OH, —O(optionally substituted C₁₋₂ alkyl), —C(═O)O(optionally substituted C₁₋₂ alkyl), or —C(═O)NH₂.

36. The compound of any of claims 1-28, or a salt, solvate, hydrate, polymorph, co-crystal, tautomer, stereoisomer, isotopically labeled compound, or prodrug thereof, wherein R³ is

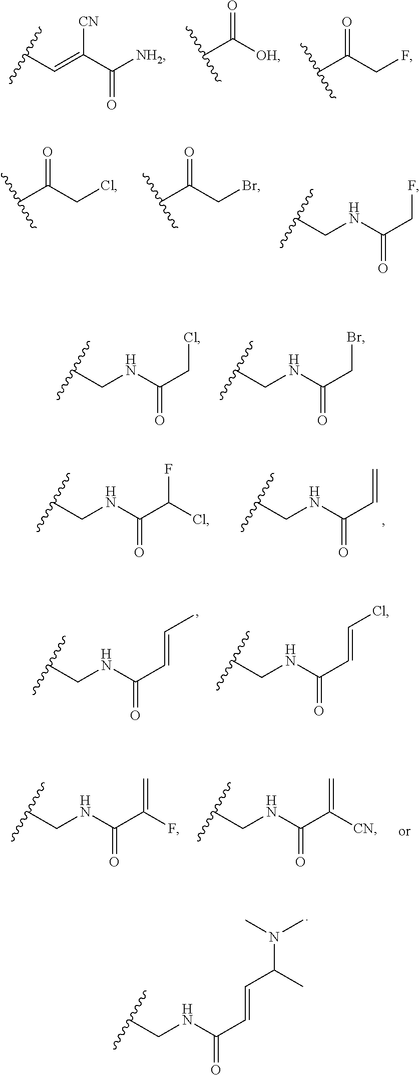

37. The compound of claim 36, or a salt, solvate, hydrate, polymorph, co-crystal, tautomer, stereoisomer, isotopically labeled compound, or prodrug thereof, wherein
R³ is

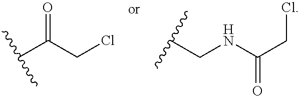 [SMILES: *C(=O)CCl ; *CNC(=O)CCl]

38. The compound of any of claims 1-37, or a salt, solvate, hydrate, polymorph, co-crystal, tautomer, stereoisomer, isotopically labeled compound, or prodrug thereof, wherein R⁴ is hydrogen.

39. The compound of any of claims 1-38, or a salt, solvate, hydrate, polymorph, co-crystal, tautomer, stereoisomer, isotopically labeled compound, or prodrug thereof, wherein R⁴ is fluorine.

40. The compound of any of claims 1-39, or a salt, solvate, hydrate, polymorph, co-crystal, tautomer, stereoisomer, isotopically labeled compound, or prodrug thereof, wherein R⁵ is hydrogen.

41. The compound of any of claims 1-39, or a salt, solvate, hydrate, polymorph, co-crystal, tautomer, stereoisomer, isotopically labeled compound, or prodrug thereof, wherein R⁵ is fluorine.

42. The compound of any of claims 1-41, or a salt, solvate, hydrate, polymorph, co-crystal, tautomer, stereoisomer, isotopically labeled compound, or prodrug thereof, wherein R^{6a} and R^{6b} are each independently hydrogen, halogen, or C₁₋₃ alkyl.

43. The compound of any of claims 1-42, or a salt, solvate, hydrate, polymorph, co-crystal, tautomer, stereoisomer, isotopically labeled compound, or prodrug thereof, wherein R^{6a} and R^{6b} are each hydrogen.

44. The compound of any of claims 1-43, or a salt, solvate, hydrate, polymorph, co-crystal, tautomer, stereoisomer, isotopically labeled compound, or prodrug thereof, wherein R^{7a} and R^{7b} are each independently hydrogen, halogen, or C₁₋₃ alkyl.

45. The compound of any of claims 1-44, or a salt, solvate, hydrate, polymorph, co-crystal, tautomer, stereoisomer, isotopically labeled compound, or prodrug thereof, wherein R^{7a} and R^{7b} are each hydrogen.

46. The compound of any of claims 1-45, or a salt, solvate, hydrate, polymorph, co-crystal, tautomer, stereoisomer, isotopically labeled compound, or prodrug thereof, wherein R⁸ is hydrogen, C₁₋₃ alkyl, —CN, —OH, —O(C₁₋₃ alkyl), —C(═O)O(C₁₋₂ alkyl), or —CONH₂.

47. The compound of any of claims 1-46, or a salt, solvate, hydrate, polymorph, co-crystal, tautomer, stereoisomer, isotopically labeled compound, or prodrug thereof, wherein R⁸ is hydrogen.

48. The compound of any of claims 1-47, or a salt, solvate, hydrate, polymorph, co-crystal, tautomer, stereoisomer, isotopically labeled compound, or prodrug thereof, wherein R⁹ is hydrogen, optionally substituted alkyl, or a nitrogen protecting group.

49. The compound of any of claims 1-48, or a salt, solvate, hydrate, polymorph, co-crystal, tautomer, stereoisomer, isotopically labeled compound, or prodrug thereof, wherein R⁹ is hydrogen.

50. The compound of any of claims 1-49, or a salt, solvate, hydrate, polymorph, co-crystal, tautomer, stereoisomer, isotopically labeled compound, or prodrug thereof, wherein R¹⁰ is —Me.

51. The compound of any of claims 1-49, or a salt, solvate, hydrate, polymorph, co-crystal, tautomer, stereoisomer, isotopically labeled compound, or prodrug thereof, wherein R¹⁰ is hydrogen.

52. The compound of any of claims 1-51, or a salt, solvate, hydrate, polymorph, co-crystal, tautomer, stereoisomer, isotopically labeled compound, or prodrug thereof, wherein at least one instance of R^{11a}, R^{11b}, and R^{11c} is halogen.

53. The compound of any of claims 1-52, or a salt, solvate, hydrate, polymorph, co-crystal, tautomer, stereoisomer, isotopically labeled compound, or prodrug thereof, wherein R¹² is hydrogen, halogen, or —CN.

54. The compound of any of claims 1-53, or a salt, solvate, hydrate, polymorph, co-crystal, tautomer, stereoisomer, isotopically labeled compound, or prodrug thereof, wherein R¹² is hydrogen.

55. The compound of any of claims 1-52, or a salt, solvate, hydrate, polymorph, co-crystal, tautomer, stereoisomer, isotopically labeled compound, or prodrug thereof, wherein R¹² is optionally substituted C₁₋₆ heteroalkyl.

56. The compound of claim 55, or a salt, solvate, hydrate, polymorph, co-crystal, tautomer, stereoisomer, isotopically labeled compound, or prodrug thereof, wherein R¹² is —CH₂NMe₂.

57. The compound of any of claims 1-56, or a salt, solvate, hydrate, polymorph, co-crystal, tautomer, stereoisomer, isotopically labeled compound, or prodrug thereof, wherein R¹³ is hydrogen.

58. The compound of any of claims 1-57, or a salt, solvate, hydrate, polymorph, co-crystal, tautomer, stereoisomer, isotopically labeled compound, or prodrug thereof, wherein R¹⁴ is hydrogen, halogen, optionally substituted C₁₋₆ alkyl, or optionally substituted C₁₋₆ heteroalkyl.

59. The compound of any of claims 1-58, or a salt, solvate, hydrate, polymorph, co-crystal, tautomer, stereoisomer, isotopically labeled compound, or prodrug thereof, wherein R¹⁴ is hydrogen.

60. The compound of any of claims 1-59, or a salt, solvate, hydrate, polymorph, co-crystal, tautomer, stereoisomer, isotopically labeled compound, or prodrug thereof, wherein R¹³ and R¹⁴ are hydrogen.

61. The compound of any of claims 1-60, or a salt, solvate, hydrate, polymorph, co-crystal, tautomer, stereoisomer, isotopically labeled compound, or prodrug thereof, wherein one of R^{15a}, R^{15b}, and R^{15c} is halogen.

62. The compound of any of claims 1-61, or a salt, solvate, hydrate, polymorph, co-crystal, tautomer, stereoisomer, isotopically labeled compound, or prodrug thereof, wherein n is 0.

63. The compound of any of claims 1-61, or a salt, solvate, hydrate, polymorph, co-crystal, tautomer, stereoisomer, isotopically labeled compound, or prodrug thereof, wherein n is 1.

64. The compound of any of claims 1-63, or a salt, solvate, hydrate, polymorph, co-crystal, tautomer, stereoisomer, isotopically labeled compound, or prodrug thereof, wherein m is 0.

65. The compound of any of claims 1-63, or a salt, solvate, hydrate, polymorph, co-crystal, tautomer, stereoisomer, isotopically labeled compound, or prodrug thereof, wherein m is 1.

66. The compound of any of claims 1-4, or a salt, solvate, hydrate, polymorph, co-crystal, tautomer, stereoisomer, isotopically labeled compound, or prodrug thereof, wherein the compound is any one of the formulae shown in Table 1:
TABLE 1

| | |
|---|---|
| 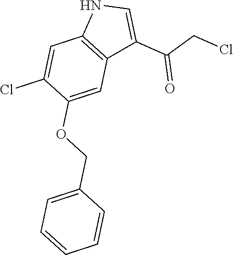 [SMILES: O=C(CCl)c1c[nH]c2cc(Cl)c(OCc3ccccc3)cc12] | 1 |
| 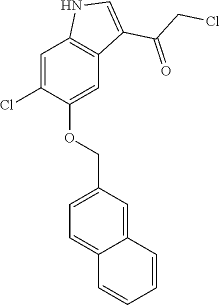 [SMILES: O=C(CCl)c1c[nH]c2cc(Cl)c(OCc3ccc4ccccc4c3)cc12] | 2 |
| 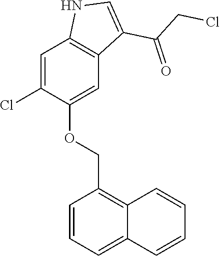 [SMILES: O=C(CCl)c1c[nH]c2cc(Cl)c(OCc3cccc4ccccc34)cc12] | 3 |
| 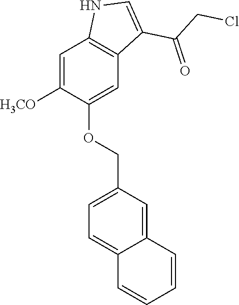 [SMILES: COc1cc2[nH]cc(C(=O)CCl)c2cc1OCc1ccc2ccccc2c1] | 5 |
| 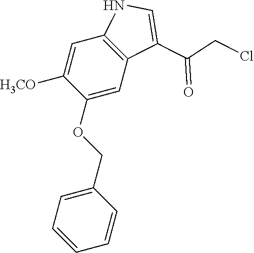 [SMILES: COc1cc2[nH]cc(C(=O)CCl)c2cc1OCc1ccccc1] | 4 |
| 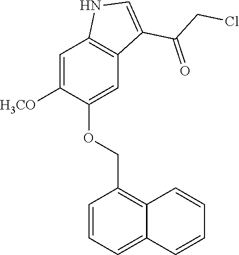 [SMILES: COc1cc2[nH]cc(C(=O)CCl)c2cc1OCc1cccc2ccccc12] | 6 |
| 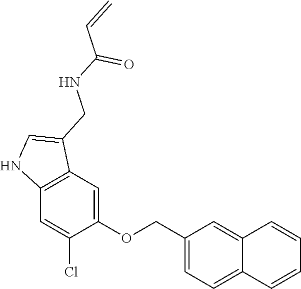 [SMILES: C=CC(=O)NCc1c[nH]c2cc(Cl)c(OCc3ccc4ccccc4c3)cc12] | 9 |
| 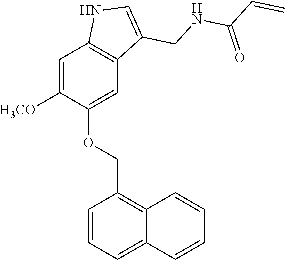 [SMILES: C=CC(=O)NCc1c[nH]c2cc(OC)c(OCc3cccc4ccccc34)cc12] | 10 |
| 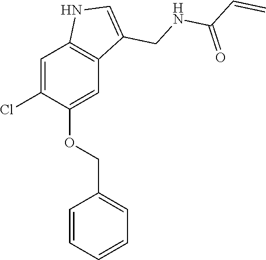 [SMILES: C=CC(=O)NCc1c[nH]c2cc(Cl)c(OCc3ccccc3)cc12] | 11 |
| 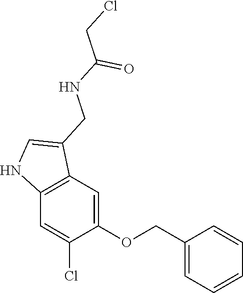 [SMILES: O=C(CCl)NCc1c[nH]c2cc(Cl)c(OCc3ccccc3)cc12] | 12 |
| 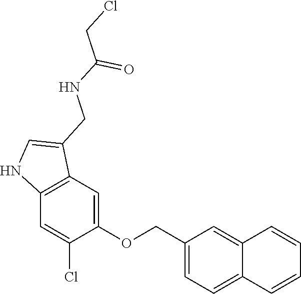 [SMILES: O=C(CCl)NCc1c[nH]c2cc(Cl)c(OCc3ccc4ccccc4c3)cc12] | 13 |
| 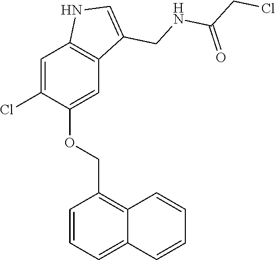 [SMILES: O=C(CCl)NCc1c[nH]c2cc(Cl)c(OCc3cccc4ccccc34)cc12] | 14 |
| 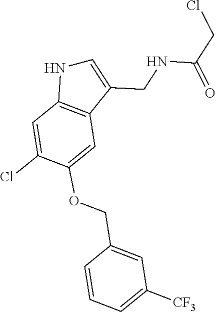 [SMILES: O=C(CCl)NCc1c[nH]c2cc(Cl)c(OCc3cccc(C(F)(F)F)c3)cc12] | 15 |
| 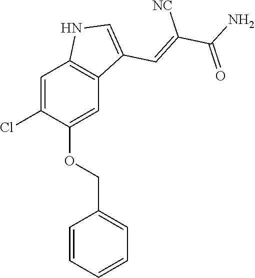 [SMILES: N#C/C(=C\c1c[nH]c2cc(Cl)c(OCc3ccccc3)cc12)C(N)=O] | 16 |
| 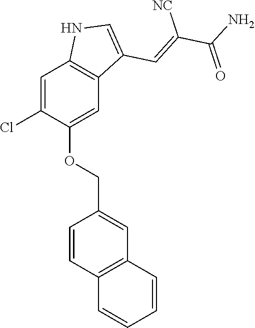 [SMILES: N#C/C(=C\c1c[nH]c2cc(Cl)c(OCc3ccc4ccccc4c3)cc12)C(N)=O] | 17 |
| 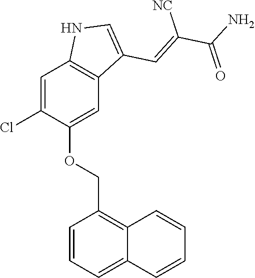 [SMILES: N#C/C(=C\c1c[nH]c2cc(Cl)c(OCc3cccc4ccccc34)cc12)C(N)=O] | 18 |
| 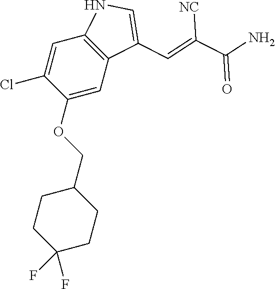 [SMILES: N#C/C(=C\c1c[nH]c2cc(Cl)c(OCC3CCC(F)(F)CC3)cc12)C(N)=O] | 19 |
| 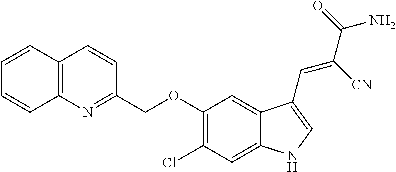 [SMILES: N#C/C(=C\c1c[nH]c2cc(Cl)c(OCc3ccc4ccccc4n3)cc12)C(N)=O] | 20 |
| 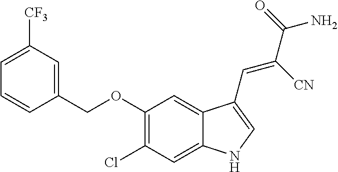 [SMILES: N#C/C(=C\c1c[nH]c2cc(Cl)c(OCc3cccc(C(F)(F)F)c3)cc12)C(N)=O] | 21 |
| 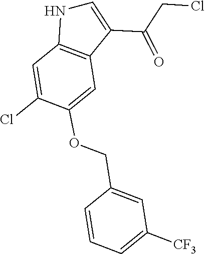 [SMILES: O=C(CCl)c1c[nH]c2cc(Cl)c(OCc3cccc(C(F)(F)F)c3)cc12] | 22 |
| 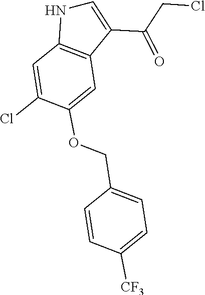 [SMILES: O=C(CCl)c1c[nH]c2cc(Cl)c(OCc3ccc(C(F)(F)F)cc3)cc12] | 23 |
| 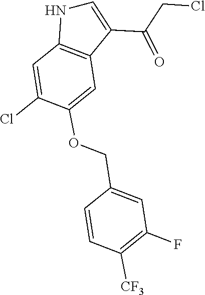 [SMILES: O=C(CCl)c1c[nH]c2cc(Cl)c(OCc3ccc(C(F)(F)F)c(F)c3)cc12] | 24 |
| 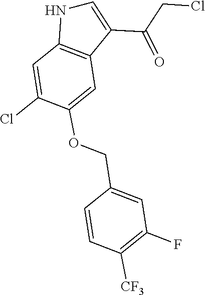 [SMILES: O=C(CCl)c1c[nH]c2cc(Cl)c(OCc3ccc(C(F)(F)F)c(F)c3)cc12] | CV-4-21 |
| 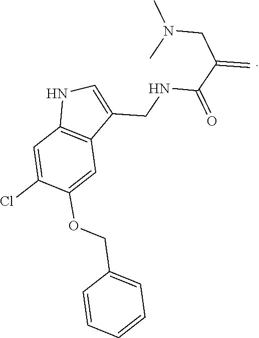 [SMILES: C=C(CN(C)C)C(=O)NCc1c[nH]c2cc(Cl)c(OCc3ccccc3)cc12] | 25 |

67. The compound of any one of claims 1-4, or a salt, solvate, hydrate, polymorph, co-crystal, tautomer, stereoisomer, isotopically labeled compound, or prodrug thereof, wherein the compound is not any one of the formulae shown in Table 1.

68. The compound of any one of claims 1-4, or a salt, solvate, hydrate, polymorph, co-crystal, tautomer, stereoisomer, isotopically labeled compound, or prodrug thereof, wherein the compound is not of Formula I-A.

69. A compound of formula 7: 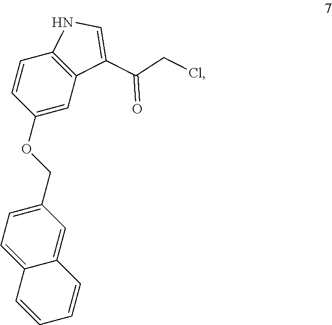 [SMILES: O=C(CCl)c1c[nH]c2ccc(OCc3ccc4ccccc4c3)cc12]
or a salt, solvate, hydrate, polymorph, co-crystal, tautomer, stereoisomer, isotopically labeled compound, or prodrug thereof.

70. A compound of formula 12: 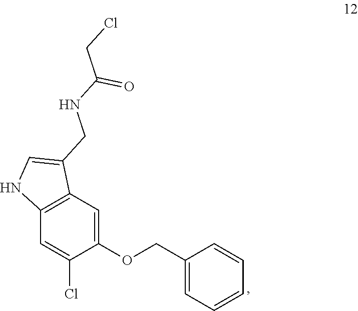 [SMILES: O=C(CCl)NCc1c[nH]c2cc(Cl)c(OCc3ccccc3)cc12]
or a salt, solvate, hydrate, polymorph, co-crystal, tautomer, stereoisomer, isotopically labeled compound, or prodrug thereof.

71. A compound of formula 22: 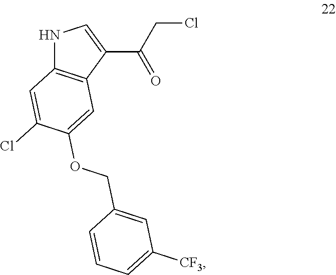 [SMILES: O=C(CCl)c1c[nH]c2cc(Cl)c(OCc3cccc(C(F)(F)F)c3)cc12]
or a salt, solvate, hydrate, polymorph, co-crystal, tautomer, stereoisomer, isotopically labeled compound, or prodrug thereof.

72. A pharmaceutical composition comprising:
the compound of any one of claims 1-71, or a salt, solvate, hydrate, polymorph, co-crystal, tautomer, stereoisomer, isotopically labeled compound, or prodrug thereof; and
optionally a pharmaceutically acceptable excipient.

73. The pharmaceutical composition of claim 72, further comprising an additional pharmaceutical agent.

74. The pharmaceutical composition of claim 73, wherein the additional pharmaceutical agent is Sorafenib.

75. The pharmaceutical composition of any one of claims 72-74, wherein the pharmaceutical composition is formulated for oral administration, intraperitoneal injection, or subcutaneous injection.

76. A kit comprising:
the compound of any one of claims 1-71, or a salt, solvate, hydrate, polymorph, co-crystal, tautomer, stereoisomer, isotopically labeled compound, or prodrug thereof, or the pharmaceutical composition of any one of claims 72-75; and
instructions for using the compound, or salt, solvate, hydrate, polymorph, co-crystal, tautomer, stereoisomer, isotopically labeled compound, or prodrug thereof, or pharmaceutical composition.

77. A method of treating a disease in a subject in need thereof, the method comprising administering to the subject an effective amount of:
the compound of any one of claims 1-71, or salt, solvate, hydrate, polymorph, co-crystal, tautomer, stereoisomer, isotopically labeled compound, or prodrug thereof; or
the pharmaceutical composition of any one of claims 72-75.

78. A method of preventing a disease in a subject in need thereof, the method comprising administering to the subject an effective amount of:
the compound of any one of claims 1-71, or salt, solvate, hydrate, polymorph, co-crystal, tautomer, stereoisomer, isotopically labeled compound, or prodrug thereof; or
the pharmaceutical composition of any one of claims 72-75.

79. The method of claim 77 or 78, wherein the effective amount is effective in inhibiting TEAD transcription activity.

80. The method of any one of claims 77-79, wherein the effective amount is about 1 mg/kg to about 200 mg/kg.

81. The method of any one of claims 77-80, wherein the effective amount is about 10 mg/kg to about 100 mg/kg.

82. The method of any one of claims 77-81, wherein the effective amount is about 20 mg/kg to about 55 mg/kg.

83. The method of any one of claims 77-82, wherein the effective amount is about 25 mg/kg.

84. The method of any one of claims 77-82, wherein the effective amount is about 50 mg/kg.

85. The method of any one of claims 77-84, wherein the disease is cancer.

86. The method of claim 85, wherein the cancer is breast, pancreatic, liver, colorectal, lung, ovarian, or prostate cancer.

87. The method of claim 85, wherein the cancer is hepatocellular carcinoma, triple negative breast cancer, mesothelioma, or hepatoblastoma.

88. The method of any one of claims 77-87, wherein the method further comprises co-administering an additional pharmaceutical agent.

89. The method of claim 88, wherein the additional pharmaceutical agent is co-administered on the same schedule as the compound, or salt, solvate, hydrate, polymorph, co-crystal, tautomer, stereoisomer, isotopically labeled compound, or prodrug thereof; or to the pharmaceutical composition.

90. The method of claim 88 or 89, wherein the co-administration increases sensitivity to the additional pharmaceutical agent.

91. The method of any one of claims 88-90, wherein the co-administration increases sensitivity to the compound, or salt, solvate, hydrate, polymorph, co-crystal, tautomer, stereoisomer, isotopically labeled compound, or prodrug thereof; or to the pharmaceutical composition.

92. The method of any one of claims 88-91, wherein the additional pharmaceutical agent is Sorafenib.

93. The method of any one of claims 88-92, wherein the additional pharmaceutical agent is administered in the amount of about 1 mg/kg to about 200 mg/kg.

94. The method of any one of claims 88-93, wherein the additional pharmaceutical agent is administered in the amount of about 50 mg/kg to about 150 mg/kg.

95. The method of any one of claims 88-94, wherein the additional pharmaceutical agent is administered in the amount of about 100 mg/kg.

96. A method of inhibiting the activity of TEAD in a subject in need thereof, the method comprising administering to the subject an effective amount of:
the compound of any one of claims 1-71, or salt, solvate, hydrate, polymorph, co-crystal, tautomer, stereoisomer, isotopically labeled compound, or prodrug thereof; or
the pharmaceutical composition of any one of claims 72-75.

97. The method of any one of claims 77-96, wherein the subject is a human.

98. The method of claim 97, wherein the subject is a human aged 18 and older.

99. A method of inhibiting the activity of TEAD in a cell, tissue, or biological sample the method comprising contacting the cell, tissue, or biological sample with an effective amount of:
the compound of any one of claims 1-71, or salt, solvate, hydrate, polymorph, co-crystal, tautomer, stereoisomer, isotopically labeled compound, or prodrug thereof; or
the pharmaceutical composition of any one of claims 72-75.

100. The method of claim 99, wherein the cell, tissue, or biological sample is in vitro.
